(19) United States
(12) Patent Application Publication
(10) Pub. No.: US 2013/0058948 A1
(43) Pub. Date: Mar. 7, 2013

(54) Antibodies Directed To Gpnmb And Uses Thereof

(71) Applicants: Michael Jeffers, Branford, CT (US); Ronit Simantov, New Haven, CT (US)

(72) Inventors: Michael Jeffers, Branford, CT (US); Ronit Simantov, New Haven, CT (US)

(21) Appl. No.: 13/445,730

(22) Filed: Apr. 12, 2012

Related U.S. Application Data
(63) Continuation of application No. 13/214,698, filed on Aug. 22, 2011, now abandoned.
Application No. 13/214,698 is a continuation of application No. 12/983,610, filed on Jan. 3, 2011, now abandoned.
Application No. 12/983,610 is a continuation of application No. 12/784,152, filed on May 20, 2010, now abandoned.
(60) Provisional application No. 61/179,813, filed on May 20, 2009.

(51) Int. Cl. A61K 39/395; A61P 35/04; A61P 35/00

(52) U.S. Cl. 424/156.1; 424/174.1; 424/178.1

(57) ABSTRACT

The present invention provides fully human monoclonal antibodies that specifically bind to GPNMB, and uses thereof. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDRs) are provided. The present invention also provides immunoconjugates comprising anti-GPNMB antibodies and methods of using such immunoconjugates. The present invention further provides methods of treating breast cancer using antibodies that bind to GPNMB, immunoconjugates and other derivatives thereof.

### RELATED APPLICATIONS

This application is a continuation of U.S. patent application Ser. No. 13/214,698, filed Aug. 22, 2011, which is a continuation of U.S. patent application Ser. No. 12/983,610, filed Jan. 3, 2011, which is a continuation of U.S. patent application Ser. No. 12/784,152, filed May 20, 2010, which claims the benefit of U.S. Provisional Application No. 61/179,813, filed May 20, 2009, the contents of which are hereby incorporated by reference in their entirety.

### INCORPORATION BY REFERENCE OF SEQUENCE LISTING

The contents of the text file named “CO9USSeqList.txt,” which was created on Aug. 22, 2011 and is 99.2 KB in size, are hereby incorporated by reference in their entirety.

### FIELD OF THE INVENTION

The present invention relates to antibodies with specificity to GPNMB, and uses of such antibodies. In particular, the present invention provides fully human monoclonal antibodies that specifically bind to GPNMB, immunoconjugates that include such antibodies, and uses thereof. The present invention further provides methods of treating breast cancer using antibodies that bind to GPNMB, immunoconjugates and other derivatives thereof.

### BACKGROUND OF THE INVENTION

A putative transmembrane glycoprotein called “nmb” (Acc. No. X76534 EMBL), referred to herein as GPNMB, was identified and described by Weterman et al., (Int J Cancer 60:73-81, 1995) as differentially expressed in low-metastatic human melanoma cancer cell lines and xenografts, compared to a more aggressive melanoma cell line. GPNMB shares 33% identity with the precursor of pMe117 melanocyte-specific protein (Kwon et al., 1991, PNAS 88:9228-9232). GPNMB is 71% homologous to a dendritic cell-associated transmembrane protein, DC-HIL (Shikano et al., 2001 Biol. Chem. 276:8125-8134). GPNMB is also known as the hematopoietic growth factor inducible neurokinin-1 protein HGFIN (Bandari et al, Reg. Peptides 111:169-178) and the bone-related gene osteoactivin (Owen et al. Crit Rev Eukaryot Gene Expr 2003, 13(2-4):205-220).

It would be desirable to have an antibody suitable for in vivo targeting of GPNMB and GPNMB-related pathologies, particularly cancers and more particularly, breast cancers and glioblastoma.

### SUMMARY OF THE INVENTION

The current invention provides human monoclonal antibodies that specifically bind GPNMB as well as variants, derivatives and antigen binding fragments of such antibodies. The invention further provides methods of treating breast cancer in a subject using antibodies that bind to GPNMB, immunoconjugates and other derivatives thereof. In some embodiments, the invention provides methods of treating triple negative or basal-like breast cancer in a subject using antibodies that bind to GPNMB, immunoconjugates and other derivatives thereof.

In some embodiments of the invention, the isolated antibody has a heavy chain variable region polypeptide comprising an amino acid sequence selected from the group consisting of SEQ ID NOs: 2, 20, 38, 56, 74, 92, 110, 128, 146, 164, 182, 200, 218, 236 and 254 or an amino acid sequence at least 85%, 90%, 91%, 92%, 93%, 94%, 95%, 96%, 97% 98%, 99% or more identical to a sequence selected from the group consisting of SEQ ID NOs: 2, 20, 38, 56, 74, 92, 110, 128, 146, 164, 182, 200, 218, 236 and 254. Such amino acid sequences can be encoded by nucleotide sequences selected from the group consisting of SEQ ID NOs: 1, 19, 37, 55, 73, 91, 109, 127, 145, 163, 181, 199, 217, 235 and 253 or an amino acid sequence at least 85%, 90%, 91%, 92%, 93%, 94%, 95%, 96%, 97% 98%, 99% or more identical to a sequence selected from the group consisting of SEQ ID NOs: 1, 19, 37, 55, 73, 91, 109, 127, 145, 163, 181, 199, 217, 235 and 253.

In some embodiments, the invention uses an isolated antibody that specifically binds to GPNMB and has a light chain variable region polypeptide comprising an amino acid sequence selected from the group consisting of SEQ ID NOs: 11, 29, 47, 65, 83, 101, 119, 137, 155, 173, 191, 209, 227, 245 and 263 or an amino acid sequence at least 85%, 90%, 91%, 92%, 93%, 94%, 95%, 96%, 97% 98%, 99% or more identical to a sequence selected from the group consisting of SEQ ID NOs: 11, 29, 47, 65, 83, 101, 119, 137, 155, 173, 191, 209, 227, 245 and 263. Such amino acid sequences can be encoded by nucleotide sequences selected from the group consisting of SEQ ID NOs: 10, 28, 46, 64, 82, 100, 118, 136, 154, 172, 190, 208, 226, 244 and 262 or an amino acid sequence at least 85%, 90%, 91%, 92%, 93%, 94%, 95%, 96%, 97% 98%, 99% or more identical to a sequence selected from the group consisting of SEQ ID NOs: 10, 28, 46, 64, 82, 100, 118, 136, 154, 172, 190, 208, 226, 244 and 262.

In some embodiments, the anti-GPNMB antibody has a heavy chain variable region polypeptide comprising an amino acid sequence selected from the group consisting of SEQ ID NOs: 2, 20, 38, 56, 74, 92, 110, 128, 146, 164, 182, 200, 218, 236 and 254 or an amino acid sequence at least 85%, 90%, 91%, 92%, 93%, 94%, 95%, 96%, 97% 98%, 99% or more identical to a sequence selected from the group consisting of SEQ ID NOs: 2, 20, 38, 56, 74, 92, 110, 128, 146, 164, 182, 200, 218, 236 and 254 and also includes a light chain variable region polypeptide comprising an amino acid sequence selected from the group consisting of SEQ ID NOs: 11, 29, 47, 65, 83, 101, 119, 137, 155, 173, 191, 209, 227, 245 and 263 or an amino acid sequence at least 85%, 90%, 91%, 92%, 93%, 94%, 95%, 96%, 97% 98%, 99% or more identical to a sequence selected from the group consisting of SEQ ID NOs: 11, 29, 47, 65, 83, 101, 119, 137, 155, 173, 191, 209, 227, 245 and 263.

The heavy chain CDRs include a VH CDR1 region comprising an amino acid sequence at least 85%, 90%, 91%, 92%, 93%, 94%, 95%, 96%, 97% 98%, 99% or more identical to a sequence selected from the group consisting of SEQ ID NOs: 4, 22, 40, 58, 76, 94, 112, 130, 148, 166, 184, 202, 220, 238 and 256; a VH CDR2 region comprising an amino acid sequence at least 85%, 90%, 91%, 92%, 93%, 94%, 95%, 96%, 97% 98%, 99% or more identical to a sequence selected from the group consisting of SEQ ID NOS: 5, 24, 42, 60, 78, 96, 114, 132, 150, 168, 186, 204, 222, 240 and 258; and a VH CDR3 region comprising an amino acid sequence at least 85%, 90%, 91%, 92%, 93%, 94%, 95%, 96%, 97% 98%, 99% or more identical to a sequence selected from the group consisting of SEQ ID NOS: 8, 26, 44, 62, 80, 98, 116, 134, 152, 170, 188, 206, 224, 242 and 260. The three light chain CDRs include a VL CDR1 region comprising an amino acid sequence at least 85%, 90%, 91%, 92%, 93%, 94%, 95%, 96%, 97% 98%, 99% or more identical to a sequence selected from the group consisting of SEQ ID NOS: 13, 31, 49, 67, 85, 103, 121, 139, 157, 175, 193, 211, 229, 247 and 265; a VL CDR2 region comprising an amino acid sequence at least 85%, 90%, 91%, 92%, 93%, 94%, 95%, 96%, 97% 98%, 99% or more identical to a sequence selected from the group consisting of SEQ ID NOS: 15, 33, 51, 69, 87, 105, 123, 141, 159, 177, 195, 213, 231, 249 and 267; and a VL CDR3 region comprising an amino acid sequence at least 85%, 90%, 91%, 92%, 93%, 94%, 95%, 96%, 97% 98%, 99% or more identical to a sequence selected from the group consisting of SEQ ID NOS: 17, 35, 53, 71, 89, 107, 135, 143, 161, 179, 197, 215, 233, 251 and 269.

Preferably, the heavy chain CDRs include a VH CDR1 region comprising an amino acid sequence at least 85%, 90%, 91%, 92%, 93%, 94%, 95%, 96%, 97% 98%, 99% or more identical to the amino acid sequence of SEQ ID NO: 22; a VH CDR2 region comprising an amino acid sequence at least 85%, 90%, 91%, 92%, 93%, 94%, 95%, 96%, 97% 98%, 99% or more identical to the amino acid sequence of SEQ ID NO: 24; and a VH CDR3 region comprising an amino acid sequence at least 85%, 90%, 91%, 92%, 93%, 94%, 95%, 96%, 97% 98%, 99% or more identical to the amino acid sequence of SEQ ID NO: 26. The three light chain CDRs include a VL CDR1 region comprising an amino acid sequence at least 85%, 90%, 91%, 92%, 93%, 94%, 95%, 96%, 97% 98%, 99% or more identical to the amino acid sequence of SEQ ID NO: 31; a VL CDR2 region comprising an amino acid sequence at least 85%, 90%, 91%, 92%, 93%, 94%, 95%, 96%, 97% 98%, 99% or more identical to the amino acid sequence of SEQ ID NO: 33; and a VL CDR3 region comprising an amino acid sequence at least 85%, 90%, 91%, 92%, 93%, 94%, 95%, 96%, 97% 98%, 99% or more identical to the amino acid sequence of SEQ ID NO: 35.

In particular embodiments, human anti-GPNMB antibodies include the Mab1.10.2, Mab1.15.1, Mab1.2.2, Mab1.7.1, Mab2.10.2, Mab2.15.1, Mab2.16.1, Mab2.17.1, Mab2.21.2, Mab2.22.1, Mab2.24.1, Mab2.3.1, Mab2.7.1, and Mab2.8.1 antibodies, which are described in WO2006/071441.

In one embodiment, the present invention provides an antibody, antibody-drug conjugate, or binding fragment thereof, that binds to GPNMB, wherein said antibody, antibody-drug conjugate or binding fragment thereof, neutralizes a GPNMB-induced activity, and wherein said antibody, or binding fragment thereof, cross-reacts with a fully human anti-GPNMB antibody described herein, preferably the CR011 antibody, the CDX-011 antibody-drug conjugate, also known as glembatumumab vedotin, or antigen-binding fragment thereof.

The anti-GPNMB antibodies, conjugates and other derivatives thereof may in certain embodiments exhibit a neutralizing ability and may inhibit one or more biological functions of GPNMB. The anti-GPNMB antibodies, conjugates and other derivatives thereof are able to bind GPNMB and may in certain embodiments modulate, e.g., inhibit, one or more biological functions of GPNMB.

In some embodiments, the anti-GPNMB antibody is an anti-GPNMB antibody described herein, an anti-GPNMB antibody-drug conjugate described herein, or an antibody or antibody-drug conjugate that binds to the same epitope as the anti-GPNMB antibody described herein or otherwise cross-competes with the binding site of the anti-GPNMB antibody described herein. In a preferred embodiment, the anti-GPNMB antibody is the anti-GPNMB antibody described herein and referred to as Mab1.15.1 or CR011, or the anti-GPNMB antibody-drug conjugate described herein and referred to as CDX-011 or glembatumumab vedotin, or an antibody or antibody-drug conjugate that binds to the same epitope as CR011 and glembatumumab vedotin described herein or otherwise cross-competes with the binding site of CR011 and glembatumumab vedotin.

The anti-GPNMB antibodies, conjugates and other derivatives thereof described herein are used in methods of treating, delaying the progression of, alleviating a symptom of, or otherwise ameliorating breast cancer in a subject. Overexpression of GPNMB promotes metastases to tissues and/or organs such as bone and lung. Overexpression of GPNMB promotes invasion of normal tissues by cancer cells and adhesion to endothelium. GPNMB mRNA is up-regulated in human breast cancer cell lines, and expression of GPNMB correlates with more aggressive phenotype(s) of cancers. See e.g., Rose et al., Mol. Cancer Res., vol. 5(1): 1001-1014 (2007) and Rose et al., “Glycoprotein nonmetastatic B is an independent prognostic indicator of recurrence and a novel therapeutic target in breast cancer,” Clin Cancer Res. 2010 Apr. 1; 16(7):2147-56, the contents of each of these references being hereby incorporated by reference in their entirety.

GPNMB is strongly over-expressed in about 25% of breast cancer cell lines. (See e.g., Neve et al., Cancer Cell, vol. 10: 515-527 (2006), the contents of which are hereby incorporated by reference in their entirety). High expression of GPNMB strongly correlated with ER negative tumors. HER2 was also over-expressed in about 25% of breast cancer cell lines. Minimal concurrent over-expression of GPNMB and HER2 was detected. In vitro data indicates that the anti-GPNMB antibodies described herein are useful in treating a variety of breast cancer patient populations.

In one embodiment, the anti-GPNMB antibodies, conjugates and other derivatives thereof are used to treat, delay the progression of, alleviate a symptom of, or otherwise ameliorate breast cancer in a patient who is non-responsive or no longer responsive to other breast cancer treatments, such as, for example, Trastuzumab (Herceptin®).

In one embodiment, the anti-GPNMB antibodies, conjugates and other derivatives thereof are used to treat, delay the progression of, alleviate a symptom of, or otherwise ameliorate estrogen receptor-negative, progesterone receptor-negative, human epidermal growth factor receptor 2 (HER2) negative or triple-negative breast cancer in a subject. Triple negative breast cancer includes tumor(s) that are estrogen receptor-negative, progesterone receptor-negative and human epidermal growth factor receptor 2 (HER2) negative.

In one embodiment, the anti-GPNMB antibodies, conjugates and other derivatives thereof are used to treat, delay the progression of, alleviate a symptom of, or otherwise ameliorate a locally advanced and/or metastatic breast cancer in a subject. “Locally advanced” breast cancer includes, for example, breast cancers that exhibit one or more of the following characteristics: a tumor greater than 5 cm across, a fixed lump in the axilla (i.e., underarm), ulceration of the skin, involvement of the deep chest muscles, involvement of multiple lymph nodes in the local area including, e.g., those located in the axilla and/or in the soft tissues above or below the collarbone. Locally advanced breast cancers typically involve the local area of the breast and the axilla and the lymph nodes contained therein. Such cancers are usually large, but localized to these areas. “Metastatic” breast cancer includes breast cancers where the disease has spread to other areas of the body, such as the lung, liver, brain, skin or bone, i.e., one or more metastases is found in an area of the body outside of the breast, including for example, the lung, liver, brain, skin or bone.

In some embodiments, the anti-GPNMB antibodies, conjugates and other derivatives thereof are used in conjunction with any of a variety of known treatments for locally advanced and/or metastatic including, by way of non-limiting example, surgical treatments and methods, radiation therapy, chemotherapy and/or hormone or other endocrine-related treatment.

The invention also provides methods of using the anti-GPNMB antibodies, conjugates and other derivatives thereof to enhance or supplement a breast cancer therapy in a subject, where the subject is receiving or has been received or otherwise been administered this breast cancer treatment in an amount or in a regimen, dosing or other administration schedule that is sufficient to produce a therapeutic outcome in the subject such as, for example, treating, delaying the progression of, alleviating a symptom of, or otherwise ameliorating a symptom of a breast cancer in the subject. In some embodiments, the subject is receiving or has previous received or otherwise been administered a breast cancer therapy such as surgical intervention, radiation therapy, chemotherapy and/or hormone or other endocrine-related treatment. In some embodiments of these methods and uses, the subject is non-responsive, less responsive or otherwise exhibits a decrease in responsiveness to the breast cancer therapy.

In some embodiments of these methods and uses, the anti-GPNMB antibody, conjugate and other derivative thereof is used in an amount that is sufficient to reduce the dosage of a breast cancer therapy such as, e.g., radiation therapy, chemotherapy and/or hormone or other endocrine-related treatment, that is needed to produce the desired therapeutic outcome in the subject. In some embodiments of these uses, the anti-GPNMB antibody, conjugate and other derivative thereof is used in an amount that is sufficient to decrease the frequency of administration of a breast cancer therapy such as, e.g., radiation therapy, chemotherapy and/or hormone or other endocrine-related treatment, that is needed to produce the desired therapeutic outcome in the subject.

In some embodiments, the breast cancer is a basal-like or triple negative breast cancer. In some embodiments, the breast cancer is a locally advanced and/or metastatic breast cancer.

The subject may be suffering from or is predisposed to developing a breast cancer, such as, for example, estrogen receptor-negative, progesterone receptor-negative, human epidermal growth factor receptor 2 (HER2) negative or triple negative breast cancer. Preferably, the subject is a mammal, and more preferably, the subject is a human.

The anti-GPNMB antibodies, antibody-drug conjugates and derivatives thereof are administered to a subject in need thereof. The anti-GPNMB antibodies, antibody-drug conjugates and derivatives thereof are administered in an amount effective to treat, delay the progression of, alleviate a symptom of, or otherwise ameliorate a breast cancer. In some embodiments, the effective amount is a unit dose between 0.1 mg/kg to 10 mg/kg, with 2 to 4 or more administrations. For example, the effective amount is a unit dose between 0.1 mg/kg to 2 mg/kg. In some embodiments, the effective amount is a unit dose about 1 mg/kg.

In some embodiments, the anti-GPNMB antibodies, antibody-drug conjugates and derivatives thereof are administered to a subject in an 18 to 25 day cycle. In some embodiments, the anti-GPNMB antibodies, antibody-drug conjugates and derivatives thereof are administered to a subject in a 21 day cycle. In some embodiments, the anti-GPNMB antibodies, antibody-drug conjugates and derivatives thereof are administered to a subject in a adjuvant setting. In some embodiments, the anti-GPNMB antibodies, antibody-drug conjugates and derivatives thereof are administered to a subject in a neoadjuvant setting. In some embodiments, the anti-GPNMB antibodies, antibody-drug conjugates and derivatives thereof are administered to a subject in a metastatic setting.

In one embodiment, the present invention provides naked IgG1 anti-GPNMB antibodies that have cytotoxic effect to cells overexpressing GPNMB. In a specific embodiment, the present invention provides methods of treating or preventing diseases associated with overexpression of GPNMB comprising administering to a subject in need thereof a composition comprising a naked IgG1 anti-GPNMB antibody and an immunomodulator (such as, but not limited to, interferons and cytokines).

The invention provides methods for treating, delaying the progression of alleviating a symptom of, or otherwise ameliorating breast cancer in a subject in need thereof, by administering an isolated antibody that specifically binds to GPNMB or a conjugate of such an antibody. The invention also provides isolated antibodies that specifically bind to GPNMB or a conjugate of such an antibody for use in treating, delaying the progression of, alleviating a symptom of, or otherwise ameliorating breast cancer in a subject in need thereof. The invention also provides uses of isolated antibodies that specifically binds to GPNMB or a conjugate of such an antibody in the manufacture of medicaments for treating, delaying the progression of, alleviating a symptom of, or otherwise ameliorating breast cancer in a subject in need thereof.

In some embodiments of these methods and uses, the breast cancer is an estrogen receptor-negative, progesterone receptor-negative or human epidermal growth factor receptor 2 (HER2) negative breast cancer.

In some embodiments of these methods and uses, the breast cancer is metastatic. In some embodiments of these methods and uses, the level of GPNMB expression or overexpression is first detected in a biological sample from the subject.

In some embodiments of these methods and uses, the antibody is monoclonal antibody. In some embodiments of these methods and uses, the antibody is a human antibody. In some embodiments of these methods and uses, the antibody is a fully human antibody.

In some embodiments of these methods and uses, the antibody specifically binds GPNMB with an affinity constant greater than 10⁷M⁻¹. In some embodiments of these methods and uses, the antibody includes a region selected from the group: VH1-2. VH2-5, VH3-11, VH3-21, VH3-30, VH3-33, VH4-31, VH4-59 and VH5-51 or a region derived from a region selected from the group: VH1-2, VH2-5, VH3-11, VH3-21, VH3-30, VH3-33, VH4-31, VH4-59 and VH5-51. In some embodiments of these methods and uses, the antibody includes a region selected from the group: A2, A3, A20, A27, A30, L2 and O1 or a region derived from a region selected from the group: A2, A3, A20, A27, A30, L2 and O1.

In some embodiments of these methods and uses, the antibody includes an amino acid sequence selected from the group comprising: SEQ ID NO: 2, 20, 38, 56, 74, 92, 110, 128, 146, 164, 182, 200, 218, 236 and 254. In some embodiments of these methods and uses, the antibody includes an amino acid sequence selected from the group: SEQ ID NO: 11, 29, 47, 65, 83, 101, 119, 137, 155, 173, 191, 209, 227, 245 and 263.

In some embodiments of these methods and uses, the antibody includes (a) a V_{H} CDR1 region comprising the amino acid sequence of SEQ ID NO: 22, 4, 40, 58, 76, 94, 112, 130, 148, 166, 184, 202, 220, 238 or 256; (b) a V_{H} CDR2 region comprising the amino acid sequence of SEQ ID NO: 24, 5, 42, 60, 78, 96, 114, 132, 150, 168, 186, 204, 222, 240 or 258; (c) a V_{H} CDR3 region comprising the amino acid sequence of SEQ ID NO: 26, 8, 44, 62, 80, 98, 116, 134, 152, 170, 188, 206, 224, 242 or 260, (d) a V_{L} CDR1 region comprising the amino acid sequence of SEQ ID NO: 31, 13, 49, 67, 85, 103, 121, 139, 157, 175, 193, 211, 229, 247 or 265; (e) a V_{L} CDR2 region comprising the amino acid sequence of SEQ ID NO: 33, 15, 51, 69, 87, 105, 123, 141, 159, 177, 195, 213, 231, 249 or 267; and (f) a V_{L} CDR3 region comprising the amino acid sequence of SEQ ID NO: 35, 17; 53, 71, 89, 107, 135, 143, 161, 179, 197, 215, 233, 251 or 269.

In some embodiments of these methods and uses, the antibody is selected from the group: Mab 1.10.2, Mab 1.15.1, Mab 1.2.2, Mab 1.7.1, Mab 2.10.2, Mab 2.15.1, Mab 2.16.1, Mab 2.17.1, Mab 2.21.2, Mab 2.22.1, Mab 2.24.1, Mab 2.3.1, Mab 2.7.1 and Mab 2.8.1, which are described in WO2006/071441.

In some embodiments of these methods and uses, the antibody is an IgG1 or IgG2 antibody. In some embodiments of these methods and uses, the antibody is in the form of an immunoconjugate that includes any of the antibodies described herein and a cytotoxic agent. In some embodiments of these methods and uses, the cytotoxic agent is auristatin E (dolastatin-10) or a derivative thereof. In some embodiments of these methods and uses, the antibody is Mab 1.15.1 as described in WO2006/071441. In some embodiments of these methods and uses, the immunoconjugate is glembatumumab vedotin.

In some embodiments of these methods and uses, the subject is human.

In some embodiments of these methods and uses, the effective amount of an antibody or immunoconjugate described herein is a unit dose between 0.1 mg/kg to 10 mg/kg, with 2 to 4 administrations. In some embodiments of these methods and uses, the effective amount is a unit dose between 0.1 mg/kg to 2 mg/kg. In some embodiments of these methods and uses, the effective amount is a unit dose about 1 mg/kg. In some embodiments of these methods and uses, the antibody or conjugate (i.e., immunoconjugate) is administered in an 18 to 25 day cycle. In some embodiments of these methods and uses, the antibody or conjugate is administered in a 21 day cycle. In some embodiments of these methods and uses, the treatment or use is in an adjuvant setting. In some embodiments of these methods and uses, the treatment or use is in a neoadjuvant setting. In some embodiments of these methods and uses, the treatment or use is in a metastatic setting.

In some embodiments of these methods and uses, the antibody or conjugate is administered parenterally and/or formulated for parenteral administration. In some embodiments of these methods and uses, the antibody or conjugate is administered intravenously and/or formulated for intravenous administration.

The invention provides methods for treating, delaying the progression of alleviating a symptom of or otherwise ameliorating breast cancer in a subject who is non-responsive or no longer responsive to a previous treatment, by administering an isolated antibody that specifically binds to GPNMB or a conjugate of such an antibody. The invention also provides isolated antibodies that specifically bind to GPNMB or a conjugate of such an antibody for use in treating, delaying the progression of alleviating a symptom of, or otherwise ameliorating breast cancer in a subject who is non-responsive or no longer responsive to a previous treatment. The invention also provides uses of isolated antibodies that specifically binds to GPNMB or a conjugate of such an antibody in the manufacture of medicaments for treating, delaying the progression of, alleviating a symptom of, or otherwise ameliorating breast cancer in a subject who is non-responsive or no longer responsive to a previous treatment.

In some embodiments of these methods and uses, the breast cancer is metastatic. In some embodiments of these methods and uses, the level of GPNMB expression or overexpression is first detected in a biological sample from the subject.

In some embodiments of these methods and uses, the antibody is monoclonal antibody. In some embodiments of these methods and uses, the antibody is a human antibody. In some embodiments of these methods and uses, the antibody is a fully human antibody.

In some embodiments of these methods and uses, the antibody specifically binds GPNMB with an affinity constant greater than 10⁷M⁻¹. In some embodiments of these methods and uses, the antibody includes a region selected from the group: VH1-2, VH2-5, VH3-11, VH3-21, VH3-30, VH3-33, VH4-31, VH4-59 and VH5-51 or a region derived from a region selected from the group: VH1-2, VH2-5, VH3-11, VH3-21, VH3-30, VH3-33, VH4-31, VH4-59 and VH5-51. In some embodiments of these methods and uses, the antibody includes a region selected from the group: A2, A3, A20, A27, A30, L2 and O1 or a region derived from a region selected from the group: A2, A3, A20, A27, A30, L2 and O1.

In some embodiments of these methods and uses, the antibody includes an amino acid sequence selected from the group comprising: SEQ ID NO: 2, 20, 38, 56, 74, 92, 110, 128, 146, 164, 182, 200, 218, 236 and 254. In some embodiments of these methods and uses, the antibody includes an amino acid sequence selected from the group: SEQ ID NO: 11, 29, 47, 65, 83, 101, 119, 137, 155, 173, 191, 209, 227, 245 and 263.

In some embodiments of these methods and uses, the antibody includes (a) a V_{H} CDR1 region comprising the amino acid sequence of SEQ ID NO: 22, 4, 40, 58, 76, 94, 112, 130, 148, 166, 184, 202, 220, 238 or 256; (b) a V_{H} CDR2 region comprising the amino acid sequence of SEQ ID NO: 24, 5, 42, 60, 78, 96, 114, 132, 150, 168, 186, 204, 222, 240 or 258; (c) a V_{H} CDR3 region comprising the amino acid sequence of SEQ ID NO: 26, 8, 44, 62, 80, 98, 116, 134, 152, 170, 188, 206, 224, 242 or 260, (d) a V_{L} CDR1 region comprising the amino acid sequence of SEQ ID NO: 31, 13, 49, 67, 85, 103, 121, 139, 157, 175, 193, 211, 229, 247 or 265; (e) a V_{L} CDR2 region comprising the amino acid sequence of SEQ ID NO: 33, 15, 51, 69, 87, 105, 123, 141, 159, 177, 195, 213, 231, 249 or 267; and (f) a V_{L} CDR3 region comprising the amino acid sequence of SEQ ID NO: 35, 17, 53, 71, 89, 107, 135, 143, 161, 179, 197, 215, 233, 251 or 269.

In some embodiments of these methods and uses, the antibody is selected from the group: Mab 1.10.2, Mab 1.15.1, Mab 1.2.2, Mab 1.7.1, Mab 2.10.2, Mab 2.15.1, Mab 2.16.1, Mab 2.17.1, Mab 2.21.2, Mab 2.22.1, Mab 2.24.1, Mab 2.3.1, Mab 2.7.1 and Mab 2.8.1, which are described in WO2006/071441.

In some embodiments of these methods and uses, the antibody is an IgG1 or IgG2 antibody. In some embodiments of these methods and uses, the antibody is in the form of an immunoconjugate that includes any of the antibodies described herein and a cytotoxic agent. In some embodiments of these methods and uses, the cytotoxic agent is auristatin E (dolastatin-10) or a derivative thereof. In some embodiments of these methods and uses, the antibody is Mab 1.15.1 as described in WO2006/071441. In some embodiments of these methods and uses, the immunoconjugate is glembatumumab vedotin.

In some embodiments of these methods and uses, the subject is human.

In some embodiments of these methods and uses, the effective amount of an antibody or immunoconjugate described herein is a unit dose between 0.1 mg/kg to 10 mg/kg, with 2 to 4 administrations. In some embodiments of these methods and uses, the effective amount is a unit dose between 0.1 mg/kg to 2 mg/kg. In some embodiments of these methods and uses, the effective amount is a unit dose about 1 mg/kg. In some embodiments of these methods and uses, the antibody or conjugate (i.e., immunoconjugate) is administered in an 18 to 25 day cycle. In some embodiments of these methods and uses, the antibody or conjugate is administered in a 21 day cycle. In some embodiments of these methods and uses, the treatment or use is in an adjuvant setting. In some embodiments of these methods and uses, the treatment or use is in a neoadjuvant setting. In some embodiments of these methods and uses, the treatment or use is in a metastatic setting.

In some embodiments of these methods and uses, the antibody or conjugate is administered parenterally and/or formulated for parenteral administration. In some embodiments of these methods and uses, the antibody or conjugate is administered intravenously and/or formulated for intravenous administration.

The invention provides methods for treating, delaying the progression of, alleviating a symptom of, or otherwise ameliorating basal-like or triple-negative (TN) breast cancer in a subject in need thereof, by administering an isolated antibody that specifically binds to GPNMB or a conjugate of such an antibody. The invention also provides isolated antibodies that specifically bind to GPNMB or a conjugate of such an antibody for use in treating, delaying the progression of, alleviating a symptom of, or otherwise ameliorating basal-like or triple-negative (TN) breast cancer in a subject in need thereof. The invention also provides uses of isolated antibodies that specifically binds to GPNMB or a conjugate of such an antibody in the manufacture of medicaments for treating, delaying the progression of, alleviating a symptom of, or otherwise ameliorating basal-like or triple-negative (TN) breast cancer in a subject in need thereof.

In some embodiments of these methods and uses, the breast cancer is metastatic. In some embodiments of these methods and uses, the level of GPNMB expression or overexpression is first detected in a biological sample from the subject.

In some embodiments of these methods and uses, the antibody is monoclonal antibody. In some embodiments of these methods and uses, the antibody is a human antibody. In some embodiments of these methods and uses, the antibody is a fully human antibody.

In some embodiments of these methods and uses, the antibody specifically binds GPNMB with an affinity constant greater than 10⁷M⁻¹. In some embodiments of these methods and uses, the antibody includes a region selected from the group: VH1-2, VH2-5, VH3-11, VH3-21, VH3-30, VH3-33, VH4-31, VH4-59 and VH5-51 or a region derived from a region selected from the group: VH1-2, VH2-5, VH3-11, VH3-21, VH3-30, VH3-33, VH4-31, VH4-59 and VH5-51. In some embodiments of these methods and uses, the antibody includes a region selected from the group: A2, A3, A20, A27, A30, L2 and O1 or a region derived from a region selected from the group: A2, A3, A20, A27, A30, L2 and O1.

In some embodiments of these methods and uses, the antibody includes an amino acid sequence selected from the group comprising: SEQ ID NO: 2, 20, 38, 56, 74, 92, 110, 128, 146, 164, 182, 200, 218, 236 and 254. In some embodiments of these methods and uses, the antibody includes an amino acid sequence selected from the group: SEQ ID NO: 11, 29, 47, 65, 83, 101, 119, 137, 155, 173, 191, 209, 227, 245 and 263.

In some embodiments of these methods and uses, the antibody includes (a) a V_{H} CDR1 region comprising the amino acid sequence of SEQ ID NO: 22, 4, 40, 58, 76, 94, 112, 130, 148, 166, 184, 202, 220, 238 or 256; (b) a V_{H} CDR2 region comprising the amino acid sequence of SEQ ID NO: 24, 5, 42, 60, 78, 96, 114, 132, 150, 168, 186, 204, 222, 240 or 258; (c) a V_{H} CDR3 region comprising the amino acid sequence of SEQ ID NO: 26, 8, 44, 62, 80, 98, 116, 134, 152, 170, 188, 206, 224, 242 or 260, (d) a V_{L} CDR1 region comprising the amino acid sequence of SEQ ID NO: 31, 13, 49, 67, 85, 103, 121, 139, 157, 175, 193, 211, 229, 247 or 265; (e) a V_{L} CDR2 region comprising the amino acid sequence of SEQ ID NO: 33, 15, 51, 69, 87, 105, 123, 141, 159, 177, 195, 213, 231, 249 or 267; and (f) a V_{L} CDR3 region comprising the amino acid sequence of SEQ ID NO: 35, 17, 53, 71, 89, 107, 135, 143, 161, 179, 197, 215, 233, 251 or 269.

In some embodiments of these methods and uses, the antibody is selected from the group: Mab 1.10.2, Mab 1.15.1, Mab 1.2.2, Mab 1.7.1, Mab 2.10.2, Mab 2.15.1, Mab 2.16.1, Mab 2.17.1, Mab 2.21.2, Mab 2.22.1, Mab 2.24.1, Mab 2.3.1, Mab 2.7.1 and Mab 2.8.1, which are described in WO2006/071441.

In some embodiments of these methods and uses, the antibody is an IgG1 or IgG2 antibody. In some embodiments of these methods and uses, the antibody is in the form of an immunoconjugate that includes any of the antibodies described herein and a cytotoxic agent. In some embodiments of these methods and uses, the cytotoxic agent is auristatin E (dolastatin-10) or a derivative thereof. In some embodiments of these methods and uses, the antibody is Mab 1.15.1 as described in WO2006/071441. In some embodiments of these methods and uses, the immunoconjugate is glembatumumab vedotin.

In some embodiments of these methods and uses, the subject is human.

In some embodiments of these methods and uses, the effective amount of an antibody or immunoconjugate described herein is a unit dose between 0.1 mg/kg to 10 mg/kg, with 2 to 4 administrations. In some embodiments of these methods and uses, the effective amount is a unit dose between 0.1 mg/kg to 2 mg/kg. In some embodiments of these methods and uses, the effective amount is a unit dose about 1 mg/kg. In some embodiments of these methods and uses, the antibody or conjugate (i.e., immunoconjugate) is administered in an 18 to 25 day cycle. In some embodiments of these methods and uses, the antibody or conjugate is administered in a 21 day cycle. In some embodiments of these methods and uses, the treatment or use is in an adjuvant setting. In some embodiments of these methods and uses, the treatment or use is in a neoadjuvant setting. In some embodiments of these methods and uses, the treatment or use is in a metastatic setting.

In some embodiments of these methods and uses, the antibody or conjugate is administered parenterally and/or formulated for parenteral administration. In some embodiments of these methods and uses, the antibody or conjugate is administered intravenously and/or formulated for intravenous administration.

The invention provides methods for treating, delaying the progression of alleviating a symptom of, or otherwise ameliorating glioblastoma in a subject in need thereof, by administering an isolated antibody that specifically binds to GPNMB or a conjugate of such an antibody. The invention also provides isolated antibodies that specifically bind to GPNMB or a conjugate of such an antibody for use in treating, delaying the progression of, alleviating a symptom of, or otherwise ameliorating glioblastoma in a subject in need thereof. The invention also provides uses of isolated antibodies that specifically binds to GPNMB or a conjugate of such an antibody in the manufacture of medicaments for treating, delaying the progression of, alleviating a symptom of, or otherwise ameliorating glioblastoma in a subject in need thereof.

In another embodiment, the present invention provides immunoconjugates that comprise an anti-GPNMB antibody or a fragment thereof, and a cytotoxic agent. In a specific embodiment, the cytotoxic agent is auristatin E (dolastatin-10) or a derivative thereof.

Compositions comprising human anti-GPNMB antibodies, including therapeutic compositions comprising same, and methods of use are provided. Particularly, therapeutic immunoconjugates comprising anti-GPNMB antibodies and a cytotoxic or cytostatic agent for treating GPNMB expressing cancers, preferably breast cancers, are provided. Dosage regimens are also provided.

Pharmaceutical compositions according to the invention can include an antibody of the invention and a carrier. These pharmaceutical compositions can be included in kits, such as, for example, diagnostic kits.

Additional aspects of the disclosure will be set forth in part in the description which follows, and in part will be obvious from the description, or may be learned by practicing the invention.

### BRIEF DESCRIPTION OF THE FIGURES

FIG. 1 is a graph depicting tumor shrinkage in patients receiving anti-GPNMB therapy. The maximum percent decrease in the sum of the longest diameters of target lesions is plotted individually for each of the 16 patients with pre- and post-baseline tumor measurements.

FIG. 2 is a series of representative images illustrating immunohistochemistry results for GPNMB expression in patient biopsy samples. Scale bar represents 100 μm.

FIG. 3 is a graph depicting progression-free survival in patients by GPNMB expression status (all doses).

FIG. 4 is a graph depicting maximum tumor shrinkage in the Phase II studies provided herein.

FIG. 5A is an illustration of the chemical structure of Maleimidocoaproyl-Valine-Citrullin-Monomethyl-Auristatin E (vcMMAE), and FIG. 5B is an illustration depicting the anti-GPNMB antibody-drug conjugate CDX-011.

### DETAILED DESCRIPTION OF THE INVENTION

The present invention provides methods of treating, ameliorating, delaying the onset and/or progression or otherwise reducing the severity of a breast cancer or one or more symptoms thereof using antibodies that specifically bind GPNMB.

Glycoprotein nonmetastatic B (GPNMB), also known as nmb, osteoactivin, dendritic cell-heparin integrin ligand, or hematopoietic growth factor inducible neurokinin-1 type, is a type I transmembrane protein (Ripoll V M, Irvine K M, Ravasi T, Sweet M J, Hume D A. GPNMB is induced in macrophages by IFN-γ and lipopolysaccharide and acts as a feedback regulator of proinflammatory responses. J Immunol 2007; 178:6557-66; Tse K F, Jeffers M, Pollack V A, et al. CR011, a fully human monoclonal antibody-auristatin E conjugate, for the treatment of melanoma. Clin Cancer Res 2006; 12:1373-82; and Abdelmagid S M, Barbe M F, Rico M C, et al. Osteoactivin, an anabolic factor that regulates osteoblast differentiation and function. Exp Cell Res 2008; 314:2334-51). The human and murine orthologues of this protein are referred to as GPNMB and osteoactivin, respectively. GPNMB is expressed at higher levels in several malignant human tissues relative to corresponding normal tissue (Tse K F et al., Clin Cancer Res 2006; 12:1373-82; Onaga M, Ido A, Hasuike S, et al. Osteoactivin expressed during cirrhosis development in rats fed a choline-deficient, L-amino acid-defined diet, accelerates motility of hepatoma cells. J Hepatol 2003; 39:779-85; and Rich J N, Shi Q, Hjelmeland M, et al. Bone-related genes expressed in advanced malignancies induce invasion and metastasis in a genetically defined human cancer model. J Biol Chem 2003; 278: 15951-7). Moreover, ectopic overexpression of GPNMB/osteoactivin promotes the invasion and metastasis of hepatocellular carcinoma, glioma, and breast cancer cells (Onaga et al., J Hepatol 2003; 39:779-85; Rich et al., J Biol Chem 2003; 278:15951-7; and Rose A A, Pepin F, Russo C, Abou Khalil J E, Hallett M, Siegel P M. Osteoactivin promotes breast cancer metastasis to bone. Mol Cancer Res 2007; 5:1001-14). Given its role as a mediator of metastasis and its cell surface expression, GPNMB is an attractive candidate for cancer therapy. Thus, the invention provides antibodies that specifically bind GPNMB, immunoconjugates thereof, and methods of use these antibodies and/or immunoconjugates in treating cancer, preferably breast cancer in a subject, preferably a human subject.

Breast cancer is a heterogeneous disease with respect to its histopathology and response to treatment. Gene expression analyses have classified primary human breast tumors into distinct molecular subtypes, which include normal-like, luminal, human epidermal growth factor receptor 2-positive (HER2+), and basal-like breast cancers (Perou C M, Sorlie T, Eisen M B, et al. Molecular portraits of human breast tumours. Nature 2000; 406:747-52; and Sorlie T, Perou C M, Tibshirani R, et al. Gene expression patterns of breast carcinomas distinguish tumor subclasses with clinical implications. Proc Natl Acad Sci USA 2001; 98:10869-74), which has implications for disease management (Sorlie T, Tibshirani R, Parker J, et al. Repeated observation of breast tumor subtypes in independent gene expression data sets. Proc Natl Acad Sci USA 2003; 100:8418-23). Recent work indicates that tumors within a particular subtype display distinct organ-specific patterns of recurrence (Smid M, Wang Y, Zhang Y, et al. Subtypes of breast cancer show preferential site of relapse. Cancer Res 2008; 68:3108-14 and Dent R, Hanna W M, Trudeau M, Rawlinson E, Sun P, Narod S A. Pattern of metastatic spread in triple-negative breast cancer. Breast Cancer Res Treat 2009; 115:423-8). Basal-like breast cancers are more aggressive in nature, preferentially metastasize to the brain and lung, and are responsible for a disproportionate number of deaths (Fadare O, Tavassoli F A. Clinical and pathologic aspects of basal-like breast cancers. Nat Clin Pract Oncol 2008; 5:149-59). Luminal breast tumors are generally responsive to hormonal therapies (Moulder S, Hortobagyi G N. Advances in the treatment of breast cancer. Clin Pharmacol Ther 2008; 83:26-36), whereas HER2+ tumors are treated primarily with HER2-targeted therapies such as trastuzumab or lapatinib. In contrast, no targeted therapeutic is currently available for patients with triple-negative breast cancers. This deficiency in targeted treatment options, coupled with the frequency and pattern of metastasis associated with this subtype, accounts for the poor outcomes of patients with basal-like breast cancer.

Triple-negative tumors constitute an aggressive subtype of breast cancer that is associated with poor disease outcome. Currently, no targeted therapies are available that effectively treat triple-negative tumors. However, there is substantial molecular heterogeneity within this subtype and some patients with triple-negative tumors do not recur.

The present invention provides methods of treating, ameliorating, delaying the onset and/or progression or otherwise reducing the severity of a breast cancer or one or more symptoms thereof using antibodies, preferably monoclonal antibodies or antigen-binding fragments thereof that specifically bind GPNMB or a biologically active portion thereof. The antibody is e.g., a fully human monoclonal antibody, or an antigen-binding fragment thereof. For example, antibodies that are useful in the compositions and methods provided herein include the antibodies that bind GPNMB as described in PCT Publication WO2006/071441.

In certain embodiments, the anti-GPNMB antibodies may modulate, block, inhibit, reduce, antagonize, neutralize or otherwise interfere with the biological activity of GPNMB. For example, the anti-GPNMB antibodies may completely or partially inhibit GPNMB biological activity by partially or completely modulating, blocking, inhibiting, reducing antagonizing, neutralizing, or otherwise interfering with the binding of GPNMB to a binding partner, or otherwise partially or completely modulating, blocking, inhibiting, reducing, antagonizing, neutralizing GPNMB mediated activity.

The anti-GPNMB antibodies may be considered to completely modulate, block, inhibit, reduce, antagonize, neutralize or otherwise interfere with GPNMB biological activity when the level of GPNMB activity in the presence of the anti-GPNMB antibody is decreased by at least 95%, e.g., by 96%, 97%, 98%, 99% or 100% as compared to the level of GPNMB activity in the absence of binding with an anti-GPNMB antibody described herein. The anti-GPNMB antibodies may be considered to partially modulate, block, inhibit, reduce, antagonize, neutralize or otherwise interfere with GPNMB activity when the level of GPNMB activity in the presence of the anti-GPNMB antibody is decreased by less than 95%, e.g., 10%, 20%, 25%, 30%, 40%, 50%, 60%, 75%, 80%, 85% or 90% as compared to the level of GPNMB activity in the absence of binding with an anti-GPNMB antibody described herein.

In an embodiment of the invention, the isolated antibody has a heavy chain variable region polypeptide comprising an amino acid sequence selected from the group consisting of SEQ ID NOs: 2, 20, 38, 56, 74, 92, 110, 128, 146, 164, 182, 200, 218, 236 and 254 or an amino acid sequence at least 85%, 90%, 91%, 92%, 93%, 94%, 95%, 96%, 97% 98%, 99% or more identical to a sequence selected from the group consisting of SEQ ID NOs: 22, 20, 38, 56, 74, 92, 110, 128, 146, 164, 182, 200, 218, 236 and 254. Such amino acid sequences can be encoded by nucleotide sequences selected from the group consisting of SEQ ID NOs: 1, 19, 37, 55, 73, 91, 109, 127, 145, 163, 181, 199, 217, 235 and 253 or an amino acid sequence at least 85%, 90%, 91%, 92%, 93%, 94%, 95%, 96%, 97% 98%, 99% or more identical to a sequence selected from the group consisting of SEQ ID NOs: 1, 19, 37, 55, 73, 91, 109, 127, 145, 163, 181, 199, 217, 235 and 253. In another embodiment, the invention provides an isolated antibody that specifically binds to GPNMB and has a light chain variable region polypeptide comprising an amino acid sequence selected from the group consisting of SEQ ID NOs: 11, 29, 47, 65, 83, 101, 119, 137, 155, 173, 191, 209, 227, 245 and 263 or an amino acid sequence at least 85%, 90%, 91%, 92%, 93%, 94%, 95%, 96%, 97% 98%, 99% or more identical to a sequence selected from the group consisting of SEQ ID NOs: 11, 29, 47, 65, 83, 101, 119, 137, 155, 173, 191, 209, 227, 245 and 263. Such amino acid sequences can be encoded by nucleotide sequences selected from the group consisting of SEQ ID NOs: 10, 28, 46, 64, 82, 100, 118, 136, 154, 172, 190, 208, 226, 244 and 262 or an amino acid sequence at least 85%, 90%, 91%, 92%, 93%, 94%, 95%, 96%, 97% 98%, 99% or more identical to a sequence selected from the group consisting of SEQ ID NOs: 10, 28, 46, 64, 82, 100, 118, 136, 154, 172, 190, 208, 226, 244 and 262.

The heavy chain CDRs include a VH CDR1 region comprising an amino acid sequence at least 85%, 90%, 91%, 92%, 93%, 94%, 95%, 96%, 97% 98%, 99% or more identical to a sequence selected from the group consisting of SEQ ID NOS: 4, 22, 40, 58, 76, 94, 112, 130, 148, 166, 184, 202, 220, 238 and 256; a VH CDR2 region comprising an amino acid sequence at least 85%, 90%, 91%, 92%, 93%, 94%, 95%, 96%, 97% 98%, 99% or more identical to a sequence selected from the group consisting of SEQ ID NOS: 5, 24, 42, 60, 78, 96, 114, 132, 150, 168, 186, 204, 222, 240 and 258; and a VH CDR3 region comprising an amino acid sequence at least 85%, 90%, 91%, 92%, 93%, 94%, 95%, 96%, 97% 98%, 99% or more identical to a sequence selected from the group consisting of SEQ ID NOS: 8, 26, 44, 62, 80, 98, 116, 134, 152, 170, 188, 206, 224, 242 and 260. The three light chain CDRs include a VL CDR1 region comprising an amino acid sequence at least 85%, 90%, 91%, 92%, 93%, 94%, 95%, 96%, 97% 98%, 99% or more identical to a sequence selected from the group consisting of SEQ ID NOS: 13, 31, 49, 67, 85, 103, 121, 139, 157, 175, 193, 211, 229, 247 and 265; a VL CDR2 region comprising an amino acid sequence at least 85%, 90%, 91%, 92%, 93%, 94%, 95%, 96%, 97% 98%, 99% or more identical to a sequence selected from the group consisting of SEQ ID NOS: 15, 33, 51, 69, 87, 105, 123, 141, 159, 177, 195, 213, 231, 249 and 267; and a VL CDR3 region comprising an amino acid sequence at least 85%, 90%, 91%, 92%, 93%, 94%, 95%, 96%, 97% 98%, 99% or more identical to a sequence selected from the group consisting of SEQ ID NOS: 17, 35, 53, 71, 89, 107, 135, 143, 161, 179, 197, 215, 233, 251 and 269.

Preferably, the heavy chain CDRs include a VH CDR1 region comprising an amino acid sequence at least 85%, 90%, 91%, 92%, 93%, 94%, 95%, 96%, 97% 98%, 99% or more identical to the amino acid sequence of SEQ ID NO: 22; a VH CDR2 region comprising an amino acid sequence at least 85%, 90%, 91%, 92%, 93%, 94%, 95%, 96%, 97% 98%, 99% or more identical to the amino acid sequence of SEQ ID NO: 24; and a VH CDR3 region comprising an amino acid sequence at least 85%, 90%, 91%, 92%, 93%, 94%, 95%, 96%, 97% 98%, 99% or more identical to the amino acid sequence of SEQ ID NO: 26. The three light chain CDRs include a VL CDR1 region comprising an amino acid sequence at least 85%, 90%, 91%, 92%, 93%, 94%, 95%, 96%, 97% 98%, 99% or more identical to the amino acid sequence of SEQ ID NO: 31; a VL CDR2 region comprising an amino acid sequence at least 85%, 90%, 91%, 92%, 93%, 94%, 95%, 96%, 97% 98%, 99% or more identical to the amino acid sequence of SEQ ID NO: 33; and a VL CDR3 region comprising an amino acid sequence at least 85%, 90%, 91%, 92%, 93%, 94%, 95%, 96%, 97% 98%, 99% or more identical to the amino acid sequence of SEQ ID NO: 35.

In particular embodiments, human anti-GPNMB antibodies include the Mab1.10.2, Mab1.15.1, Mab1.2.2, Mab1.7.1, Mab2.10.2, Mab2.15.1, Mab2.16.1, Mab2.17.1, Mab2.21.2, Mab2.22.1, Mab2.24.1, Mab2.3.1, Mab2.7.1, and Mab2.8.1 antibodies, which are described in WO2006/071441. The amino acid and nucleic acid sequences for these antibodies are shown below.

Antibody 1.10.2 Heavy Chain Variable Region

(SEQ ID NO: 1)
5′ AGGTGCAGCTGCAGGAGTCGGGCCCAGGACTGGTGAAGCCTTCGGAGACCCTGTCCCTCACC
TGCACTGTCTCTGGTGACTCCATCAGTAATTACTACTGGAGCTGGATCCGGCAGCCCCCAGGGAA
GGGACTGGAGTGGATTGGGTATTTCTATTACAGTGGGAGCACCAACTACAACCCCTCCCTCAAGA
GTCGAGTCACCATATCAGTAGACACGTCCAAGAACCAGTTCTCCCTGAAACTGAGCTCTGTGACC
GCTGCGGACACGGCCGTGTATTACTGTGCGAGAGATAGGGGCTGGGCTGACTACTGGGGCCAGGG
AACCCTGGTCACCGTCTCCTCAGCC 3′
(SEQ ID NO: 2)
5′ QVQLQESGPGLVKPSETLSLTCTVSGDSISNYYWSWIRQPPGKGLEWIGYFYYSGSTNYNPS
LKSRVTISVDTSKNQFSLKLSSVTAADTAVYYCARDRGWADYWGQGTLVTVSSA 3′

TABLE 1.1.10.2
Heavy chain variable (V) region domains.

| REGION | SEQUENCE | AA RESIDUES* | SEQ ID NO: |
|---|---|---|---|
| FRI | QVQLQESGPGLVKPSETLSLTCTVS | 1-25 | 3 |
| CDR1 | GDSISNYYWS | 26-35 | 4 |
| FR2 | WIRQPPGKGLEWIG | 36-49 | 5 |
| CDR2 | YFYYSGSTNYNPSLKS | 50-65 | 6 |
| FR3 | RVTISVDTSKNQFSLKLSSVTAADTAVYYCAR | 66-97 | 7 |
| CDR3 | DRGWADY | 98-104 | 8 |
| FR4 | WGQGTLVTVSSA | 105-116 | 9 |

*AA Residues of SEQ ID NO: 2

Antibody 1.10.2 Light Chain Variable Region

(SEQ ID NO: 10)
5′ GAAATTGTGTTGACGCAGTCTCCAGGCACCCTGTCTTTGTCTCCA
GGGGAAAGGGCCACCCTCTCCTGCAGAACCAGTCAGAGTATTAGCAGC
AGCTATTTAGCCTGGTACCAGCAGAAACCTGGCCAGGTTCCCAGGCTC
CTCATCTATGGTGCTTCCAGCAGGGCCACTGGCATCCCAGACAGGTTC
AGTGGCAGTGGGTCTGGGACAGACTTCACTCTCACCATCAGCAGACTG
GAGCCTGAAGATTTTGCAGTGTATTATTGTCAGCAGTATGGTAGCTCG
ATCACCTTCGGCCAAGGGACACGACTGGAGATTAAACGA 3′
(SEQ ID NO: 11)
5′ EIVLTQSPGTLSLSPGERATLSCRTSQSISSSYLAWYQQKPGQVP
RLLIYGASSRATGIPDRFSGSGSGTDFTLTISRLEPEDFAVYYCQQYG
SSITFGQGTRLEIKR 3′

TABLE 2
1.10.2 Light chain variable (V) region domains.

| | | AA | SEQ |
|---|---|---|---|
| REGION | SEQUENCE | RESIDUES* | ID NO: |
| FRI | EIVLTQSPGTLSLSPGERATLSC | 1-23 | 12 |
| CDR1 | RTSQSISSSYLA | 24-35 | 13 |
| FR2 | WYQQKPGQVPRLLIY | 36-50 | 14 |
| CDR2 | GASSRAT | 51-57 | 15 |
| FR3 | GIPDRFSGSGSGTDFTLTISRLEPE | 58-89 | 16 |
| | DFAVYYC | | |
| CDR3 | QQYGSSIT | 90-97 | 17 |
| FR4 | FGQGTRLEIKR | 98-108 | 18 |

*AA Residues of SEQ ID NO: 11

Antibody 1.15.1 (CR011 Antibody) Heavy Chain Variable Region

(SEQ ID NO: 19)
5′ CAGGTGCAGCTGCAGGAGTCGGGCCCAGGACTGGTGAAGCCTTCA
CAGACCCTGTCCCTCACCTGCACTGTCTCTGGTGGCTCCATCAGCAGT
TTTAATTACTACTGGAGCTGGATCCGCCACCACCCAGGGAAGGGCCTG
GAGTGGATTGGGTACATCTATTACAGTGGGAGCACCTACTCCAACCCG
TCCCTCAAGAGTCGAGTTACCATATCAGTAGACACGTCTAAGAACCAG
TTCTCCCTGACGCTGAGCTCTGTGACTGCCGCGGACACGGCCGTGTAT
TACTGTGCGAGAGGGTATAACTGGAACTACTTTGACTACTGGGGCCAG
GGAACCCTGGTCACCGTCTCCTCAGCC 3′
(SEQ ID NO: 20)
5′ QVQLQESGPGLVKPSQTLSLTCTVSGGSISSFNYYWSWIRHHPGK
GLEWIGYIYYSGSTYSNPSLKSRVTISVDTSKNQFSLTLSSVTAADTA
VYYCARGYNWNYFDYWGQGTLVTVSSA 3′

TABLE 3
1.15.1 Heavy chain V region domains.

| | | AA | SEQ |
|---|---|---|---|
| REGION | SEQUENCE | RESIDUES* | ID NO: |
| FRI | QVQLQESGPGLVKPSQTLSLTCTVSG | 1-30 | 21 |
| | GSIS | | |
| CDR1 | SFNYYWS | 31-37 | 22 |
| FR2 | WIRHHPGKGLEWIG | 38-51 | 23 |
| CDR2 | YIYYSGSTYSNPSLKS | 52-67 | 24 |
| FR3 | RVTISVDTSKNQFSLTLSSVTAADTA | 68-99 | 25 |
| | VYYCAR | | |
| CDR3 | GYNWNYFDY | 100-108 | 26 |
| FR4 | WGQGTLVTVSSA | 109-120 | 27 |

*AA Residues of SEQ ID NO: 20

Antibody 1.15.1 (CR011 Antibody) Mature Heavy Chain (IgG2)

(SEQ ID NO: 272)
QVQLQESGPGLVKPSQTLSLTCTVSGGSISSFNYYWSWIRHHPGKGLE
WIGYIYYSGSTYSNPSLKSRVTISVDTSKNQFSLTLSSVTAADTAVYY
CARGYNWNYFDYWGQGTLVTVSSASTKGPSVFPLAPCSRSTSESTAAL
GCLVKDYFPEPVTVSWNSGALTSGVHTFPAVLQSSGLYSLSSVVTVPS
SNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTK
PREEQFNSTFRVVSVLTVVHQDWLNGKEYKCKVSNKGLPAPIEKTISK
TKGQPREPQVYTLPPSREEMTKNQVSLTCLVKGFYPSDIAVEWESNGQ
PENNYKTTPPMLDSDGSFFLYSKLTVDKSRWQQGNVFSCSVMHEALHN
HYTQKSLSLSPGK

Antibody 1.15.1 (CR011 Antibody) Light Chain Variable Region

(SEQ ID NO: 28)
5′ GAAATAGTGATGACGCAGTCTCCAGCCACCCTGTCTGTGTCTCCA
GGGGAAAGAGCCACCCTCTCCTGCAGGGCCAGTCAGAGTGTTGACAAC
AACTTAGTCTGGTACCAGCAGAAACCTGGCCAGGCTCCCAGGCTCCTC
ATCTATGGTGCATCCACCAGGGCCACTGGTATCCCAGCCAGGTTCAGT
GGCAGTGGGTCTGGGACAGAGTTCACTCTCACCATCAGTAGTCTGCAG
TCTGAAGATTTTGCAGTTTATTACTGTCAGCAGTATAATAACTGGCCT
CCGTGGACGTTCGGCCAAGGGACCAAGGTGGAAATCAAACGA 3′
(SEQ ID NO: 29)
5′ EIVMTQSPATLSVSPGERATLSCRASQSVDNNLVWYQQKPGQAPR
LLIYGASTRATGIPARFSGSGSGTEFTLTISSLQSEDFAVYYCQQYNN
WPPWTFGQGTKVEIKR 3′

TABLE 4
1.15.1 Light chain V region domains.

| | | AA | SEQ |
|---|---|---|---|
| REGION | SEQUENCE | RESIDUES* | ID NO: |
| FRI | EIVMTQSPATLSVSPGERATLSC | 1-23 | 30 |
| CDR1 | RASQSVDNNLV | 24-34 | 31 |
| FR2 | WYQQKPGQAPRLLIY | 35-49 | 32 |
| CDR2 | GASTRAT | 50-56 | 33 |
| FR3 | GIPARFSGSGSGTEFTLTISSLQSE | 57-88 | 34 |
| | DFAVYYC | | |
| CDR3 | QQYNNWPPWT | 89-98 | 35 |
| FR4 | FGQGTKVEIKR | 99-109 | 36 |

*AA Residues of SEQ ID NO: 29

Antibody 1.2.2 Heavy Chain Variable Region

(SEQ ID NO: 37)
5′ ATCACCTTGAAGGAGTCTGGTCCTACGCTGGTGAAACCCACACAG
ACCCTCACGCTGACCTGCACCTTCTCTGGGTTCTCACTCAGCGCTGGT
GGAGTGGGTGTGGGCTGGATCCGTCAGCCCCCAGGAAAGGCCCTGGAG
TGGCTTGCACTCATTTATTGGAATGATGATAAGCGCTACAGCCCATCT
CTGAGGAGCAGGCTCACCATCACCAAGGACACCTCCAAAAACCAGGTG
GTCCTTACAATTACCAACATGGACCCTGTGGACACAGCCACATATTAT
TGTGCACACAGTCACTATGATTACGATTGGGGGAGTTACTTTGACTAC
TGGGGCCAGGGAACCCTGGTCACCGTCTCCTCAGCC 3′
(SEQ ID NO: 38)
5′ ITLKESGPTLVKPTQTLTLTCTFSGFSLSAGGVGVGWIRQPPGKA
LEWLALIYWNDDKRYSPSLRSRLTITKDTSKNQVVLTITNMDPVDTAT
YYCAHSHYDYDWGSYFDYWGQGTLVTVSSA 3′

TABLE 5
1.2.2 Heavy chain V region domains.

| | | | SEQ |
|---|---|---|---|
| | | AA | ID |
| REGION | SEQUENCE | RESIDUES* | NO: |
| FRI | ITLKESGPTLVKPTQTLTLTCTFS | 1-24 | 39 |
| CDR1 | GFSLSAGGVGVG | 25-36 | 40 |
| FR2 | WIRQPPGKALEWLA | 37-50 | 41 |
| CDR2 | LIYWNDDKRYSPSLRS | 51-66 | 42 |
| FR3 | RLTITKDTSKNQVVLTITNMDPVD | 67-98 | 43 |
| | TATYYCAH | | |
| CDR3 | SHYDYDWGSYFDY | 99-111 | 44 |
| FR4 | WGQGTLVTVSSA | 112-123 | 45 |

*AA Residues of SEQ ID NO: 38

Antibody 1.2.2 Light Chain Variable Region

(SEQ ID NO: 46)
5′ GATATTGTGATGACCCAGACTCCACTCTCCCTGCCCGTCACCCCT
GGAGAGCCGGCCTCCATCTCCTGCAGGTCTAGTCAGAGCCTCTTGGAT
AGTGATGATGGAAACACCTATTTGGACTGGTACCTGCAGAAGCCAGGA
CAGTCTCCACAGCTCCTGATCTATACGCTTTCCTATCGGGCCTCTGGA
GTCCCAGACAGGTTCAGTGGCAGTGGGTCAGGCACTGATTTCACACTG
AACATCAGCAGGGTGGAGGCTGAGGATGTTGGAGTTTATTACTGCATG
CAACGTATAGAGTTTCCTATCACCTTCGGCCAAGGGACACGACTGGAG
ATTAAACGA 3′
(SEQ ID NO: 47)
5′ DIVMTQTPLSLPVTPGEPASISCRSSQSLLDSDDGNTYLDWYLQK
PGQSPQLLIYTLSYRASGVPDRFSGSGSGTDFTLNISRVEAEDVGVYY
CMQRIEFPITFGQGTRLEIKR 3′

TABLE 6
1.2.2 Light chain V region domains.

| | | | SEQ |
|---|---|---|---|
| | | AA | ID |
| REGION | SEQUENCE | RESIDUES* | NO: |
| FRI | DIVMTQTPLSLPVTPGEPASISC | 1-23 | 48 |
| CDR1 | RSSQSLLDSDDGNTYLD | 24-40 | 49 |
| FR2 | WYLQKPGQSPQLLIY | 41-55 | 50 |
| CDR2 | TLSYRAS | 56-62 | 51 |
| FR3 | GVPDRFSGSGSGTDFTLNISRVEAED | 63-94 | 52 |
| | VGVYYC | | |
| CDR3 | MQRIEFPIT | 95-103 | 53 |
| FR4 | FGQGTRLEIKR | 104-114 | 54 |

*AA Residues of SEQ ID NO: 47

Antibody 1.7.1 Heavy Chain Variable Region

(SEQ ID NO: 55)
5′ CAGGTGCAGCTGCAGGAGTCGGGCCCAGGACTGGTGAAGCCTTCA
CAGACCCTGTCCCTCACCTGCACTGTCTCTGGTGGCTCCATCAGCAGT
GCTAATTACTACTGGACCTGGATCCGCCAGCACCCAGGGAAGGGCCTG
GAGTGGATTGGGTACATCTATTACAGTGGGAGCACCTACTGCAACCCG
TCCCTCAAGAGTCGAGTTATCATATCAGTAGACACGTCTAAGAACCAG
TTCTCCCTGAAGCTGAGCTCTGTGACTGCCGCGGACACGGCCGTGTAT
TACTGTGCGAGAGGGTATAACTGGAACTACTTTGACTACTGGGGCCAG
GGAACCCTGGTCACCGTCTCCTCAGCC 3′
(SEQ ID NO: 56)
5′ QVQLQESGPGLVKPSQTLSLTCTVSGGSISSANYYWTWIRQHPGK
GLEWIGYIYYSGSTYCNPSLKSRVIISVDTSKNQFSLKLSSVTAADTA
VYYCARGYNWNYFDYWGQGTLVTVSSA 3′

TABLE 7
1.7.1 Heavy chain V region domains.

| | | | SEQ |
|---|---|---|---|
| | | AA | ID |
| REGION | SEQUENCE | RESIDUES* | NO: |
| FRI | QVQLQESGPGLVKPSQTLSLTCTV | 1-25 | 57 |
| CDR1 | GGSISSANYYWT | 26-37 | 58 |
| FR2 | WIRQHPGKGLEWIG | 38-51 | 59 |
| CDR2 | YIYYSGSTYCNPSLKS | 52-67 | 60 |
| FR3 | RVIISVDTSKNQFSLKLSSVTAAD | 68-99 | 61 |
| | TAVYYCAR | | |
| CDR3 | GYNWNYFDY | 100-108 | 62 |
| FR4 | WGQGTLVTVSSA | 109-120 | 63 |

*AA Residues of SEQ ID NO: 56

Antibody 1.7.1 Light Chain Variable Region

(SEQ ID NO: 64)
5′ GATATAGTGATGACGCAGTCTCCAGCCACCCTGTCTGTGTCTCCA
GGGGAAAGAGCCACCCTCTCCTGCAGGGCCAGTCAGAGTGTTAGCAGC
AACTTAGCCTGGTACCAGGAGAGACCTGGCCAGGCTCCCAGACTCCTC
ATCTATGGTGCATCCACCAGGGCCACTGGTATCCCAGCCAGGTTCAGT
GGCAGTGGGTCTGGGACAGAGTTCACTCTCACCATCAGCAGCCTGCAG
TCTGAAGATTTTGCAGTTTATTACTGTCAGCAGTATAATAAGTGGCCT
CCGTGGACGTTCGGCCAAGGGACCAAGGTGGAAATCGAACGAACT 3′
(SEQ ID NO: 65)
5′ DIVMTQSPATLSVSPGERATLSCRASQSVSSNLAWYQERPGQAPR
LLIYGASTRATGIPARFSGSGSGTEFTLTISSLQSEDFAVYYCQQYNK
WPPWTFGQGTKVEIER 3′

TABLE 8
1.7.1 Light chain V region domains.

| | | | SEQ |
|---|---|---|---|
| | | AA | ID |
| REGION | SEQUENCE | RESIDUES* | NO: |
| FRI | DIVMTQSPATLSVSPGERATLSC | 1-23 | 66 |
| CDR1 | RASQSVSSNLA | 24-34 | 67 |
| FR2 | WYQERPGQAPRLLIY | 35-49 | 68 |
| CDR2 | GASTRAT | 50-56 | 69 |
| FR3 | GIPARFSGSGSGTEFTLTISSLQSED | 57-88 | 70 |
| | FAVYYC | | |
| CDR3 | QQYNKWPPWT | 89-98 | 71 |
| FR4 | FGQGTKVEIER | 99-109 | 72 |

*AA Residues of SEQ ID NO: 65

Antibody 2.10.2 Heavy Chain Variable Region

(SEQ ID NO: 73)
5′ CAGCTGGTGGAGTCTGGGGGAGGCGTGGTCCAGCCTGGGAGGTCCCT
GAGACTCTCCTGTGCAGCCTCTGGATTCGCCTTCAGTAGCTATGGCATGC
ACTGGGTCCGCCAGGCTCCAGGCAAGGGGCTGGAGTGGGTGGCAGTTATA
TCATATGATGGAAATAATAAATACTATGCAGACTCCGTGAAGGGCCGATT
CACCATCTCCAGAGACAATTCCAAGAACACGCTGTATCTGCAAATGAACA
GCCTGAGAGCTGAGGACACGGCTGTGTATTACTGTGCGAGAGATCTAGTG
GTTCGGGGAATTAGGGGGTACTACTACTACTTCGGTATGGACGTCTGGGG
CCAAGGGACCACGGTCACCGTCTCCTCAGCC 3′
(SEQ ID NO: 74)
5′ QLVESGGGVVQPGRSLRLSCAASGFAFSSYGMHWVRQAPGKGLEWVA
VISYDGNNKYYADSVKGRFTISRDNSKNTLYLQMNSLRAEDTAVYYCARD
LVVRGIRGYYYYFGMDVWGQGTTVTVSSA 3′

TABLE 9
2.10.2 Heavy chain V region domains.

| REGION | SEQUENCE | AA RESIDUES* | SEQ ID NO: |
|---|---|---|---|
| FR1 | QLVESGGGVVQPGRSLRLSCAAS | 1-23 | 75 |
| CDR1 | GFAFSSYGMH | 24-33 | 76 |
| FR2 | WVRQAPGKGLEWVA | 34-47 | 77 |
| CDR2 | VISYDGNNKYYADSVKG | 48-64 | 78 |
| FR3 | RFTISRDNSKNTLYLQMNSLRAEDTAVYYCAR | 65-96 | 79 |
| CDR3 | DLVVRGIRGYYYYFGMDV | 97-114 | 80 |
| FR4 | WGQGTTVTVSSA | 115-126 | 81 |

*AA Residues of SEQ ID NO: 74

Antibody 2.10.2 Light Chain Variable Region

(SEQ ID NO: 82)
5′ GATATTGTGATGACTCAGTCTCCACTCTCCCTGCCCGTCACCCCTGG
AGAGCCGGCCTCCATCTCCTGCAGGTCTAGTCAGAGCCTCCTGCATAGTA
ATGGATACAACTATTTGGATTGGTACCTGCAGAAGCCAGGGCAGTCTCCA
CAGCTCCTGATCTATTTGGGTTCTAATCGGGCCTCCGGGGTCCCTGACAG
GTTCAGTGGCAGTGGATCAGGCACAGATTTTACACTGAAAATCAGCAGAG
TGGAGGCTGAGGATGTTGGGGTTTATTACTGCATGCAAGGTCTACAAACT
CCGATCACCTTCGGCCAAGGGACACGACTGGAGATTAAACGA 3′
(SEQ ID NO: 83)
5′ DIVMTQSPLSLPVTPGEPASISCRSSQSLLHSNGYNYLDWYLQKPGQ
SPQLLIYLGSNRASGVPDRFSGSGSGTDFTLKISRVEAEDVGVYYCMQGL
QTPITFGQGTRLEIKR 3′

TABLE 10
2.10.2 Light chain V region domains.

| REGION | SEQUENCE | AA RESIDUES* | SEQ ID NO: |
|---|---|---|---|
| FR1 | DIVMTQSPLSLPVTPGEPASISC | 1-23 | 84 |
| CDR1 | RSSQSLLHSNGYNYLD | 24-39 | 85 |
| FR2 | WYLQKPGQSPQLLIY | 40-54 | 86 |
| CDR2 | LGSNRAS | 55-61 | 87 |
| FR3 | GVPDRFSGSGSGTDFTLKISRVEAEDVGVYYC | 62-93 | 88 |
| CDR3 | MQGLQTPIT | 94-102 | 89 |
| FR4 | FGQGTRLEIKR | 103-113 | 90 |

*AA Residues of SEQ ID NO: 83

Antibody 2.15.1 Heavy Chain Variable Region

(SEQ ID NO: 91)
5′ CAGGTGCAGCTGGTGGAGTCTGGGGGAGGCGTGGTCCAGCCTGGGAG
GTCCCTGAGACTCTCCTGTGCAGCGTCTGGATTCACCTTCAGTAACTATG
GCATTCACTGGGTCCGCCAGGCTCCAGGCAAGGGGCTGGAGTGGGTGGCA
GTTATATGGTTTGATGGACGTAATAAATACTATGCAGACTCCGTGAAGGG
CCGATTCACCATCTCCAGAGACAATTCCAAGAACACGCTGTATCTGCAAA
TGAACAGCCTGAGAGCCGAGGACGCGGCTGTGTATTACTGTGCGAGAGAT
CCCTTTGACTATGGTGACTCCTTCTTTGACTACTGGGGCCAGGGCACCCT
GGTCACCGTCTCCTCAGCC 3′
(SEQ ID NO: 92)
5′ QVQLVESGGGVVQPGRSLRLSCAASGFTFSNYGIHWVRQAPGKGLEW
VAVIWFDGRNKYYADSVKGRFTISRDNSKNTLYLQMNSLRAEDAAVYYCA
RDPFDYGDSFFDYWGQGTLVTVSSA 3′

TABLE 11
2.15.1 Heavy chain V region domains.

| REGION | SEQUENCE | AA RESIDUES* | SEQ ID NO: |
|---|---|---|---|
| FR1 | QVQLVESGGGVVQPGRSLRLSCAAS | 1-25 | 93 |
| CDR1 | GFTFSNYGIH | 26-35 | 94 |
| FR2 | WVRQAPGKGLEWVA | 36-49 | 95 |
| CDR2 | VIWFDGRNKYYADSVKG | 50-66 | 96 |
| FR3 | RFTISRDNSKNTLYLQMNSLRAEDAAVYYCAR | 67-98 | 97 |
| CDR3 | DPFDYGDSFFDY | 99-110 | 98 |
| FR4 | WGQGTLVTVSSA | 111-122 | 99 |

*AA Residues of SEQ ID NO: 92

Antibody 2.15.1 Light Chain Variable Region

(SEQ ID NO: 100)
5′ CTGACTCAGTCTCCATCCTCCCTGTCTGCATCTGTAAGAGACAGAGT
CACCATCACTTGCCGGGCGAGTCAGGACATTAGCAATTATTTAGCCTGGT
ATCAGCAGAAACCAGGGAAAGTTCCTAATCTCCTGATCTATGCTGCATCC
ACTTTGCAATCAGGGGTCCCATCTCGGTTCAGTGGCAGTGGATCTGGGAC
AGATTTCACTCTCACCATCAGCAGCCTGCAGCCTGAAGATGTTGCAACTT
ATTACTGTCAAAAGTATAACAGTGCCCCGCTCACTTTCGGCGGAGGGACC
AAGGTGGAGATCAAACGA 3′
(SEQ ID NO: 101)
5′ LTQSPSSLSASVRDRVTITCRASQDISNYLAWYQQKPGKVPNLLIYA
ASTLQSGVPSRFSGSGSGTDFTLTISSLQPEDVATYYCQKYNSAPLTFGG
GTKVEIKR 3′

TABLE 12
2.15.1 Light chain V region domains.

| REGION | SEQUENCE | AA RESIDUES* | SEQ ID NO: |
|---|---|---|---|
| FR1 | LTQSPSSLSASVRDRVTITC | 1-20 | 102 |
| CDR1 | RASQDISNYLA | 21-31 | 103 |
| FR2 | WYQQKPGKVPNLLIY | 32-46 | 104 |
| CDR2 | AASTLQ | 47-52 | 105 |
| FR3 | GVPSRFSGSGSGTDFTLTISSLQPEDVATYYC | 53-84 | 106 |
| CDR3 | QKYNSAPLT | 85-93 | 107 |
| FR4 | FGGGTKVEIKR | 94-104 | 108 |

*AA Residues of SEQ ID NO: 101

Antibody 2.16.1 Heavy Chain Variable Region

(SEQ ID NO: 109)
5′ CAGGTGCAGCTGGTGGAGTCTGGGGGAGGCTTGGTCAAGCCTGGAGG
GTCCCTGAGACTCTCCTGTGCAGCCTCTGGATTCACCTTCAGTGACTACT
ACATGACCTGGATCCGCCAGGCTCCAGGGAAGGGGCTGGAGTGGGTTTCA
TACATTAGTATTAGTGGTAGTATCACACACTACGCAGACTCAGTGAAGGG
CCGATTCACCATGTCCAGGGACAACGCCAAGAACTCACTGTATCTGCAAA
TGAACAGCCTGAGAGCCGAGGACACGGCCGTGTATTACTGTGCGAGAGAC
GGAGCAGCAGCTGGTACGGATGCTTTTGATATCTGGGGCCACGGGACAAA
GGTCACCGTCTCTTCAGCC 3′
(SEQ ID NO: 110)
5′ QVQLVESGGGLVKPGGSLRLSCAASGFTFSDYYMTWIRQAPGKGLEW
VSYISISGSITHYADSVKGRFTMSRDNAKNSLYLQMNSLRAEDTAVYYCA
RDGAAAGTDAFDIWGHGTKVTVSSA 3′

TABLE 13
2.16.1 Heavy chain V region domains.

| REGION | SEQUENCE | AA RESIDUES* | SEQ ID NO: |
|---|---|---|---|
| FR1 | QVQLVESGGGLVKPGGSLRLSCAAS | 1-25 | 111 |
| CDR1 | GFTFSDYYMT | 26-35 | 112 |
| FR2 | WIRQAPGKGLEWVS | 36-49 | 113 |
| CDR2 | YISISGSITHYADSVKG | 50-66 | 114 |
| FR3 | RFTMSRDNAKNSLYLQMNSLRAEDTAVYYCAR | 67-98 | 115 |
| CDR3 | DGAAAGTDAFDI | 99-110 | 116 |
| FR4 | WGHGTKVTVSSA | 111-122 | 117 |

*AA Residues of SEQ ID NO: 110

Antibody 2.16.1 Light Chain Variable Region

(SEQ ID NO: 118)
5′ GAGATAGTGATGACGCAGTCTCCAGCCACCCTATCTGTGTCTCCAGG
GGACAGAGCCACCCTCTCCTGCAGGGCCAGTCAGAATGTTAGCAGCAACT
TGGCCTGGTACCAGCAGAAACCTGGCCAGGCTCCCAGGCTCCTCATCTTT
GGTGCATCCACCAGGGCCACTGGTATCCCAGCCAGGTTCAGTGGCAGTGG
GTCTGGGACAGAGTTCACTCTCACCATCAGCAGCCTACAGTCTGAAGATT
TTGCAGTTTATTACTGTCAGCAGTATCATTACTGGCCCACTTTCGGCCCT
GGGACCAAAGTGGATATCAAACGA 3′
(SEQ ID NO: 119)
5′ EIVMTQSPATLSVSPGDRATLSCRASQNVSSNLAWYQQKPGQAPRLL
IFGASTRATGIPARFSGSGSGTEFTLTISSLQSEDFAVYYCQQYHYWPTF
GPGTKVDIKR 3′

TABLE 14
2.16.1 Light chain V region domains.

| REGION | SEQUENCE | AA RESIDUES* | SEQ ID NO: |
|---|---|---|---|
| FR1 | EIVMTQSPATLSVSPGDRATLSC | 1-23 | 120 |
| CDR1 | RASQNVSSNLA | 24-34 | 121 |
| FR2 | WYQQKPGQAPRLLIF | 35-49 | 122 |
| CDR2 | GASTRAT | 50-56 | 123 |
| FR3 | GIPARFSGSGSGTEFTLTISSLQSEDFAVYYC | 57-88 | 124 |
| CDR3 | QQYHYWPT | 89-96 | 125 |
| FR4 | FGPGTKVDIKR | 97-107 | 126 |

*AA Residues of SEQ ID NO: 119

Antibody 2.17.1 Heavy Chain Variable Region

(SEQ ID NO: 127)
5′ CAGCTGGTGCAGTCTGGGGCTGAGGTGAAGAAGCCTGGGGCCTCAGT
GAAGGTCTCCTGCAAGGCTTCTGGATACACCTTCACCGGCTTCTATATGC
ACTGGGTGCGACAGACCCCTGGACAAGGGCTTGAGTGGATGGGATGGATC
AACCCTAACAGTGGTGGCACATATTATGTACAGAAGTTTCAGGGCAGGGT
CACCATGACCAGGGACACGTCCATCAGCACAGTCTACATGGAGCTGAGCA
GGTTGAGATCTGACGACACGGCCGTATATTACTGTGCGAGAGATGGGTAT
AGCAGTGGAGAGGACTGGTTCGACCCCTGGGGCCAGGGAACCCTGGTCAC
CGTCTCCTCAGCC 3′
(SEQ ID NO: 128)
5′ QLVQSGAEVKKPGASVKVSCKASGYTFTGFYMHWVRQTPGQGLEWMG
WINPNSGGTYYVQKFQGRVTMTRDTSISTVYMELSRLRSDDTAVYYCARD
GYSSGEDWFDPWGQGTLVTVSSA 3′

TABLE 15
2.17.1 Heavy chain V region domains.

| REGION | SEQUENCE | AA RESIDUES* | SEQ ID NO: |
|---|---|---|---|
| FR1 | QLVQSGAEVKKPGASVKVSCKAS | 1-23 | 129 |
| CDR1 | GYTFTGFYMH | 24-33 | 130 |
| FR2 | WVRQTPGQGLEWMG | 34-47 | 131 |
| CDR2 | WINPNSGGTYYVQKFQG | 48-64 | 132 |
| FR3 | RVTMTRDTSISTVYMELSRLRSDDTAVYYCAR | 65-96 | 133 |
| CDR3 | DGYSSGEDWFDP | 97-108 | 134 |
| FR4 | WGQGTLVTVSSA | 109-120 | 135 |

*AA Residues of SEQ ID NO: 128

Antibody 2.17.1 Light Chain Variable Region

(SEQ ID NO: 136)
5′ GATATTGTGATGACCCAGACTCCACTCTCTCTGTCCGTCACCCCTGG
ACAGCCGGCCTCCATCTCCTGCAAGTCTAGTCAGAGCCTCCTGCATAGTG
GTGGAAAGACCTATTTGTATTGGTACCTGCAGAGGCCAGGCCAGCCTCCA
CAGCTCCTGATCTATGAAGTTTCCAACCGGTTCTCTGGAGTGCCAGATAG
GTTCAGTGGCAGCGGGTCAGGGACAGATTTCACACTGAAAATCAGCCGGG
TGGAGGCTGAGGATGTTGGGGTTTATTACTGCATGCAAAGTATACACCTT
CCGCTCACTTTCGGCGGAGGGACCAAGGTGGAGATCAAACGA 3′
(SEQ ID NO: 137)
5′ DIVMTQTPLSLSVTPGQPASISCKSSQSLLHSGGKTYLYWYLQRPGQ
PPQLLIYEVSNRFSGVPDRFSGSGSGTDFTLKISRVEAEDVGVYYCMQSI
HLPLTFGGGTKVEIKR 3′

TABLE 16
2.17.1 Light chain V region domains.

| REGION | SEQUENCE | AA RESIDUES* | SEQ ID NO: |
|---|---|---|---|
| FR1 | DIVMTQTPLSLSVTPGQPASISC | 1-23 | 138 |
| CDR1 | KSSQSLLHSGGKTYLY | 24-39 | 139 |
| FR2 | WYLQRPGQPPQLLIY | 40-54 | 140 |
| CDR2 | EVSNRFS | 55-61 | 141 |
| FR3 | GVPDRFSGSGSGTDFTLKISRVEAEDVGVYYC | 62-93 | 142 |
| CDR3 | MQSIHLPLT | 94-102 | 143 |
| FR4 | FGGGTKVEIKR | 103-113 | 144 |

*AA Residues of SEQ ID NO: 137

Antibody 2.21.1 Heavy Chain Variable Region

(SEQ ID NO: 145)
5′ CAGGTGCAGCTGGAGCAGTCGGGGGGAGGCCTGGTCAAGCCTGGGGG
GTCCCTGAGATTCTCCTGTGCAGCCTCTGGATTCACCTTCAGTAGCTATA
GCATGAACTGGGTCCGCCAGGCTCCAGGGAAGGGGCTGGAGTGGGTCTCA
TTCATTAGTAGTAGTAGTAGTTACATATACTACGCAGACTCAGTGAAGGG
CCGATTCACCATCTCCAGAGACAACGCCAAGAACTCACTGTATCTGCAAA
TGAACAGCCTGAGAGCCGAGGACACGGCTGTGTATTACTGTGCGAGAGAG
GACTGGGTGGGAGCTACCTTTGACTACTGGGGCCAGGGAACCCTGGTCAC
CGTCTCCTCAGCC 3′
(SEQ ID NO: 146)
5′ QVQLEQSGGGLVKPGGSLRFSCAASGFTFSSYSMNWVRQAPGKGLEW
VSFISSSSSYIYYADSVKGRFTISRDNAKNSLYLQMNSLRAEDTAVYYCA
REDWVGATFDYWGQGTLVTVSSA 3′

TABLE 17
2.21.1 Heavy chain V region domains.

| REGION | SEQUENCE | AA RESIDUES* | SEQ ID NO: |
|---|---|---|---|
| FR1 | QVQLEQSGGGLVKPGGSLRFSCAAS | 1-25 | 147 |
| CDR1 | GFTFSSYSMN | 26-35 | 148 |
| FR2 | WVRQAPGKGLEWVS | 36-49 | 149 |
| CDR2 | FISSSSSYIYYADSVKG | 50-66 | 150 |
| FR3 | RFTISRDNAKNSLYLQMNSLRAEDTAVYYCAR | 67-98 | 151 |
| CDR3 | EDWVGATFDY | 99-108 | 152 |
| FR4 | WGQGTLVTVSSA | 109-120 | 153 |

*AA Residues of SEQ ID NO: 146

Antibody 2.21.1 Light Chain Variable Region

(SEQ ID NO: 154)
5′ GACATTCAGCTGACCCAGTCTCCATCCTCCCTGTCTGCATCTGTAGG
AGACAGAGTCACCATCACTTGTCGGGCGAGTCAGGGCATTAGGAATTATT
TAGCCTGGTATCAGCAGAAACCAGGGAAAGTTCCTAAGCTCCTGATCTAT
GCTGCTTCCGCTTTGAAATTAGGGGTCCCATCTCGGTTCAGTGGCAGTGG
ATCTGGGACAGATTTCACTCTCACCATCAGCAGCCTGCAGCCTGAAGATG
TTGCAACTTATTACTGTCAAAAGTATAACAGTGCCCCGATCACCTTCGGC
CAAGGGACACGACTGGACATTAAACGA 3′
(SEQ ID NO: 155)
5′ DIQLTQSPSSLSASVGDRVTITCRASQGIRNYLAWYQQKPGKVPKLL
IYAASALKLGVPSRFSGSGSGTDFTLTISSLQPEDVATYYCQKYNSAPIT
FGQGTRLDIKR 3′

TABLE 18
2.21.1 Light chain V region domains.

| REGION | SEQUENCE | AA RESIDUES* | SEQ ID NO: |
|---|---|---|---|
| FR1 | DIQLTQSPSSLSASVGDRVTITC | 1-23 | 156 |
| CDR1 | RASQGIRNYLA | 24-34 | 157 |
| FR2 | WYQQKPGKVPKLLIY | 35-49 | 158 |
| CDR2 | AASALKL | 50-56 | 159 |
| FR3 | GVPSRFSGSGSGTDFTLTISSLQPEDVATYYC | 57-88 | 160 |
| CDR3 | QKYNSAPIT | 89-97 | 161 |
| FR4 | FGQGTRLDIKR | 98-108 | 162 |

*AA Residues of SEQ ID NO: 155

Antibody 2.22.1 Heavy Chain Variable Region

(SEQ ID NO: 163)
5′ CAGGTGCAGCTGGAGCAGTCGGGCCCAGGACTGGTGAAGCCTTCACA
GAACCTGTCCCTCACCTGCACTGTCTCTGGTGGCTCCATCAGCAGTGGTG
GTTATTTCTGGAGCTGGATCCGCCAGCACCCAGGGAAGGGCCTGGAGTGG
ATTGGGTACATCTATTACAGTGGGAACACCTACTACAACCCGTCCCTCAA
GAGTCGAGTTACCATATCAGTTGACACGTCTAAGAACCAGTTCTCCCTGA
AACTGAGCTCTGTGACTGCCGCGGACACGGCCGTGTATTACTGTGCGAGA
GACTATTACTATGATACTAGTGGTTTTTCCTACCGTTACGACTGGTACTA
CGGTATGGACGTCTGGGGCCAAGGGACCACGGTCACCGTCTCCTCAGC
C 3′
(SEQ ID NO: 164)
5′ QVQLEQSGPGLVKPSQNLSLTCTVSGGSISSGGYFWSWIRQHPGKGL
EWIGYIYYSGNTYYNPSLKSRVTISVDTSKNQFSLKLSSVTAADTAVYYC
ARDYYYDTSGFSYRYDWYYGMDVWGQGTTVTVSSA 3′

TABLE 19
2.22.1 Heavy chain V region domains.

| REGION | SEQUENCE | AA RESIDUES* | SEQ ID NO: |
|---|---|---|---|
| FR1 | QVQLEQSGPGLVKPSQNLSLTCTVS | 1-25 | 165 |
| CDR1 | GGSISSGGYFWS | 26-37 | 166 |
| FR2 | WIRQHPGKGLEWIG | 38-51 | 167 |
| CDR2 | YIYYSGNTYYNPSLKS | 52-67 | 168 |
| FR3 | RVTISVDTSKNQFSLKLSSVTAADTAVYYCAR | 68-99 | 169 |
| CDR3 | DYYYDTSGFSYRYDWYYGMDV | 100-120 | 170 |
| FR4 | WGQGTTVTVSSA | 121-132 | 171 |

*AA Residues of SEQ ID NO: 164

Antibody 2.22.1 Light Chain Variable Region

(SEQ ID NO: 172)
5′ GACATCCAGCTGACCCAGTCTCCATCCTCCCTGTCTGCATCTGTAGG
AGACAGAGTCACCATCACTTGCCGGGCAAGTCAGGGCATTAGAAATGATT
TAGGCTGGTATCAGCAGAAACCAGGGAAAGCCCCTAAGCGCCTGATCTAT
GCTGCATCCAGTTTGCAAAATGGGGTCCCATCAAGGTTCAGCGGCAGTGG
ATCTGGGACAGAATTCACTCTCACAATCAGCAGCCTGCAGCCTGAAGATT
TTGCAACTTATTACTGTCTACAACATAATACTTACCCGGCGTTCGGCCAA
GGGACCAAGGTGGAAATCAAACGA 3′
(SEQ ID NO: 173)
5′ DIQLTQSPSSLSASVGDRVTITCRASQGIRNDLGWYQQKPGKAPKRL
IYAASSLQNGVPSRFSGSGSGTEFTLTISSLQPEDFATYYCLQHNTYPAF
GQGTKVEIKR 3′

TABLE 20
2.22.1 Light chain V region domains.

| REGION | SEQUENCE | AA RESIDUES* | SEQ ID NO: |
|---|---|---|---|
| FR1 | DIQLTQSPSSLSASVGDRVTITC | 1-23 | 174 |
| CDR1 | RASQGIRNDLG | 24-34 | 175 |
| FR2 | WYQQKPGKAPKRLIY | 35-49 | 176 |
| CDR2 | AASSLQN | 50-56 | 177 |
| FR3 | GVPSRFSGSGSGTEFTLTISSLQPEDFATYYC | 57-88 | 178 |
| CDR3 | LQHNTYPA | 89-97 | 179 |
| FR4 | FGQGTKVEIKR | 98-108 | 180 |

*AA Residues of SEQ ID NO: 173

Antibody 2.24.1 Heavy Chain Variable Region

(SEQ ID NO: 181)
5′ CAGCTGGTGCAGTCTGGAGCAGAAGTGAAAAAGCCCGGGGAGTCTCT
GAAGATCTCCTGTCAGGGTTCTGGATACATCTTTACCAACTACTGGATCG
GCTGGGTGCGCCAGATGCCCGGGAAAGGCCTGGAGTGGATGGGGGTCATC
TATCCTGATGACTCTGATACCAGATACAGCCCGTCCTTCCAAGGCCAGGT
CACCATCTCAGCCGACAAGTCCATCAGCACCGCCTACCTGCAGTGGAGCA
GCCTGAAGGCCTCGGACACCGCCATATATTACTGTGCGAGACAAAAATGG
CTACAACACCCCTTTGACTACTGGGGCCAGGGAACCCTGGTCACCGTCTC
CTCAGCC 3′
(SEQ ID NO: 182)
5′ QLVQSGAEVKKPGESLKISCQGSGYIFTNYWIGWVRQMPGKGLEWMG
VIYPDDSDTRYSPSFQGQVTISADKSISTAYLQWSSLKASDTAIYYCARQ
KWLQHPFDYWGQGTLVTVSSA 3′

TABLE 21
2.24.1 Heavy chain V region domains.

| REGION | SEQUENCE | AA RESIDUES* | SEQ ID NO: |
|---|---|---|---|
| FR1 | QLVQSGAEVKKPGESLKISCQGS | 1-23 | 183 |
| CDR1 | GYIFTNYWIG | 24-33 | 184 |
| FR2 | WVRQMPGKGLEWMG | 34-47 | 185 |
| CDR2 | VIYPDDSDTRYSPSFQG | 48-64 | 186 |
| FR3 | QVTISADKSISTAYLQWSSLKASDTAIYYCAR | 65-96 | 187 |
| CDR3 | QKWLQHPFDY | 97-106 | 188 |
| FR4 | WGQGTLVTVSSA | 107-118 | 189 |

*AA Residues of SEQ ID NO: 182

Antibody 2.24.1 Light Chain Variable Region

(SEQ ID NO: 190)
5′ GAAATTGTGTTGACGCAGTCACCAGGCACCCTGTCTTTGTCTCCAGG
GGAAAGAGTCACCCTCTCATGCAGGGCCAGTCAGAGTGTTAGCAGCAGAT
ACTTAGCCTGGTACCAGCAGAAACCTGGCCAGGCTCCCAGGCTCCTCATC
TATGGTGCATCCAGCAGGGCCACTGGCATCCCAGACAGGTTCAGTGGCAG
TGGGTCTGGGACAGACTTCACTCTCACCATCAGCAGACTGGAGCCTGAAG
ATTTTGCAGTTTATTACTGTCAGCAGTATGGTAGCTCACCTCGGACGTTC
GGCCAAGGGACCAAGGTGGAAATCAAACGA 3′
(SEQ ID NO: 191)
5′ EIVLTQSPGTLSLSPGERVTLSCRASQSVSSRYLAWYQQKPGQAPRL
LIYGASSRATGIPDRFSGSGSGTDFTLTISRLEPEDFAVYYCQQYGSSPR
TFGQGTKVEIKR 3′

TABLE 22
2.24.1 Light chain V region domains.

| REGION | SEQUENCE | AA RESIDUES* | SEQ ID NO: |
|---|---|---|---|
| FR1 | EIVLTQSPGTLSLSPGERVTLSC | 1-23 | 192 |
| CDR1 | RASQSVSSRYLA | 24-35 | 193 |
| FR2 | WYQQKPGQAPRLLIY | 36-50 | 194 |
| CDR2 | GASSRAT | 51-57 | 195 |
| FR3 | GIPDRFSGSGSGTDFTLTISRLEPEDFAVYY | 58-88 | 196 |
| CDR3 | QQYGSSPRT | 89-97 | 197 |
| FR4 | FGQGTKVEIKR | 98-109 | 198 |

*AA Residues of SEQ ID NO: 191

Antibody 2.3.1 Heavy Chain Variable Region

(SEQ ID NO: 199)
5′ CAGGTGCAGCTGGTGCAGTCTGGGGCTGAGGTGAAGAAGCCTGGGGC
CTCAGTGAAGGTCTCCTGCAAGGCTTCTGGATACACCTTCACCGGCTACT
ATATGCACTGGGTGCGACAGGCCCCTGGACAAGGGCTTGAGTGGATGGGA
TGGATCAACCCTAACAGTGGTGGCACAAACTATGCACAGAAGTTTCAGGA
CAGGGTCACCATGACCAGGGACACGTCCATCAGCACAGCCTACATGGAGC
TGAGCAGGCTGAGATCTGACGACACGGCCGTGTATTACTGTGCGAGAGAT
TTCTTTGGTTCGGGGAGTCTCCTCTACTTTGACTACTGGGGCCAGGGAAC
CCTGGTCACCGTCTCCTCAGCC 3′
(SEQ ID NO: 200)
5′ QVQLVQSGAEVKKPGASVKVSCKASGYTFTGYYMHWVRQAPGQGLEW
MGWINPNSGGTNYAQKFQDRVTMTRDTSISTAYMELSRLRSDDTAVYYCA
RDFFGSGSLLYFDYWGQGTLVTVSSA 3′

TABLE 23
2.3.1 Heavy chain V region domains.

| REGION | SEQUENCE | AA RESIDUES* | SEQ ID NO: |
|---|---|---|---|
| FR1 | QVQLVQSGAEVKKPGASVKVSCKAS | 1-25 | 201 |
| CDR1 | GYTFTGYYMH | 26-35 | 202 |
| FR2 | WVRQAPGQGLEWMG | 36-49 | 203 |
| CDR2 | WINPNSGGTNYAQKFQD | 50-66 | 204 |
| FR3 | RVTMTRDTSISTAYMELSRLRSDDTAVYYCAR | 67-98 | 205 |
| CDR3 | DFFGSGSLLYFDY | 99-111 | 206 |
| FR4 | WGQGTLVTVSSA | 112-123 | 207 |

*AA Residues of SEQ ID NO: 200

Antibody 2.3.1 Light Chain Variable Region

(SEQ ID NO: 208)
5′ GATATTGTGATGACCCAGACTCCACTCTCTCTGTCCGTCACCCCTGGACAGCCGGCCTCCAT
CTCCTGCAAGTCTAGTCAGAGCCTCCTGCATAGTGGTGGAAAGACCTATTTGTATTGGTACCTGC
AGAGGCCAGGCCAGCCTCCACAGCTCCTGATCTATGAAGTTTCCAACCGGTTCTCTGGAGTGCCA
GATAGGTTCAGTGGCAGCGGGTCAGGGACAGATTTCACACTGAAAATCAGCCGGGTGGAGGCTGA
GGATGTTGGGGTTTATTACTGCATGCAAAGTATACACCTTCCGCTCACTTTCGGCGGAGGGACCA
AGGTGGAGATCAAACGA 3′
(SEQ ID NO: 209)
5′ DIVMTQTPLSLSVTPGQPASISCKSSQSLLHSGGKTYLYWYLQRPGQPPQLLIYEVSNRFSG
VPDRFSGSGSGTDFTLKISRVEAEDVGVYYCMQSIHLPLTFGGGTKVEIKR 3′

TABLE 24
2.3.1 Light chain V region domains.

| REGION | SEQUENCE | AA RESIDUES* | SEQ ID NO: |
|---|---|---|---|
| FRI | DIVMTQTPLSLSVTPGQPASISC | 1-23 | 210 |
| CDR1 | KSSQSLLHSGGKTYLY | 24-39 | 211 |
| FR2 | WYLQRPGQPPQLLIY | 40-54 | 212 |
| CDR2 | EVSNRFS | 55-61 | 213 |
| FR3 | GVPDRFSGSGSGTDFTLKISRVEAEDVGVYYC | 62-93 | 214 |
| CDR3 | MQSIHLPLT | 94-102 | 215 |
| FR4 | FGGGTKVEIKR | 103-113 | 216 |

*AA Residues of SEQ ID NO: 209

Antibody 2.7.1 Heavy Chain Variable Region

(SEQ ID NO: 217)
5′ CAGGTGCAGCTGGAGCAGTCGGGGGGAGGCGTGGTCCAGCCTGGGAGGTCCCTGAGACTCTC
CTGTGCAGCGTCTGGATTCACCTTCAATAACTATGGCATGCACTGGGTCCGCCAGGCTCCAGGCA
AGGGGCTGGAGTGGGTGGCAGTTATATGGTATGATGGAAGTAATAAATACTATGCAGACTCCGTG
AAGGGCCGATTCACCATCTCCAGAGACAATTCCAAGAACACGCTGTATCTGCAAATGAACAGCCT
GAGAGCCGAGGACACGGCTGTGTATTACTGTGCGAAAGATGAGGAATACTACTATGTTTCGGGGC
TTGACTACTGGGGCCAGGGAACCCTGGTCACCGTCTCCTCAGCC 3′
(SEQ ID NO: 218)
5′ QVQLEQSGGGVVQPGRSLRLSCAASGFTFNNYGMHWVRQAPGKGLEWVAVIWYDGSNKYYAD
SVKGRFTISRDNSKNTLYLQMNSLRAEDTAVYYCAKDEEYYYVSGLDYWGQGTLVTVSSA 3′

TABLE 25
2.7.1 Heavy chain V region domains.

| REGION | SEQUENCE | AA RESIDUES* | SEQ ID NO: |
|---|---|---|---|
| FRI | QVQLEQSGGGVVQPGRSLRLSCAAS | 1-25 | 219 |
| CDR1 | GFTFNNYGMH | 26-35 | 220 |
| FR2 | WVRQAPGKGLEWVA | 36-49 | 221 |
| CDR2 | VIWYDGSNKYYADSVKG | 50-66 | 222 |
| FR3 | RFTISRDNSKNTLYLQMNSLRAEDTAVYYCAK | 67-98 | 223 |
| CDR3 | DEEYYYVSGLDY | 99-110 | 224 |
| FR4 | WGQGTLVTVSSA | 111-122 | 225 |

*AA Residues of SEQ ID NO: 218

Antibody 2.7.1 Light Chain Variable Region

(SEQ ID NO: 226)
5′ CTGACTCAGTCTCCATCCTCCCTGTCTGCATCTGTAAGAGACAGAGTCACCATCACTTGCCG
GGCGAGTCAGGACATTAGCAATTATTTAGCCTGGTATCAGCAGAAACCAGGGAAAGTTCCTAATC
TCCTGATCTATGCTGCATCCACTTTGCAATCAGGGGTCCCATCTCGGTTCAGTGGCAGTGGATCT
GGGACAGATTTCACTCTCACCATCAGCAGCCTGCAGCCTGAAGATGTTGCAACTTATTACTGTCA
AAAGTATAACAGTGCCCCGCTCACTTTCGGCGGAGGGACCAAGGTGGAGATCAAACGA 3′
(SEQ ID NO: 227)
5′ LTQSPSSLSASVRDRVTITCRASQDISNYLAWYQQKPGKVPNLLIYAASTLQSGVPSRFSGS
GSGTDFTLTISSLQPEDVATYYCQKYNSAPLTFGGGTKVEIKR 3′

TABLE 26
2.7.1 Light chain V region domains.

| REGION | SEQUENCE | AA RESIDUES* | SEQ ID NO: |
|---|---|---|---|
| FRI | LTQSPSSLSASVRDRVTITC | 1-20 | 228 |
| CDR1 | RASQDISNYLA | 21-31 | 229 |
| FR2 | WYQQKPGKVPNLLIY | 32-46 | 230 |
| CDR2 | AASTLQ | 47-52 | 231 |
| FR3 | GVPSRFSGSGSGTDFTLTISSLQPEDVATYYC | 53-84 | 232 |
| CDR3 | QKYNSAPLT | 85-93 | 233 |
| FR4 | FGGGTKVEIKR | 94-104 | 234 |

*AA Residues of SEQ ID NO: 227

Antibody 2.8.1 Heavy Chain Variable Region

(SEQ ID NO: 235)
5′ CAGATCACCTTGAAGGAGTCTGGTCCTACGCTGGTGACACCCACACAGACCCTCACGCTGAC
CTGCACCTTCTCTGGGTTCTCACTCAGCACTGGTGGAATGGGTGTGGGCTGGATCCGTCAGCCCC
CAGGAAAGGCCCTGGACTGGCTTACACTCATTTATTGGAATGATGATAAGCACTACAGCCCATCT
CTGAAGAGCAGGCTTACCATCACCAAGGACACCTCCAAAAACCAGGTGGTCCTTAGAATGACCAA
CATGGACCCTGTGGACACAGCCACTTATTACTGTGCACACCTGCATTACGATATTTTGACTGGTT
TTAACTTTGACTACTGGGGCCAGGGAACCCTGGTCACCGTCTCCTCAGCC 3′
(SEQ ID NO: 236)
5′ QITLKESGPTLVTPTQTLTLTCTFSGFSLSTGGMGVGWIRQPPGKALDWLTLIYWNDDKHYS
PSLKSRLTITKDTSKNQVVLRMTNMDPVDTATYYCAHLHYDILTGFNFDYWGQGTLVTVSSA 3′

TABLE 27
2.8.1 Heavy chain V region domains.

| REGION | SEQUENCE | AA RESIDUES* | SEQ ID NO: |
|---|---|---|---|
| FRI | QITLKESGPTLVTPTQTLTLTCTFS | 1-25 | 237 |
| CDR1 | GFSLSTGGMGVG | 26-37 | 238 |
| FR2 | WIRQPPGKALDWLT | 38-51 | 239 |
| CDR2 | LIYWNDDKHYSPSLKS | 52-67 | 240 |
| FR3 | RLTITKDTSKNQVVLRMTNMDPVDTATYYCAH | 68-99 | 241 |
| CDR3 | LHYDILTGFNFDY | 100-112 | 242 |
| FR4 | WGQGTLVTVSSA | 113-124 | 243 |

*AA Residues of SEQ ID NO: 236

Antibody 2.8.1 Light Chain Variable Region

(SEQ ID NO: 244)
5′ GATATTGTGATGACCCAGACTCCACTCTCCCTGCCCGTCACCCCTGGAGAGCCGGCCTCCAT
CTCCTGCAGGTCTAGTCAGAGCCTCTTGGATAGTGATGATGGAAACACCTATTTGGACTGGTACC
TGCAGAAGCCAGGGCAGTCTCCACAGCTCCTGATCTATACGCTTTCCTATCGGGCCTCTGGAGTC
CCAGACAGGTTCAGTGGCAGTGGGTCAGGCACTGATTTCACACTGAAAATCAGCAGGGTGGAGGC
TGAGGATGTTGGAGTTTATTACTGCATGCAACGTATAGAGTTTCCGCTCACTTTCGGCGGAGGGA
CCAAGGTGGAGATCAAACGA 3′
(SEQ ID NO: 245)
5′ DIVMTQTPLSLPVTPGEPASISCRSSQSLLDSDDGNTYLDWYLQKPGQSPQLLIYTLSYRAS
GVPDRFSGSGSGTDFTLKISRVEAEDVGVYYCMQRIEFPLTFGGGTKVEIKR 3′

TABLE 28
2.8.1 Light chain V region domains.

| REGION | SEQUENCE | AA RESIDUES* | SEQ ID NO: |
|---|---|---|---|
| FRI | DIVMTQTPLSLPVTPGEPASISC | 1-23 | 246 |
| CDR1 | RSSQSLLDSDDGNTYLD | 24-40 | 247 |
| FR2 | WYLQKPGQSPQLLIY | 41-55 | 248 |
| CDR2 | TLSYRAS | 56-62 | 249 |
| FR3 | GVPDRFSGSGSGTDFTLKISRVEAEDVGVYYC | 63-94 | 250 |
| CDR3 | MQRIEFPLT | 95-103 | 251 |
| FR4 | FGGGTKVEIKR | 103-114 | 252 |

*AA Residues of SEQ ID NO: 245

Antibody 2.6.1 Heavy Chain Variable Region

(SEQ ID NO: 253)
5′ CAGGTGCAGCTGGTGCAGTCTGGGGCTGAGGTGAAGAAGCCTGGGGCCTCAGTGAAGGTCTC
CTGCAAGGCTTCTGGATACACCTTCACCGGCTACTATATGCACTGGGTGCGACAGGCCCCTGGAC
AAGGGCTTGAGTGGATGGGATGGATCAACCCTAACAGTGGTGGCACAAACTATGCACAGAAGTTT
CAGGACAGGGTCACCATGACCAGGGACACGTCCATCAGCACAGCCTACATGGAGCTGAGCAGGCT
GAGATCTGACGACACGGCCGTGTATTACTGTGCGAGAGATTTCTTTGGTTCGGGGAGTCTCCTCT
ACTTTGACTACTGGGGCCAGGGAACCCTGGTCACCGTCTCCTCAGCC 3′
(SEQ ID NO: 254)
5′ QVQLVQSGAEVKKPGASVKVSCKASGYTFTGYYMHWVRQAPGQGLEWMGWINPNSGGTNYAQ
KFQDRVTMTRDTSISTAYMELSRLRSDDTAVYYCARDFFGSGSLLYFDYWGQGTLVTVSSA 3′

TABLE 29
2.6.1 Heavy chain V region domains.

| REGION | SEQUENCE | AA RESIDUES* | SEQ ID NO: |
|---|---|---|---|
| FRI | QVQLVQSGAEVKKPGASVKVSCKAS | 1-25 | 255 |
| CDR1 | GYTFTGYYMH | 26-35 | 256 |
| FR2 | WVRQAPGQGLEWMG | 36-49 | 257 |
| CDR2 | WINPNSGGTNYAQKFQD | 50-66 | 258 |
| FR3 | RVTMTRDTSISTAYMELSRLRSDDTAVYYCAR | 67-98 | 259 |
| CDR3 | DFFGSGSLLYFDY | 99-112 | 260 |
| FR4 | WGQGTLVTVSSA | 113-124 | 261 |

*AA Residues of SEQ ID NO: 254

Antibody 2.6.1 Light Chain Variable Region

(SEQ ID NO: 262)
5′ GATATTGTGATGACCCAGACTCCACTCTCTCTGTCCGTCACCCCTGGACAGCCGGCCTCCAT
CTCCTGCAAGTCTAGTCAGAGCCTCCTGCATAGTGGTGGAAAGACCTATTTGTATTGGTACCTGC
AGAGGCCAGGCCAGCCTCCACAGCTCCTGATCTATGAAGTTTCCAACCGGTTCTCTGGAGTGCCA
GATAGGTTCAGTGGCAGCGGGTCAGGGACAGATTTCACACTGAAAATCAGCCGGGTGGAGGCTGA
GGATGTTGGGGTTTATTACTGCATGCAAAGTATACACCTTCCGCTCACTTTCGGCGGAGGGACCA
AGGTGGAGATCAAACGA 3′
(SEQ ID NO: 263)
5′ DIVMTQTPLSLSVTPGQPASISCKSSQSLLHSGGKTYLYWYLQRPGQPPQLLIYEVSNRFSG
VPDRFSGSGSGTDFTLKISRVEAEDVGVYYCMQSIHLPLTFGGGTKVEIKR 3′

TABLE 30
2.6.1 Light chain V region domains.

| REGION | SEQUENCE | AA RESIDUES* | SEQ ID NO: |
|---|---|---|---|
| FRI | DIVMTQTPLSLSVTPGQPASISC | 1-23 | 264 |
| CDR1 | KSSQSLLHSGGKTYLY | 24-39 | 265 |
| FR2 | WYLQRPGQPPQLLIY | 40-54 | 266 |
| CDR2 | EVSNRFS | 55-61 | 267 |
| FR3 | GVPDRFSGSGSGTDFTLKISRVEAEDVGVYYC | 62-93 | 268 |
| CDR3 | MQSIHLPLT | 94-102 | 269 |
| FR4 | FGGGTKVEIKR | 103-113 | 270 |

AA Residues of SEQ ID NO: 263

Anti-GPNMB antibodies include germline human antibody heavy chain V, D, J combinations and light chain V, J combinations including nucleotide and amino acid sequence of the V_{H} and V_{L} domain FR and CDR regions with specificity for GPNMB.

Upon exposure to antigen, those B cells with antigen binding specificity based on germline sequences are activated, proliferate, and differentiate to produce immunoglobulins of different isotypes as well as undergo somatic mutation and/or affinity maturation to produce immunoglobulins of higher affinity for the antigen. The current invention provides the nucleotide and amino acid sequence of such affinity matured V domain FR and CDR regions having specificity to GPNMB.

Fab type antibody fragments containing the antigen binding portion of the antibody molecule may consist of the L chain covalently linked by a disulfide bond to a portion of the H chain which has the V domain and first constant domain. Single chain Fv antibody fragment (scFv) has the H variable domain linked to the L variable domain by a polypeptide linker. The invention provides antibody fragments such as Fab and scFv molecules having sequences derived from germline or affinity matured V domains of antibodies binding specifically to GPNMB.

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments (see, e.g., Songsivilai & Lachmann, 1990 Clin. Exp. Immunol. 79: 315-321; Kostelny et al., 1992 J. Immunol. 148:1547-1553). Bispecific antibodies do not exist in the form of fragments having a single binding site (e.g., Fab, Fab′, and Fv).

It will be appreciated that such bifunctional or bispecific antibodies are contemplated and encompassed by the invention. A bispecific single chain antibody with specificity to GPNMB and to the CD3 antigen on cytotoxic T lymphocytes can be used to direct these T cells to tumor cells expressing GPNMB and cause apoptosis and eradication of the tumor. Bispecific scFv constructs for this purpose are described herein. The scFv components specific for GPNMB can be derived from anti-GPNMB antibodies described herein. In some embodiments, the anti-GPNMB antibody components disclosed herein can be used to generate a biologically active scFv directed against GPNMB. The anti-CD3 scFv component of the therapeutic bispecific scFv was derived from a sequence deposited in Genbank (accession number CAE85148). Alternative antibodies known to target CD3 or other T cell antigens may similarly be effective in treating malignancies when coupled with anti-GPNMB, whether on a single-chain backbone or a full IgG.

GPNMB binding human antibodies may include H or L constant domains including L kappa or lambda constant regions, or any isotype H constant domain. For example, the GPNMB binding human antibodies include germline sequences such as the following germline sequences shown in PCT Publication No. WO2006/071441: the heavy chain V regions: VH1-2, VH2-5, VH3-11, VH3-21, VH3-30, VH3-33, VH4-31, VH4-59 or VH5-51; the heavy chain D region: D1-20, D1-26, D3-10, D3-16, D3-22, D3-9, D4-17, D5-24, D6-13, or D6-19; the heavy chain J region: JH3b, JH4b, JH5b or JH6b; the light chain V kappa regions A2, A3, A20, A27, A30), L2 or O1; and the J region JK1, JK2, JK3), JK4 or JK5). (generally, see Kabat Sequences of Proteins of Immunological Interest, National Institutes of Health, Bethesda, Md. 1987 and 1991; also see Chothia & Lesk 1987 J. Mol. Biol. 196:901-917; Chothia et al. 1989 Nature 342:878-883). In a particular embodiment of the invention, human antibodies with binding specificity to GPNMB are combined germline regions as shown in Table 1 of PCT Publication No. WO2006/071441. In some embodiments, the human anti-GPNMB antibodies are derived from the germline regions set forth above, as described throughout the specification in PCT Publication No. WO2006/071441.

The antibodies of the invention may bind an epitope of GPNMB (SEQ ID NO: 271), preferably within the mature sequence of GPNMB and more preferably within the extracellular domain (ECD) of GPNMB.

Antibodies of the invention may bind GPNMB with an affinity of 10⁻⁶ to 10⁻¹¹, and preferably with an affinity of 10⁻⁷ or greater and even more preferably 10⁻⁸ or greater. In a preferred embodiment, antibodies described herein bind to GPNMB with very high affinities (Kd), for example a human antibody that is capable of binding GPNMB with a Kd less than, but not limited to, 10⁻⁷, 10⁻⁸, 10⁻⁹, 10⁻¹⁰, 10⁻¹¹, 10⁻¹², 10⁻¹³ or 10⁻¹⁴ M, or any range or value therein. Affinity and/or avidity measurements can be measured by KinExA® and/or BIACORE®, as described herein. In particular embodiments antibodies of the invention bind to GPNMB with Kds ranging from 50 to 150 pM.

Epitope mapping and secondary and tertiary structure analyses can be carried out to identify specific 3D structures assumed by the disclosed antibodies and their complexes with antigens (see, e.g., Epitope Mapping Protocols, ed. Morris, Humana Press, 1996). Such methods include, but are not limited to, X-ray crystallography (Biochem. Exp. Biol., 11:7-13, 1974) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

Furthermore, the specific part of the protein immunogen recognized by antibody may be determined by assaying the antibody reactivity to parts of the protein, for example an N terminal and C terminal half. The resulting reactive fragment can then be further dissected, assaying consecutively smaller parts of the immunogen with the antibody until the minimal reactive peptide is defined. Alternatively, the binding specificity, i.e., the epitope, of anti-GPNMB antibodies of the invention may be determined by subjecting GPNMB immunogen to SDS-PAGE either in the absence or presence of a reduction agent and analyzed by immunoblotting. Epitope mapping may also be performed using SELDI. SELDI ProteinChip® (LumiCyte) arrays used to define sites of protein-protein interaction. GPNMB protein antigen or fragments thereof may be specifically captured by antibodies covalently immobilized onto the PROTEINCHIP array surface. The bound antigens may be detected by a laser-induced desorption process and analyzed directly to determine their mass.

The epitope recognized by anti-GPNMB antibodies described herein may be determined by exposing the PROTEINCHIP Array to a combinatorial library of random peptide 12-mer displayed on Filamentous phage (New England Biolabs). Antibody-bound phage are eluted and then amplified and taken through additional binding and amplification cycles to enrich the pool in favor of binding sequences. After three or four rounds, individual binding clones are further tested for binding by phage ELISA assays performed on antibody-coated wells and characterized by specific DNA sequencing of positive clones.

### Antibody Derivatives

Anti-GPNMB antibodies include derivatives of the antibodies described herein. In these derivative antibodies, CDRs are not limited to the specific sequences of H and L variable domains identified, e.g., in Table 1 of WO2006/071441, and may include variants of these sequences that retain the ability to specifically bind GPNMB. Such variants may be derived from the sequences by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity of the antibody for its target. For example, conservative amino acid substitutions can be made within any one of the disclosed CDRs. Various alterations can be made according to the methods described in the art (Antibody Engineering, 2nd ed., Oxford University Press, ed. Borrebaeck, 1995). These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs. Furthermore, any native residue in the polypeptide may also be substituted with alanine (Acta Physiol. Scand. Suppl. 643:55-67, 1998; Adv. Biophys. 35:1-24, 1998).

Derivatives and analogs of antibodies of the invention can be produced by various techniques well known in the art, including recombinant and synthetic methods (Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany).

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in the art (for example, Proteins, Structures and Molecular Principles (Creighton, Ed., W. H. Freeman and Company, New York (1984)).

For example, a method for making an H variable domain which is an amino acid sequence variant of an H variable domain of the invention includes a step of adding, deleting, substituting, or inserting one or more amino acids in the amino acid sequence of the presently disclosed H variable domain, optionally combining the H variable domain thus provided with one or more L variable domains, and testing the H variable domain or H variable/L variable combination or combinations for specific binding to GPNMB and, optionally, testing the ability of such antigen-binding domain to modulate GPNMB activity.

An analogous method can be employed in which one or more sequence variants of an L variable domain disclosed herein are combined with one or more H variable domains.

A further aspect of the disclosure provides a method of preparing antigen-binding fragment that specifically binds with GPNMB. The method comprises: (a) providing a starting repertoire of nucleic acids encoding a H variable domain that either includes a CDR3 to be replaced or lacks a CDR3 encoding region; (b) combining the repertoire with a donor nucleic acid encoding an amino acid sequence substantially as set out herein for a H variable CDR3 such that the donor nucleic acid is inserted into the CDR3 region in the repertoire, so as to provide a product repertoire of nucleic acids encoding a H variable domain; (c) expressing the nucleic acids of the product repertoire; (d) selecting a binding fragment specific for GPNMB; and (e) recovering the specific binding fragment or nucleic acid encoding it.

Again, an analogous method may be employed in which a L variable CDR3 of the invention is combined with a repertoire of nucleic acids encoding a L variable domain, which either include a CDR3 to be replaced or lack a CDR3 encoding region. The donor nucleic acid may be selected from nucleic acids encoding an amino acid sequence substantially as set out in SEQ ID NOs: 2, 11, 20, 29, 38, 47, 56, 65, 74, 83, 92, 101, 110, 119, 128, 137, 146, 155, 164, 173, 182, 191, 200, 209, 218, 227, 236, 245, 254 and 263. A sequence encoding a CDR of the invention (e.g., CDR3) may be introduced into a repertoire of variable domains lacking the respective CDR (e.g., CDR3), using recombinant DNA technology, for example, using methodology described by Marks et al. (Bio/Technology (1992) 10: 779-783). In particular, consensus primers directed at or adjacent to the 5′ end of the variable domain area can be used in conjunction with consensus primers to the third framework region of human H variable genes to provide a repertoire of H variable domains lacking a CDR3. The repertoire may be combined with a CDR3 of a particular antibody. Using analogous techniques, the CDR3-derived sequences may be shuffled with repertoires of H variable or L variable domains lacking a CDR3, and the shuffled complete H variable or L variable domains combined with a cognate L variable or H variable domain to make the GPNMB specific antibodies of the invention. The repertoire may then be displayed in a suitable host system such as the phage display system such as described in WO92/01047 so that suitable antigen-binding fragments can be selected.

Analogous shuffling or combinatorial techniques may be used (e.g. Stemmer, Nature (1994) 370: 389-391). In further embodiments, one may generate novel H variable or L variable regions carrying one or more sequences derived from the sequences disclosed herein using random mutagenesis of one or more selected H variable and/or L variable genes, such as error-prone PCR (Proc. Nat. Acad. Sci. U.S.A. (1992) 89: 3576-3580). Another method that may be used is to direct mutagenesis to CDRs of H variable or L variable genes (Proc. Nat. Acad. Sci. U.S.A. (1994) 91: 3809-3813; J. Mol. Biol. (1996) 263: 551-567). Similarly, one or more, or all three CDRs may be grafted into a repertoire of H variable or L variable domains, which are then screened for an antigen-binding fragment specific for GPNMB.

A portion of an immunoglobulin variable domain may comprise at least one of the CDRs substantially as set out herein and, optionally, intervening framework regions as set out herein. The portion may include at least about 50% of either or both of FR1 and FR4, the 50% being the C-terminal 50% of FR1 and the N-terminal 50% of FR4. Additional residues at the N-terminal or C-terminal end of the substantial part of the variable domain may be those not normally associated with naturally occurring variable domain regions. For example, construction of antibodies by recombinant DNA techniques may result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below.

Although the embodiments illustrated, for example in PCT Publication No. WO2006/071441, comprise a “matching” pair of H variable and L variable domains, a skilled artisan will recognize that alternative embodiments may comprise antigen-binding fragments containing only a single CDR from either L variable or H variable domain. Either one of the single chain specific binding domains can be used to screen for complementary domains capable of forming a two-domain specific antigen-binding fragment capable of, for example, binding to GPNMB. The screening may be accomplished by phage display screening methods using the so-called hierarchical dual combinatorial approach disclosed in WO92/01047, in which an individual colony containing either an H or L chain clone is used to infect a complete library of clones encoding the other chain (L or H) and the resulting two-chain specific binding domain is selected in accordance with phage display techniques as described.

Anti-GPNMB antibodies described herein can be linked to another functional molecule, e.g., another peptide or protein (albumin, another antibody, etc.), toxin, radioisotope, cytotoxic or cytostatic agents. For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192; or 4,179,337. The antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

The disclosed antibodies may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain glycosylation site consensus sequences known in the art. Another means of increasing the number of carbohydrate moieties on the antibodies is by chemical or enzymatic coupling of glycosides to the amino acid residues of the antibody (WO87/05330; CRC Crit. Rev. Biochem., 22: 259-306, 1981). Removal of any carbohydrate moieties from the antibodies may be accomplished chemically or enzymatically (Arch. Biochem. Biophys., 259: 52, 1987; Anal. Biochem., 118: 131, 1981; Meth. Enzymol., 138: 350, 1987). The antibodies may also be tagged with a detectable, or functional, label. Detectable labels include radiolabels such as ¹³¹I or ⁹⁹Tc, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

The valency of the antibodies may be custom designed to affect affinity and avidity, retention time at binding sites (see e.g. Am H. Pathol, 2002 160:1597-1608; J. Med. Chem. 2002 45:2250-2259; Br. J. Cancer 2002 86:1401-1410; Biomol. Eng. 2001 18:95-108; Int J. Cancer 2002 100:367-374).

Multiple specificity (bifunctional) binding reagents may be designed based upon the GPNMB specific sequences of the invention (Biomol. Eng. 2001 18:31-40). For example, a bispecific or bifunctional antibody is an artificial hybrid antibody having two different heavy/light chain pairs and two different binding sites. Bispecific antibodies can be produced by a variety of methods including fusion of hybridomas or linking of Fab′ fragments (Clin. Exp. Immunol. 1990, 79: 315-321; J. Immunol. 199, 2148:1547-1553). Such bispecific antibodies can be generated comprising a specificity to GPNMB and a second specificity to a second molecule using techniques that are well known (Immunol Methods 1994, 4:72-81; Wright and Harris, supra.; Traunecker et al. 1992 Int. J. Cancer (Suppl.) 7:51-52). Bispecific antibodies prepared in this manner selectively kill cells expressing GPNMB.

Antibodies, for example in which CDR sequences differ only insubstantially from those set out in Tables 1-30, are encompassed within the scope of this invention. Typically, an amino acid is substituted by a related amino acid having similar charge, hydrophobic, or stereochemical characteristics. Such substitutions would be within the ordinary skills of an artisan. Unlike in CDRs, more substantial changes can be made in FRs without adversely affecting the binding properties of an antibody. Changes to FRs include, but are not limited to engineering certain framework residues that are important for antigen contact or for stabilizing the binding site, e.g., changing the class or subclass of the constant region, changing specific amino acid residues which might alter the effector function such as Fc receptor binding (U.S. Pat. Nos. 5,624,821; 5,648,260; Lund et al. (1991) J. Immun. 147: 2657-2662; Morgan et al. (1995) Immunology 86: 319-324), or changing the species from which the constant region is derived.

One of skill in the art will appreciate that the derivatives and modifications described above are not all-exhaustive, and that many other modifications would be obvious to a skilled artisan in light of the teachings of the present disclosure.

### Immunoconjugates

In another aspect, the antibodies of the invention and derivatives thereof can be used as a targeting agent for delivery of another therapeutic or a cytotoxic agent to a cell expressing GPNMB. The method includes administering an anti-GPNMB antibody coupled to a therapeutic or a cytotoxic agent or under conditions that allow binding of the antibody to GPNMB.

Anti-GPNMB antibodies are conjugated to a therapeutic agent, such as a cytotoxic compound, such that the resulting immunoconjugate exerts a cytotoxic or cytostatic effect on a GPNMB expressing cell. Particularly suitable moieties for conjugation to antibodies are chemotherapeutic agents, prodrug converting enzymes or toxins. For example, an anti-GPNMB antibody can be conjugated to a cytotoxic agent such as a chemotherapeutic agent (see infra) or a toxin (e.g. abrin, ricin A, pseudomonas exotoxin, or diphtheria toxin). Alternatively, anti-GPNMB antibody may be conjugated to a pro-drug converting enzyme. The pro-drug converting enzyme can be recombinantly fused to the antibody or derivative thereof or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Any agent that exerts a therapeutic effect on GPNMB expressing cells can be used as an agent for conjugation to an anti-GPNMB antibody of the invention. Useful classes of cytotoxic agents include, for example, antitubulin agents, auristatins, DNA minor groove binders, NDA replication inhibitors, alkylating agents (e.g., platinum complexes such as cis-plantin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated purimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like.

The therapeutic agent can be a cytotoxic agent. Suitable cytotoxic agents include, for example, dolastatins (e.g. auristatin E, AFP, MMAF, MMAE), DNA minor groove binders (e.g., enediynes and lexitropsins), cuocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

In a specific embodiment, the cytotoxic or cytostatic agent is auristatin E (dolastatin-10) or a derivative thereof (e.g. an ester formed between auristatin E and a keto acid). Other typical auristatin derivatives include AFP, MMAR, and MMAE. The synthesis and structure of auristatin E and its derivates are described in U.S. Patent Application Publication No. 20030083263; PCT/US03/24209; PCT/US02/13435; and U.S. Pat. Nos. 6,323,315; 6,239,104; 6,034065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

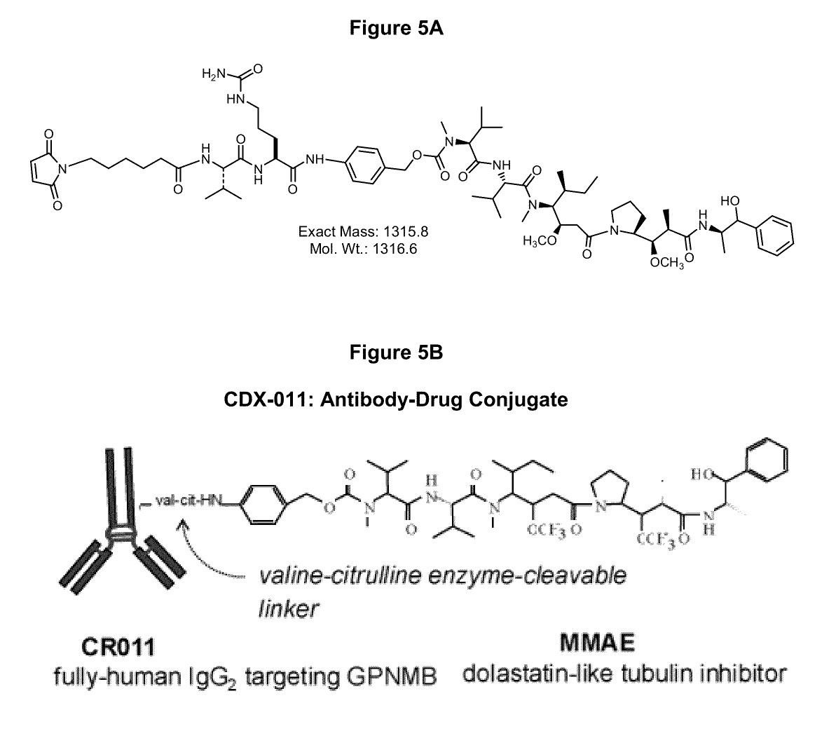

In a specific embodiment anti-GPNMB antibody 1.15.1 was coupled to monomethylauristatin E via intracellular protease-sensitive valine-citrulline peptide linker (vcMMAE). This antibody conjugate is also referred to herein as CR011vcMMAE and CDX-011 and glembatumumab vedotin. Methods for making the immunoconjugate can be found in Doronina S. O. et al, 2003 Nature Biotechnology 21(7):778-794. The structure of vcMMAE is shown in FIG. 5A.

Techniques for conjugating therapeutic agents to proteins, and in particular, antibodies are known in the art (see, e.g. Amon et al., 1985 in Monoclonal Antibodies and Cancer Therapy, Reisfeld et al. eds., Alan R. Liss, Inc., 1985; Hellstrom et al., 1987 in Controlled Drug Delivery, Robinson et al. eds., Marcel Dekker, Inc., 2^{nd} ed. 1987; Thorpe 1985, in Monoclonal Antibodies '84: Biological and Clinical Applications, Pinchera et al. eds., EDITOR, 1985; Monoclonal Antibodies for Cancer Detection and Therapy, Baldwin et al. eds., Academic Press 1985; and Thorpe et al., 1982, Immunol. Rev. 62:119-58).

In certain embodiments of the invention, anti-GPNMB antibodies binding to GPNMB expressing cells, are internalized and accumulate in the cell. Thereby anti-GPNMB antibody immunoconjugates accumulate in GPNMB expressing cells. Typically when the anti-GPNMB antibody immunoconjugate is internalized, the agent is preferentially active. Alternatively, anti GPNMB immunoconjugates are not internalized and the drug is effective to deplete or inhibit GPNMB expressing cells by binding to the cell membrane. The therapeutic agent can be conjugated in a manner that reduces its activity unless it is cleaved off the antibody (e.g. by hydrolysis or by a cleaving agent). In this case, the agent can be attached to the antibody or derivative thereof with a cleavable linker that is sensitive to cleavage in the intracellular environment of the target but is not substantially sensitive to the extracellular environment, such that the conjugate is cleaved from the antibody or derivative thereof when it is internalized by the GPNMB expressing cell (e.g. in the endosomal or, for example by virtue of pH sensitivity or protease sensitivity, in the lysosomal environment or in a caveolea).

A therapeutic agent of the immunoconjugate can be charged relative to the plasma membrane (e.g. polarized or net charge relative to the plasma membrane), thereby further minimizing the ability of the agent to cross the plasma membrane once internalized by a cell.

The anti-GPNMB antibody immunoconjugate can comprise a linker region between the therapeutic agent and the antibody. The linker can be cleavable under intracellular conditions, such that cleavage of the linker releases the therapeutic agent from the antibody in the intracellular environment. The linker can be, e.g. a peptidyl linker that is cleaved by an intracellular peptidase or protease enzyme, including but not limited to a lysosomal or endosomal protease. Often the peptidyl linker is at least two amino acids long or at least three amino acids long. Cleaving agents can include cathepsins and D and plasmin, all of which are known to hydrolyze dipeptide drug derivative s resulting in the release of active drug inside target cells (see Dubowchik and Walker, 1999 Pharm. Therapeutics 83:67-123). Other linkers are described e.g. in U.S. Pat. No. 6,214,345.

Linkers can be pH-sensitive can often be hydrolizable under acidic conditions such as is found in the lysosome (see e.g. U.S. Pat. Nos. 5,122,368; 5,824,805; 5,622,929; Dubowchik and Walker, 1999 Pharm. Therapeutics 83:67-123; Neville et al., 1989 Biol. Chem. 264:14653-14661). Such linkers are relatively stable under neutral pH conditions, such as those in the blood, but are unstable at below pH 5.5 or 5.0, the pH of the lysosome. Linkers can be cleavable under reducing conditions (e.g. a disulfide linker) (see e.g., Thorpe et al., 1987 Cancer Res. 47:5924-5931; Wawrzynczak et al., In Immunoconjugates: Antibody Conjugates in Radioimagery and Therapy of Cancer, C. W. Vogel ed., Oxford U. Press, 1987; U.S. Pat. No. 4,880,935). The linker can be a malonate linker (Johnson et al., 1995, Anticancer Res. 15:1387-93), a maleimidobenzoyl linker (Lau et al., 1995, Bioorg-Med-Chem. 3(10):1299-1304) or a 3′-N-amide analog (Lau et al., 1995, Bioorg-Med-Chem. vol. 3(10):1305-1312).

### Anti-GPNMB Therapies in Breast Cancer

The anti-GPNMB antibodies, immunoconjugates and other derivatives provided herein are useful in treating, ameliorating, delaying the onset and/or progression or otherwise reducing the severity of a breast cancer or one or more symptoms thereof. Efficacy of treatment is determined in association with any known method for diagnosing or treating the particular inflammatory-related disorder. Alleviation of one or more symptoms of the inflammatory-related disorder indicates that the antibody confers a clinical benefit.

It is now commonly understood that breast cancer is not one form of cancer, but many different “subtypes” of cancer, These subtypes of breast cancer are generally diagnosed based upon the presence, or lack of, three receptors known to fuel most breast cancers: estrogen receptors (ER), progesterone receptors (PR) and human epidermal growth factor receptor 2 (HER2). Most current treatments for breast cancer target these receptors. Some subjects have a form of breast cancer known as “triple negative breast cancer” in which none of these receptors are found, i.e., a triple negative breast cancer diagnosis refers to breast cancer tumors that are estrogen receptor-negative, progesterone receptor-negative and HER2-negative. Because of the triple negative status, triple negative breast cancer tumors generally do not respond to receptor targeted treatments. Depending on the stage of its diagnosis, triple negative breast cancer can be particularly aggressive, and more likely to recur than other subtypes of breast cancer. Accordingly, there exists a need in the art for new therapies that target triple negative breast cancers.

The molecular classification of breast cancer underscores the heterogeneity of this disease (Perou et al., Nature 2000; 406:747-52; Sorlie et al., Proc Natl Acad Sci USA 2001; 98:10869-74; and Sorlie T et al., Proc Natl Acad Sci USA 2003; 100:8418-23). The poor prognosis associated with triple-negative breast cancer, coupled with the lack of therapeutic targets, has created intense clinical interest in these tumors (Reis-Filho J S, Tutt A N. Triple negative tumours: a critical review. Histopathology 2008; 52:108-18; Nofech-Mozes S, Trudeau M, Kahn H K, et al. Patterns of recurrence in the basal and non-basal subtypes of triple-negative breast cancers. Breast Cancer Res Treat 2009; 118:131-7; Mullan P B, Millikan R C. Molecular sub-typing of breast cancer: opportunities for new therapeutic approaches. Cell Mol Life Sci 2007; 64:3219-321; and Rakha E A, Reis-Filho J S, Ellis I O. Basal-like breast cancer: a critical review. J Clin Oncol 2008; 26:2568-81). Recent studies have reported that triple-negative tumors with basal-like features (those expressing some or all of the following proteins: CK5/6, CK14, CK17, epidermal growth factor receptor) are associated with worse clinical outcomes than triple-negative tumors lacking these markers (Nofech-Mozes et al., Breast Cancer Res Treat 2009; 118:131-7; and Rakha E A, Elsheikh S E, Aleskandarany M A, et al. Triple-negative breast cancer: distinguishing between basal and nonbasal subtypes. Clin Cancer Res 2009; 15:2302-10). Although it is unknown whether GPNMB contributes to the basal-like phenotype, the observations provided herein identify GPNMB as a prognostic marker in triple-negative breast cancers and support the clinical development of GPNMB-targeted therapies. Recent evidence suggests that signaling through the estrogen receptor can suppress GPNMB expression (Stender J D, Frasor J, Komm B, Chang K C, Kraus W L, Katzenellenbogen B S. Estrogen-regulated gene networks in human breast cancer cells: involvement of E2F1 in the regulation of cell proliferation. Mol Endocrinol 2007; 21:2112-23; and Yau C, Benz C C. Genes responsive to both oxidant stress and loss of estrogen receptor function identify a poor prognosis group of estrogen receptor positive primary breast cancers. Breast Cancer Res 2008; 10:R61), which is consistent with the observation herein that GPNMB is more commonly expressed in triple-negative breast cancers.

Gene expression profiling studies have revealed that higher GPNMB levels in tumor-associated stroma compared with that derived from normal breast (Finak G, Bertos N, Pepin F, et al. Stromal gene expression predicts clinical outcome in breast cancer. Nat Med 2008; 14:518-27; and Karnoub A E, Dash A B, Vo A P, et al. Mesenchymal stem cells within tumour stroma promote breast cancer metastasis. Nature 2007; 449:557-63). Within the stromal compartment, an independent study identified increased GPNMB expression in tumor-derived endothelium relative to normal endothelial cells (Ghilardi C, Chiorino G, Dossi R, Nagy Z, Giavazzi R, Bani M. Identification of novel vascular markers through gene expression profiling of tumor-derived endothelium. BMC Genomics 2008; 9:201). GPNMB is highly expressed in dendritic cells (Shikano S, Bonkobara M, Zukas P K, Ariizumi K. Molecular cloning of a dendritic cell-associated transmembrane protein, DC-HIL, that promotes RGD-dependent adhesion of endothelial cells through recognition of heparan sulfate proteoglycans. J Biol Chem 2001; 276:8125-34) and macrophages (Ripoll et al., J Immunol 2007; 178:6557-66), raising the possibility that some of the stromal staining within primary breast tumors may represent immune cell infiltrates. Moreover, osteoactivin expression has been linked to fibroblast activation (Ogawa T, Nikawa T, Furochi H, et al. Osteoactivin upregulates expression of MMP-3 and MMP-9 in fibroblasts infiltrated into denervated skeletal muscle in mice. Am J Physiol Cell Physiol 2005; 289: C697-707) and, thus, is likely to be expressed in cancer-associated fibroblasts.

These studies are the first to show that GPNMB-expressing breast cancer cells can be selectively killed by a toxin-conjugated antibody directed against GPNMB (CDX-011). Cancer therapy using toxin/drug-conjugated antibodies is becoming increasingly popular (Carter P J, Senter P D. Antibody-drug conjugates for cancer therapy. Cancer J 2008; 14:154-69) and includes a cytotoxin-conjugated version of Herceptin, Trastuzumab-DM1, which is currently being investigated in clinical trials for metastatic breast cancer (Carter et al., Cancer J 2008; 14:154-69; and Lewis Phillips G D, Li G, Dugger D L, et al. Targeting HER2-positive breast cancer with trastuzumab-DM1, an antibody-cytotoxic drug conjugate. Cancer Res 2008; 68:9280-90). In a phase I/II clinical trial for the treatment of melanoma, CDX-011 was shown to have clinical activity and was well tolerated (Carter et al., Cancer J 2008; 14:154-69).

Moreover, initial results from a phase I/II trial show that tumor shrinkage was observed in CDX-011-treated patients with metastatic breast cancer. The observations provided herein that GPNMB is highly expressed in recurrent breast cancers but rarely in normal breast tissue, coupled with the observations that CDX-011 effectively inhibits the growth of GPNMB-expressing breast cancer cells in vitro, indicate that the methods of the invention, which target GPNMB, are useful in treating breast cancer. Immunohistochemistry staining of biopsy material for epithelial GPNMB expression is useful to predict responders to anti-GPNMB treatment. The molecular processes that modulate cell surface expression of GPNMB, such as trafficking, internalization, and shedding of its extracellular domain (Qian X, Mills E, Torgov M, Larochelle W J, Jeffers M. Pharmacologically enhanced expression of GPNMB increases the sensitivity of melanoma cells to the CR011-vcMMAE antibody-drug conjugate. Mol Oncol 2008; 2:81-93; and Furochi H, Tamura S, Mameoka M, et al. Osteoactivin fragments produced by ectodomain shedding induce MMP-3 expression via ERK pathway in mouse NIH-3T3 fibroblasts. FEBS Lett 2007; 581:5743-50) are characterized to optimize GPNMB-targeted therapies.

The present invention provides methods for treating and/or preventing a disease or disorder associated with overexpression of GPNMB and/or cell hyperproliferative disorders, particularly cancer and more particularly a breast cancer, in a subject comprising administering an effective amount of an antibody that targets cells expressing GPNMB, and inhibiting or otherwise modulating GPNMB expression or function. In one embodiment, the method of the invention comprises administering to a subject a composition comprising an immunoconjugate that comprises an antibody of the invention and a cytotoxic agent against the hyperproliferative cell disease. The antibodies can be used to prevent, diagnose, or treat breast cancer in a subject, especially in humans. Antibodies of the invention can also be used for isolating GPNMB or GPNMB-expressing cells, e.g., from breast cancer tumors. Furthermore, the antibodies can be used to treat a subject at risk of or susceptible to a breast cancer, or to treat a subject currently suffering from a breast cancer.

The present invention provides therapies comprising administering one of more antibodies of the invention and compositions comprising said antibodies to a subject, preferably a human subject, for preventing and/or treating a breast cancer or a symptom thereof. In one embodiment, the method includes administering to a subject in need thereof an effective amount of one or more antibodies of the invention or an immunoconjugate or other derivative or antigen-binding fragment thereof. In certain embodiments, an effective amount of one or more immunoconjugates comprising one or more antibodies of the invention is administered to a subject in need thereof to prevent or treat a breast cancer or a symptom thereof.

The invention also provides methods of preventing or treating a breast cancer by administering to a subject in need thereof one or more of the antibodies of the invention and one or more therapies (e.g., one or more prophylactic or therapeutic agents) other than antibodies of the invention. The therapeutic compositions of the invention, which include one or more of the anti-GPNMB antibodies, conjugates and other derivatives thereof described herein, are useful in conjunction with any of a variety of known treatments for locally advanced and/or metastatic including, by way of non-limiting example, surgical treatments and methods, radiation therapy, chemotherapy and/or hormone or other endocrine-related treatment. The prophylactic or therapeutic agents of the combination therapies of the invention can be administered sequentially or concurrently. In a specific embodiment, the combination therapies of the invention comprise an effective amount of one or more antibodies of the invention and an effective amount of at least one other therapy (e.g., prophylactic or therapeutic agent) which has a different mechanism of action than the antibodies. In certain embodiments, the combination therapies of the present invention improve the prophylactic or therapeutic effect of one or more antibodies of the invention by functioning together with the antibodies to have an additive or synergistic effect. In certain embodiments, the combination therapies of the present invention reduce the side effects associated with the therapies (e.g., prophylactic or therapeutic agents).

The prophylactic or therapeutic agents of the combination therapies can be administered to a subject, preferably a human subject, in the same pharmaceutical composition. Alternatively, the prophylactic or therapeutic agents of the combination therapies can be administered concurrently, separately or sequentially to a subject in separate pharmaceutical compositions. The prophylactic or therapeutic agents may be administered to a subject by the same or different routes of administration. Such agents may include for example chemotherapy, taxane, capecitabine, anthracycline, hormonal therapy, gemcitabine, vinorelbine, epothilone, lapatinib or antibody therapies such as bevacizumab or trastuzumab.

The anti-GPNMB antibodies, conjugates and other derivatives thereof are used to treat, delay the progression of, alleviate a symptom of, or otherwise ameliorate a locally advanced and/or metastatic breast cancer in a subject. Symptoms associated with locally advanced and/or metastatic breast cancer include, for example, a tumor greater than 5 cm across, a fixed lump in the axilla (i.e., underarm), ulceration of the skin, involvement of the deep chest muscles, involvement of multiple lymph nodes in the local area including, e.g., those located in the axilla and/or in the soft tissues above or below the collarbone.

The therapeutic compositions of the invention, which include one or more of the anti-GPNMB antibodies, conjugates and other derivatives thereof described herein, are administered to a subject suffering from a breast cancer, such as for example, a basal-like breast cancer, a triple negative breast cancer, a locally advanced breast cancer and/or a metastatic breast cancer. A subject suffering from a breast cancer is identified by methods known in the art. For example, subjects suffering from a breast cancer are identified using any of a variety of clinical and/or laboratory tests such as, physical examination, biopsy, radiologic examination and blood, urine and stool analysis to evaluate immune status.

Administration of the therapeutic compositions of the invention, which include one or more of the anti-GPNMB antibodies, conjugates and other derivatives thereof described herein, to a patient suffering from a breast cancer may be considered successful if any of a variety of laboratory or clinical results is achieved. For example, administration of the therapeutic compositions of the invention, which include one or more of the anti-GPNMB antibodies, conjugates and other derivatives thereof described herein, to a patient suffering from a breast cancer such as, for example, a basal-like breast cancer, a triple negative breast cancer, a locally advanced breast cancer and/or a metastatic breast cancer, is considered successful one or more of the symptoms associated with the breast cancer is alleviated, reduced, inhibited or does not progress to a further, i.e., worse, state. Administration of the therapeutic compositions of the invention, which include one or more of the anti-GPNMB antibodies, conjugates and other derivatives thereof described herein, to a patient suffering from a breast cancer such as, for example, a basal-like breast cancer, a triple negative breast cancer, a locally advanced breast cancer and/or a metastatic breast cancer, may be considered successful if the breast cancer enters remission or does not progress to a further, i.e., worse, state.

### Dosage and Frequency of Administration

The amount of a prophylactic or therapeutic agent or a composition of the invention which will be effective in the prevention and/or treatment of a disorder associated with or characterized by aberrant expression and/or activity of GPNMB can be determined by standard clinical methods. For example, the dosage of the composition which will be effective in the treatment and/or prevention of cancer can be determined by administering the composition to an animal model. In addition, in vitro assays may optionally be employed to help identify optimal dosage ranges. Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals. The data obtained from the cell culture assays or animal studies can be used in formulating a range of dosage for use in humans. Therapeutically effective dosages achieved in one animal model can be converted for use in another animal, including humans, using conversion factors known in the art (see, e.g., Freireich et al. (1966) Cancer Chemother. Reports, 50(4): 219-244).

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, gender, immune status and other factors known by the skilled artisan to reflect the accuracy of administered pharmaceutical compositions. Suitable regimens can be selected by one skilled in the art by considering such factors and by following, for example, dosages reported in literature and recommended in the Physician's Desk Reference (59th ed., 2005).

The precise dose to be employed in the formulation will also depend on the route of administration, and the seriousness of the cancer, and should be decided according to the judgment of the practitioner and each patient's circumstances. Effective doses may be extrapolated from dose-response curves derived from in vitro or animal model test systems.

For other cancer therapeutic agents administered to a patient, the typical doses of various cancer therapeutics are known in the art. Given the invention, certain preferred embodiments will encompass the administration of lower dosages in combination treatment regimens than dosages recommended for the administration of single agents.

In a specific embodiment, the dosage of an antibody or an immunoconjugate comprising an antibody of the invention administered to prevent and/or treat a disorder associated with or characterized by aberrant expression and/or activity of GPNMB (e.g., cancer) in a patient is 30 mg/kg or less, 25 mg/kg or less, 20 mg/kg or less, 15 mg/kg or less, preferably 12 mg/kg or less, 11 mg/kg or less, 10 mg/kg or less, 9 mg/kg or less, 8 mg/kg or less, 7 mg/kg or less, 6 mg/kg or less, 5 mg/kg or less, 4 mg/kg or less, 3 mg/kg or less, 2 mg/kg or less, or 1 mg/kg or less of a patient's body weight. In another embodiment, the dosage of an antibody or an immunoconjugate of the invention administered to prevent and/or treat a disorder associated with or characterized by aberrant expression and/or activity of GPNMB (e.g., cancer) in a patient is a unit dose of about 0.01 mg/kg to about 20 mg/kg, about 0.1 mg/kg to about 10 mg/kg, about 0.1 mg/kg to about 8 mg/kg, about 0.1 mg/kg to about 7 mg/kg, about 0.1 mg/kg to about 6 mg/kg, about 0.1 mg/kg to about 5 mg/kg, about 0.1 mg/kg to about 4 mg/kg, preferably, about 0.1 mg/kg to about 3 mg/kg, about 0.2 mg/kg to 3 mg/kg, about 0.3 mg/kg to about 3 mg/kg, about 0.4 mg/kg to about 3 mg/kg, about 0.6 mg/kg to about 3 mg/kg, about 0.8 mg/kg to about 3 mg/kg, about 0.1 mg/kg to 2 mg/kg, about 0.1 mg/kg to 1 mg/kg. In certain embodiments, the dosage of an antibody or an immunoconjugate comprising an antibody of the invention administered to prevent and/or treat a disorder associated with or characterized by aberrant expression and/or activity of GPNMB (e.g., cancer) in a patient is a unit dose of about 0.1 mg/kg, about 0.2 mg/kg, about 0.4 mg/kg, about 0.6 mg/kg, about 0.8 mg/kg, about 1.1 mg/kg, or about 1 mg/kg.

In certain embodiments, a subject is administered one or more doses of an effective amount of one or more antibodies or immunoconjugates of the invention to prevent and/or treat a disorder associated with or characterized by aberrant expression and/or activity of GPNMB, wherein the dose of an effective amount of said antibodies, immunoconjugates, compositions, or combination therapies reduces and/or inhibits proliferation of cancerous cells by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art.

In other embodiments, a subject is administered one or more doses of an effective amount of one or more antibodies or immunoconjugates of the invention to prevent and/or treat a disorder associated with or characterized by aberrant expression and/or activity of GPNMB, wherein the dose of an effective amount achieves a serum titer of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the antibodies of the invention. In yet other embodiments, a subject is administered a dose of an effective amount of one or more antibodies or immunoconjugates of the invention to achieve a serum titer of at least 0.1 μg/mL, at least 0.5 μg/mL, at least 1 μg/mL, at least, 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the antibodies and a subsequent dose of an effective amount of one or more antibodies or immunoconjugates of the invention is administered to maintain a serum titer of at least 0.1 μg/mL, at least 0.5 μg/mL, at least 1 μg/mL, at least, 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL. In accordance with these embodiments, a subject may be administered 1, 2, 3, 4, 5, 6, 7, 8, 9, 10, 11, 12 or more subsequent doses.

In a specific embodiment, the invention provides methods of preventing and/or treating a disorder associated with or characterized by aberrant expression and/or activity of GPNMB, said method comprising administering to a subject in need thereof a unit dose of at least 0.01 mg/kg, at least 0.1 mg/kg, at least 0.2 mg/kg, at least 0.4 mg/kg, at least 0.6 mg/kg, at least 0.8 mg/kg, at least 1 mg/kg, or at least 1.1 mg/kg of one or more antibodies or immunoconjugates of the invention. In another embodiment, the invention provides methods of preventing and/or treating a disorder associated with or characterized by aberrant expression and/or activity of GPNMB, said method comprising administering to a subject in need thereof a unit dose of at least 0.01 mg/kg, at least 0.1 mg/kg, at least 0.2 mg/kg, at least 0.4 mg/kg, at least 0.6 mg/kg, at least 0.8 mg/kg, at least 1 mg/kg, or at least 1.1 mg/kg of one or more antibodies or immunoconjugates of the invention once every 7 days, preferably, once every 10 days, once every 12 days, once every 14 days, once every 16 days, once every 18 days, once every three weeks (21 days), or once a month. In a preferred embodiment, an immunoconjugate of the instant invention is administered intravenously at a unit dose of about 0.1 mg/kg, about 0.2 mg/kg, about 0.4 mg/kg, about 0.6 mg/kg, about 0.8 mg/kg, about 1.1 mg/kg, or about 1 mg/kg once every 10 to 30 days, for example once every 21 days with 2 to 4 or more cycles.

The present invention provides methods of preventing and/or treating a disorder associated with or characterized by aberrant expression and/or activity of GPNMB, said method comprising: (a) administering to a subject in need thereof one or more doses of a prophylactically or therapeutically effective amount of one or more antibodies or immunoconjugates of the invention; and (b) monitoring the plasma level/concentration of the said administered antibody or antibodies in said subject after administration of a certain number of doses of the said antibody or antibodies. Moreover, preferably, said certain number of doses is 1, 2, 3, 4, 5, 6, 7, or 8 doses of a prophylactically or therapeutically effective amount one or more antibodies or immunoconjugates of the invention.

In a specific embodiment, the invention provides a method of preventing and/or treating a disorder associated with or characterized by aberrant expression and/or activity of GPNMB, said method comprising: (a) administering to a subject in need thereof a dose of at least 0.1 mg/kg (preferably at least at least 0.2 mg/kg, at least 0.4 mg/kg, at least 0.6 mg/kg, at least 0.8 mg/kg, at least 1 mg/kg, or at least 1.1 mg/kg) of one or more antibodies or immunoconjugates of the invention; and (b) administering one or more subsequent doses to said subject when the plasma level of the antibody or antibodies administered in said subject is less than 0.1 μg/mL, preferably less than 0.25 μg/mL, less than 0.5 μg/mL, less than 0.75 μg/mL, or less than 1 μg/mL. In another embodiment, the invention provides a method of preventing and/or treating a disorder associated with or characterized by aberrant expression and/or activity of GPNMB, said method comprising: (a) administering to a subject in need thereof one or more doses of at least at least 0.1 mg/kg (preferably at least at least 0.2 mg/kg, at least 0.4 mg/kg, at least 0.6 mg/kg, at least 0.8 mg/kg, at least 1 mg/kg, or at least 1.1 mg/kg) of one or more antibodies of the invention; (b) monitoring the plasma level of the administered antibody or antibodies of the invention in said subject after the administration of a certain number of doses; and (c) administering a subsequent dose of the antibody or antibodies of the invention when the plasma level of the administered antibody or antibodies in said subject is less than 0.1 μg/mL, preferably less than 0.25 μg/mL, less than 0.5 μg/mL, less than 0.75 μg/mL, or less than 1 μg/mL. Preferably, said certain number of doses is 1, 2, 3, 4, 5, 6, 7, or 8 doses of an effective amount of one or more antibodies or immunoconjugates of the invention.

Therapies (e.g., prophylactic or therapeutic agents), other than antibodies or immunoconjugates of the invention, which have been or are currently being used to prevent and/or treat a disorder associated with or characterized by aberrant expression and/or activity of GPNMB can be administered in combination with one or more antibodies or immunoconjugates of the invention according to the methods of the invention to treat and/or prevent a disorder associated with or characterized by aberrant expression and/or activity of GPNMB. Preferably, the dosages of prophylactic or therapeutic agents used in combination therapies of the invention are lower than those which have been or are currently being used to prevent and/or treat a disorder associated with or characterized by aberrant expression and/or activity of GPNMB.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more antibodies of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

### Pharmaceutical Compositions and Methods of Administration

The disclosure provides compositions comprising anti-GPNMB antibodies. Such compositions may be suitable for pharmaceutical use and administration to patients. The compositions typically comprise one or more antibodies of the present invention and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars; polyalcohols such as mannitol, sorbitol, and sodium chloride in the composition. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

Oral compositions generally include an inert diluent or an edible carrier. They can be enclosed in gelatin capsules or compressed into tablets. For oral administration, the antibodies can be combined with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers, or suppositories. For example, in case of antibodies that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the antibodies may be delivered in the form of an aerosol spray from pressured container or dispenser, which contains a suitable propellant, e.g., a gas such as carbon dioxide, or a nebulizer.

In certain embodiments, the presently disclosed antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

For any composition used in the present invention, the therapeutically effective dose can be estimated initially from cell culture assays. Examples of suitable bioassays include DNA replication assays, clonogenic assays and other assays as, for example, described in the Examples. The data obtained from the cell culture assays and animal studies can be used in formulating a range of dosage for use in humans. A dose may be formulated in animal models to achieve a circulating plasma concentration range that includes the IC₅₀ (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

Antibodies can be modified to become immunotoxins utilizing techniques that are well known in the art (Vitetta 1993, Immunol Today 14:252; U.S. Pat. No. 5,194,594). Cytotoxic immunoconjugates are known in the art and have been used as therapeutic agents. Such immunoconjugates may for example, use maytansinoids (U.S. Pat. No. 6,441,163), tubulin polymerization inhibitor, auristatin (Mohammad et al, 1999 Int. J. Oncol 15(2):367-72; Doronina et al, 2003 Nature Biotechnology 21(7):778-784), dolastatin derivatives (Ogawa et al, 2001 Toxicol Lett. 121(2):97-106) 21(3)778-784), Mylotarg® (Wyeth Laboratories, Philadelphia, Pa.); maytansinoids (DM1), taxane or mertansine (ImmunoGen Inc.).

Immunoradiopharmaceuticals utilizing anti-GPNMB antibodies may be prepared utilizing techniques that are well known in the art (Junghans et al. in Cancer Chemotherapy and Biotherapy 655-686 (2d edition, Chafner and Longo, eds., Lippincott Raven (1996); U.S. Pat. Nos. 4,681,581, 4,735,210, 5,101,827, 5,102,990 (RE 35,500), 5,648,471, and 5,697,902). Each of the immunotoxins and radiolabeled antibody molecules selectively kill cells expressing GPNMB. Radiolabels are known in the art and have been used for diagnostic or therapeutic radioimmuno conjugates. Examples of radiolabels include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I, ¹⁷⁷Lu, ¹⁰⁵, Rh, Rhenium-186, Rhenium-188, Samarium-153, Copper-64, and Scandium-47). For example, radionuclides which have been used in radioimmunoconjugate guided clinical diagnosis include, but are not limited to: ¹³¹I, ¹²⁵I, ¹²³I, ⁹⁹Tc, ⁶⁷Ga, as well as ¹¹¹In. Antibodies have also been labeled with a variety of radionuclides for potential use in targeted immunotherapy (see Peirersz et al., 1987). These radionuclides include, for example, ¹⁸⁸Re and ¹⁸⁶Re as well as ⁹⁰Y, and to a lesser extent ¹⁹⁹Au and ⁶⁷Cu. I-(131) (see for example U.S. Pat. No. 5,460,785). Radiotherapeutic chelators and chelator conjugates are known in the art (U.S. Pat. Nos. 4,831,175, 5,099,069, 5,246,692, 5,286,850, and 5,124,471).

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, human, humanized, single-chain, chimeric, synthetic, recombinant, hybrid, mutated, engineered, and grafted antibodies. Unless otherwise modified by the term “intact,” as in “intact antibodies,” for the purposes of this disclosure, the term “antibody” also includes antibody fragments such as Fab, F(ab′)₂, Fv, scFv, bi-scFv, bi-Ab, Fd, dAb, and other antibody fragments that retain antigen-binding function, i.e., the ability to bind GPNMB specifically. Typically, such fragments would comprise an antigen-binding domain. An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

As used herein, the terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” By “specifically bind” or “immunoreacts with” or “directed against” is meant that the antibody reacts with one or more antigenic determinants of the desired antigen and does not react with other polypeptides or binds at much lower affinity (K_{d}>10⁻⁶). \

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is typically formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an intact antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM; e.g., ≦100 nM, preferably ≦10 nM and more preferably ≦1 nM.

As used herein, the terms “immunological binding,” and “immunological binding properties” typically refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is said to specifically bind to GPNMB, when the equilibrium binding constant (K_{d}) is ≦1 μM, preferably ≦100 nM, more preferably ≦10 nM, and most preferably ≦100 pM to about 1 pM, as measured by assays such as radioligand binding assays or similar assays known to those skilled in the art.

As used herein, the term “substantially as set out” refers that the relevant CDR, V_{H}, or V_{L} domain of the invention will be either identical to or have only insubstantial differences in the specified regions (e.g., a CDR), the sequence of which is set out. Insubstantial differences include minor amino acid changes, such as substitutions of 1 or 2 out of any 5 amino acids in the sequence of a specified region.

As used herein, the terms “CR011” and variants thereof refer to a fully human monoclonal antibody that specifically binds to GPNMB. This antibody is also referred to herein as Mab 1.15.1 as described in the instant invention. The terms “CDX-011” and CR011vcMMAE refer to the antibody-drug conjugate comprising the CR011 antibody coupled to monomethylauristatin E via intracellular protease-sensitive valine-citrulline peptide linker. The structure of MMAE is shown in FIG. 5A, and the structure of the CDX-011 antibody-drug conjugate is shown in FIG. 5B.

The terms “GPNMB” and “CG56972” are used interchangeably herein. As used herein, the terms “GPNMB” or “CG56972” refer to a transmembrane glycoprotein that has an amino acid sequence as set forth below in SEQ ID NO: 271. GPNMB proteins and polypeptides include the mature, processed form of GPNMB, the extracellular domain of GPNMB, analogs, derivatives or fragments of the amino acid sequence as set forth in SEQ ID NO: 271. GPNMB amino acid sequence:

(SEQ ID NO: 271)
Lys Arg Phe His Asp Val Leu Gly Asn Glu Arg Pro Ser Ala Tyr Met Arg Glu His Asn Gln
Leu Asn Gly Trp Ser Ser Asp Glu Asn Asp Trp Asn Glu Lys Leu Tyr Pro Val Trp Lys Arg
Gly Asp Met Arg Trp Lys Asn Ser Trp Lys Gly Gly Arg Val Gln Ala Val Leu Thr Ser Asp
Ser Pro Ala Leu Val Gly Ser Asn Ile Thr Phe Ala Val Asn Leu Ile Phe Pro Arg Cys Gln
Lys Glu Asp Ala Asn Gly Asn Ile Val Tyr Glu Lys Asn Cys Arg Asn Glu Ala Gly Leu Ser
Ala Asp Pro Tyr Val Tyr Asn Trp Thr Ala Trp Ser Glu Asp Ser Asp Gly Glu Asn Gly Thr
Gly Gln Ser His His Asn Val Phe Pro Asp Gly Lys Pro Phe Pro His His Pro Gly Trp Arg
Arg Trp Asn Phe Ile Tyr Val Phe His Thr Leu Gly Gln Tyr Phe Gln Lys Leu Gly Arg Cys
Ser Val Arg Val Ser Val Asn Thr Ala Asn Val Thr Leu Gly Pro Gln Leu Met Glu Val Thr
Val Tyr Arg Arg His Gly Arg Ala Tyr Val Pro Ile Ala Gln Val Lys Asp Val Tyr Val Val
Thr Asp Gln Ile Pro Val Phe Val Thr Met Phe Gln Lys Asn Asp Arg Asn Ser Ser Asp Glu
Thr Phe Leu Lys Asp Leu Pro Ile Met Phe Asp Val Leu Ile His Asp Pro Ser His Phe Leu
Asn Tyr Ser Thr Ile Asn Tyr Lys Trp Ser Phe Gly Asp Asn Thr Gly Leu Phe Val Ser Thr
Asn His Thr Val Asn His Thr Tyr Val Leu Asn Gly Thr Phe Ser Leu Asn Leu Thr Val Lys
Ala Ala Ala Pro Gly Pro Cys Pro Pro Pro Pro Pro Pro Pro Arg Pro Ser Lys Pro Thr Pro
Ser Leu Ala Thr Thr Leu Lys Ser Tyr Asp Ser Asn Thr Pro Gly Pro Ala Gly Asp Asn Pro
Leu Glu Leu Ser Arg Ile Pro Asp Glu Asn Cys Gln Ile Asn Arg Tyr Gly His Phe Gln Ala
Thr Ile Thr Ile Val Glu Gly Ile Leu Glu Val Asn Ile Ile Gln Met Thr Asp Val Leu Met
Pro Val Pro Trp Pro Glu Ser Ser Leu Ile Asp Phe Val Val Thr Cys Gln Gly Ser Ile Pro
Thr Glu Val Cys Thr Ile Ile Ser Asp Pro Thr Cys Glu Ile Thr Gln Asn Thr Val Cys Ser
Pro Val Asp Val Asp Glu Met Cys Leu Leu Thr Val Arg Arg Thr Phe Asn Gly Ser Gly Thr
Tyr Cys Val Asn Leu Thr Leu Gly Asp Asp Thr Ser Leu Ala Leu Thr Ser Thr Leu Ile Ser
Val Pro Asp Arg Asp Pro Ala Ser

As used herein, the term “GPNMB activity” refers to one or more activities associated with GPNMB. To “modulate” GPNMB activity is to alter the baseline results observed with, and that can be attributed to GPNMB. To “neutralize” GPNMB is to cancel one or more effects, e.g. activity observed with, and that can be attributed to GPNMB.

As used herein, the term “isolated” refers to a molecule that is substantially free of its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

As used herein, the term “inhibit” or “inhibition of” refers to reducing by a measurable amount, or to prevent entirely.

As used herein, the term “Cytotoxic effect” in reference to the effect of an agent on a cell, means killing of the cell. “Cytostatic effect” refers to an inhibition of cell proliferation. A “cytotoxic agent” refers an agent that has a cytotoxic or cytostatic effect on a cell, thereby depleting or inhibiting the growth of, respectively, cells within a cell population.

As used herein (and unless the context indicates otherwise), the terms “prevent,” “preventing,” and “prevention” refer to the inhibition of the development or onset of a disorder associated with aberrant expression and/or activity of GPNMB (e.g., cancer) or the prevention of or otherwise delaying the recurrence, onset, or development of one or more symptoms of a disorder associated with aberrant expression and/or activity of GPNMB (e.g., cancer) in a subject resulting from the administration of a therapy or the administration of a combination of therapies.

As used herein, the term “effective amount” refers to a dosage or amount that is sufficient to result in amelioration of symptoms in a patient or to achieve a desired biological outcome.

As used herein, the term “prophylactically effective amount” refers to the amount of a therapy which is sufficient to result in the prevention of the development, recurrence, or onset of a disorder associated with aberrant expression and/or activity of GPNMB (e.g., cancer) or one or more symptoms thereof, or to enhance or improve the prophylactic effect(s) of another therapy.

As used herein, a “protocol” includes dosing schedules and dosing regimens. The protocols herein are methods of use and include prophylactic and therapeutic protocols.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, such as a cynomolgus monkey, chimpanzee, and a human), and more preferably a human.

As used herein (and unless the context indicates otherwise), the terms “therapeutic agent” and “therapeutic agents” normally refer to an agent that can be used in the prevention, treatment, management, or amelioration of a disorder associated with aberrant expression and/or activity of GPNMB (e.g., cancer) or one or more symptoms thereof. In certain embodiments, the term “therapeutic agent” refers to an antibody that immunospecifically binds to GPNMB. In certain other embodiments, the term “therapeutic agent” refers an agent other than an antibody that immunospecifically binds to GPNMB.

As used herein (and unless the context indicates otherwise), the terms “therapies” and “therapy” can refer to any protocol(s), method(s), and/or agent(s) that can be used in the prevention, treatment, management, or amelioration of a disorder associated with aberrant expression and/or activity of GPNMB (e.g., cancer) or one or more symptoms thereof. In certain embodiments, the terms “therapies” and “therapy” refer to anti-cancer therapy, biological therapy, supportive therapy, and/or other therapies useful in treatment, management, prevention, or amelioration of cancer or one or more symptoms thereof known to one of skill in the art such as medical personnel.

As used herein (and unless the context indicates otherwise), the terms “treat,” “treatment,” and “treating” normally refer to the eradication, removal, modification, or control of primary, regional, or metastatic cancer tissue, or the reduction or amelioration of the progression, severity, and/or duration of a disorder associated with aberrant expression and/or activity of GPNMB or amelioration of one or more symptoms thereof resulting from the administration of one or more therapies. In certain embodiments, such terms in the context of cancer refer to a reduction in the growth of cancerous cells, a decrease in number of cancerous cells and/or a reduction in the growth, formation and/or volume of a tumor. In other embodiments, such terms refer to the minimizing or delay of the spread of cancer resulting from the administration of one or more therapies to a subject with such a disease. Treatment can include, for example, a decrease in the severity of a symptom, the number of symptoms, or frequency of relapse.

### Nucleic Acids, Cloning and Expression Systems

Nucleic acids encoding the disclosed antibodies may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

The nucleic acids provided herein comprise a coding sequence for a CDR, a H variable domain, and/or a L variable domain disclosed herein.

The present disclosure also provides constructs in the form of plasmids, vectors, phagemids, transcription or expression cassettes which comprise at least one nucleic acid encoding a CDR, a H variable domain, and/or a L variable domain disclosed here.

The disclosure further provides a host cell comprising one or more constructs as above.

Also provided are nucleic acids encoding any CDR (CDR1, CDR2, CDR3 from either the H or L variable domain), H variable or L variable domain, as well as methods of making of the encoded products. The method comprises expressing the encoded product from the encoding nucleic acid. Expression may be achieved by culturing under appropriate conditions recombinant host cells containing the nucleic acid. Following production by expression, a H variable or L variable domain, or specific binding member may be isolated and/or purified using any suitable technique, then used as appropriate.

Antigen-binding fragments, H variable and/or L variable domains and encoding nucleic acid molecules and vectors may be isolated and/or purified from their natural environment, in substantially pure or homogeneous form, or, in the case of nucleic acid, free or substantially free of nucleic acid or genes of origin other than the sequence encoding a polypeptide with the required function.

Systems for cloning and expression of a polypeptide in a variety of different host cells are well known in the art including cells suitable for producing antibodies (Gene Expression Systems, Academic Press, eds. Fernandez et al., 1999). Briefly, suitable host cells include bacteria, plant cells, mammalian cells, and yeast and baculovirus systems. Mammalian cell lines available in the art for expression of a heterologous polypeptide include Chinese hamster ovary (CHO) cells, HeLa cells, baby hamster kidney cells, NS0 mouse myeloma cells, and many others. A common bacterial host is E. coli. Any protein expression system compatible with the invention may be used to produce the disclosed antibodies. Suitable expression systems also include transgenic animals (Gene Expression Systems, Academic Press, eds. Fernandez et al., 1999).

Suitable vectors can be chosen or constructed, so that they contain appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate (see Sambrook et al., Molecular Cloning: A Laboratory Manual, 2nd ed., Cold Spring Harbor Laboratory Press, 1989). Many known techniques and protocols for manipulation of nucleic acid, for example, in preparation of nucleic acid constructs, mutagenesis, sequencing, introduction of DNA into cells and gene expression, and analysis of proteins, are known in the art (Current Protocols in Molecular Biology, 2nd Edition, eds. Ausubel et al., John Wiley & Sons, 1992).

The invention also provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction of the nucleic acid into the cells may be followed by causing or allowing expression from the nucleic acid, e.g., by culturing host cells under conditions for expression of the gene.

### EXAMPLES

The following examples, including the experiments conducted and results achieved are provided for illustrative purposes only and are not to be construed as limiting upon the present invention.

The studies provided herein evaluate methods of targeting GPNMB in breast cancer patients. The studies presented herein utilized a toxin-conjugated anti-GPNMB antibody known as CDX-011vcMMAE (glembatumumab vedotin, CR011vcMMAE), but the methods are not to be limited to the antibody exemplified herein.

### Example 1

### Production of Human Anti-GPNMB Antibodies and Immunoconjugates

Human anti-GPNMB antibodies used in the studies herein were produced according to the procedures set forth in PCT Publication No. WO2006/071441. Conjugates of the human anti-GPNMB antibodies used in the studies herein were produced and tested according to the procedures set forth in PCT Publication No. WO2006/071441.

### Example 2

### Anti-GPNMB Therapy in Patients with Locally Advanced or Metastatic Breast Cancer

The studies presented herein were designed to evaluate the safety and efficacy of the anti-GPNMB antibody drug conjugate (CDX-011, CR011-vcMMAE) in patients with heavily pre-treated advanced breast cancer.

Immunohistochemistry for GPNMB was performed on patient biopsy samples from this clinical study using a polyclonal goat anti-GPNMB antibody (R&D Systems) and a biotin-conjugated donkey anti-goat secondary antibody (Jackson ImmunoResearch Laboratories). Sections were developed with DAB and counterstained with hematoxylin.

Patients with the following characteristics were considered eligible for the studies presented herein: locally advanced or metastatic breast cancer; female, age ≧18; progressive disease on study entry; Eastern Cooperative Oncology Group (ECOG) Performance Status 0-2 from the criteria shown below in Table 31; and had received at least two prior chemotherapy regimens, where hormonal therapy and biologic therapy do not count as one of the two prior regimens and where prior regimens must have included an anthracycline, a taxane, and capecitabine, as well as trastuzumab for HER-2/neu+disease, unless the patient was not a candidate for the agent. There was no limit to the number of prior regimens. The demographics of the patients are shown in Table 32, and the prior therapies for the patients are shown in Table 33.

TABLE 31
ECOG PERFORMANCE STATUS*

| | |
|---|---|
| 0 | Fully active, able to carry on all pre-disease performance |
| | without restriction |
| 1 | Restricted in physically strenuous activity but ambulatory and able to |
| | carry out work of a light or sedentary nature, e.g., light house work, |
| | office work |
| 2 | Ambulatory and capable of all selfcare but unable to carry out |
| | any work activities. Up and about more than 50% of waking hours |
| 3 | Capable of only limited selfcare, confined to bed or chair more |
| | than 50% of waking hours |
| 4 | Completely disabled. Cannot carry on any selfcare. Totally confined |
| | to bed or chair |
| 5 | Dead |

*As published in Am. J. Clin. Oncol.: Oken, M.M., Creech, R.H., Tormey, D.C.,
Horton, J., Davis, T.E., McFadden, E.T., Carbone, P.P.: Toxicity And Response Criteria Of The Eastern Cooperative Oncology Group. Am J Clin Oncol 5:649-655, 1982

TABLE 32
Patient Demographics

| Age | | |
|---|---|---|
| Median | 55 years | |
| Range | 33-69 | |
| ECOG Performance Status | n | % |
| 0 | 11 | 61% |
| 1 | 7 | 39% |
| 2 | 0 | 0% |
| Liver and/or Lung Metastases | | |
| Present | 15 | 83% |
| Absent | 3 | 7% |
| Estrogen Receptor | | |
| Positive | 9 | 50% |
| Negative | 9 | 50% |
| Progesterone Receptor | | |
| Positive | 7 | 39% |
| Negative | 11 | 61% |
| HER-2/neu | | |
| Positive | 3 | 17% |
| Negative | 14 | 78% |
| Not reported | 1 | 6% |
| ER negative/PR negative/HER-2 negative | 8 | 44% |

TABLE 33
Prior Therapy for Patients
Prior chemotherapy regimens in the metastatic setting

| Median | 4 | |
|---|---|---|
| Range | 2-11 | |
| | n | % |
| Taxane | 18 | 100 |
| Adjuvant/neoadjuvant setting | 8 | 44 |
| Metastatic setting | 17 | 94 |
| Anthracycline | 15 | 83 |
| Adjuvant/neoadjuvant setting | 11 | 61 |
| Metastatic setting | 4 | 22 |
| Capecitabine | | |
| Metastatic setting | 18 | 100 |
| Other | 18 | 100 |
| Gemcitabine | 12 | 67 |
| Bevacizumab | 10 | 56 |
| Vinorelbine | 8 | 44 |
| Epothilone | 4 | 22 |
| Trastuzumab | 4 | 22 |

For Phase I dose escalation studies, the starting dose was 1.34 mg/kg IV every three weeks (q3w). Sequential dose cohorts (n=3) were enrolled based on tolerability. The cap was set at 1.88 mg/kg IV q3w.

In the preliminary studies, 18 patients were treated, and ultimately four continued on in the study, and treatment was discontinued in 14 patients (12 for progression of the disease, two for an adverse event, one for neuropathy and one for rash. The first two patients enrolled at 1.34 mg/kg had dose-limiting worsening of peripheral sensory neuropathy. Both patients had baseline neuropathy. Subsequently, patients with baseline grade 2 or higher neuropathy were excluded. The dose escalation study was re-started at 1.00 mg/kg IV q3w. 1.88 mg/kg IV q3w was tolerated and selected for further study.

The toxicity results are shown below in Table 34. All treatment-emergent adverse events are shown in the table, regardless of attribution.

TABLE 34
Toxicity Profile

| CTCAE Category/ | Total | Grade 3 | Grade 4 |
|---|---|---|---|
| Term | n (%) | n (%) | n (%) |
| Any Event | 18 (100%) | 10 (56%) | 0 (0%) |

Dermatology/Skin

| | | | |
|---|---|---|---|
| Rash | 11 (61%) | 1 (6%) | 0 (0%) |
| Alopecia | 9 (50%) | — | — |
| Pruritus | 3 (17%) | 0 (0%) | 0 (0%) |

Constitutional Symptoms

| | | | |
|---|---|---|---|
| Fatigue | 9 (50%) | 1 (6%) | 0 (0%) |
| Fever | 5 (28%) | 0 (0%) | 0 (0%) |

Gastrointestinal

| | | | |
|---|---|---|---|
| Nausea | 8 (44%) | 1 (6%) | 0 (0%) |
| Vomiting | 7 (39%) | 0 (0%) | 0 (0%) |
| Constipation | 6 (33%) | 0 (0%) | 0 (0%) |
| Diarrhea | 5 (28%) | 0 (0%) | 0 (0%) |
| Anorexia | 5 (28%) | 0 (0%) | 0 (0%) |
| Dysgeusia | 4 (22%) | 0 (0%) | 0 (0%) |

Neurology

| | | | |
|---|---|---|---|
| Neuropathy | 7 (39%) | 2 (11%) | 0 (0%) |
| Dizziness | 4 (22%) | 0 (0%) | 0 (0%) |

Pain

| | | | |
|---|---|---|---|
| Gastrointestinal | 6 (33%) | 1 (6%) | 0 (0%) |
| Musculoskeletal | 5 (28%) | 1 (6%) | 0 (0%) |

Pulmonary

| | | | |
|---|---|---|---|
| Cough | 6 (33%) | 1 (6%) | 0 (0%) |

Blood/Bone Marrow

| | | | |
|---|---|---|---|
| Neutropenia | 5 (28%) | 3 (17%) | 0 (0%) |

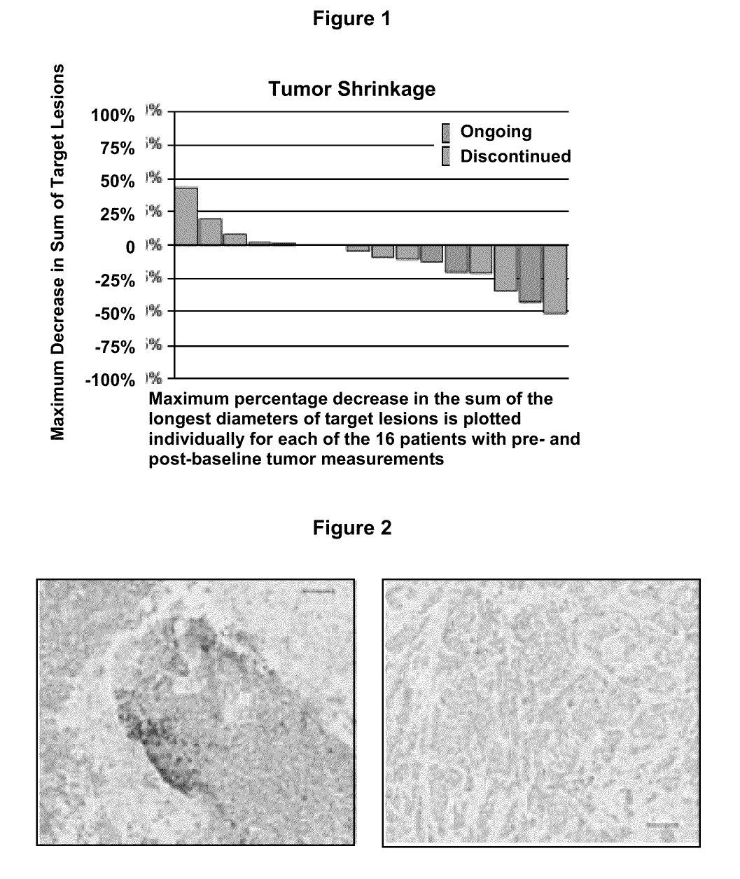

Of the 18 patients, three had at least a partial response, with one confirmed, and tumor shrinkage was observed in 9 patients (50%). The level of tumor shrinkage is shown in FIG. 1.

In addition, the following activity was seen in individual patients. One patient, a 56 year old woman with ER+/PR+/HER2+ breast cancer, with metastatic disease and who had received hormonal therapies and 11 prior chemotherapy regimens in the metastatic setting, including various combinations of paclitaxel, docetaxel, cyclophosphamide, adriamycin, capecitabine, trastuzumab, vinorelbine, gemcitabine, carboplatin, and two investigational agents, had soft tissue and bony metastases on study entry. CT scans demonstrated partial response after two cycles of CR011-vcMMAE and confirmed 6 weeks later. After treatment for over 5 months, this patient had shown a maximum tumor reduction of 42%.

A second patient, a 39 year old woman with ER+/PR+/HER2− breast cancer, with metastatic disease and who had received previous regimens in the metastatic setting of paclitaxel/bevacizumab, capecitabine, and tamoxifen, had liver, lung and bone metastases at study entry. Tumor biopsy was positive for GPNMB expression. CT scans demonstrated partial response (51% reduction in target lesions) after two cycles of CR011-vcMMAE. The patient was discontinued from study 6 weeks later after restaging revealed tumor growth.

A third patient, a 69 year old woman with triple negative (ER−/PR−/HER2−) breast cancer, with metastatic disease since 2006 and who had previously received combinations of paclitaxel, bevacizumab, capecitabine, cisplatin, gemcitabine, Abraxane, and ixabepilone, had hepatic metastases and a pleural effusion at study entry. CT scans demonstrated partial response (34% reduction in target lesions) after four cycles of CR011-vcMMAE. Approximately 9 weeks later, the patient was hospitalized with cough and dyspnea and was discontinued after 23 weeks on study.

A third patient, a 41 year old woman with triple negative (ER−/PR−/HER2−) breast cancer with metastatic disease and who had received bevacizumab, Abraxane, gemcitabine, capecitabine, and tamoxifen, presented with disease in the liver and bone including skull metastases associated with paresis of the mental nerve and pain requiring narcotic analgesics. Following two cycles of CR011-vcMMAE, the patient had marked clinical improvement with resolution of mental nerve paresis and discontinuation of analgesics. CT scan showed mixed results with some regression in hepatic lesions and two new small lesions, thought to be “inflammatory”. Bone scan and MRI were unchanged. The patient continued to receive treatment with CR011-vcMMAE for a total of 17 weeks.

Immunohistochemistry for GPNMB was been performed in five patient tumor samples. One sample was positive (FIG. 2, left panel), one had a small area of focal positivity, and three were negative (representative image shown in FIG. 2, right panel).

For the Phase II Simon Two-Stage study, the primary endpoint was set at progression-free rate at 12 weeks. The following statistical assumptions were used p₀=10%; p₁=30%; α=β=0.10; first stage included sixteen patients; and if two or more patients were progression-free at 12 weeks, the total enrollment would increase to 25. The primary efficacy endpoint was met as at least 9 out of 26 patients were found to be without progression at 12 weeks.

The patient characteristics and prior therapies for the patients in this Phase II study are shown in Tables 35 and 36, respectively.

TABLE 35
Patient Characteristics

| Characteristic | | All Patients (n = 42) |
|---|---|---|
| Cancer Stage (n [%]) | III | 2 (5%) |
| | IV | 40 (95%) |
| Age (median [range]) | | 58 (33-76) |
| ECOG Performance | 0 | 21 (50%) |
| Status (n [%]) | 1 | 19 (45%) |
| | 2 | 2 (5%) |
| Metastatic to liver and/or | | 34 (83%) |
| lung (n [%]) | | |
| Receptor status | ER+ | 24 (57%) |
| (n [%]) | PR+ | 15 (36%) |
| | HER-2/neu+ | 11 (27%)† |
| | Triple-negative | 13 (32%)† |
| | (ER−/PR−/HER-2/neu−) | |

†HER-2/neu status is unknown for one patient..

TABLE 36
Prior Therapies

| Prior Therapies | |
|---|---|
| Prior chemotherapy regimens | All Patients (n = 42) |
| (median [range]) | 7 (2-18) |
| Taxane | 42 (100%) |
| Capecitabine | 41 (98%) |
| Anthracycline | 38 (90%) |
| Hormonal Therapy | 30 (71%) |
| Gemcitabine | 27 (64%) |
| Bevacizumab | 24 (57%) |
| Vinorelbine | 23 (55%) |
| Epothilone | 17 (40%) |
| Trastuzumab | 14 (33%) |
| Lapatinib | 8 (19%) |
| Investigational agents | 8 (19%) |

The tolerability of the CR011-vcMMAE (CDX-011) treatment was evaluated in these patients. The adverse events potentially related to CDX-011 treatment are shown in Table 37. In particular, the treated patients (n=42) received a mean of 3.5 (range 1-8) cycles of treatment during this study. Four patients discontinued treatment due to adverse events (neuropathy, rash, dermatologic bullae and acute renal failure). Dose-escalation dose-limiting toxicities (DLTs) were limited to two cases of neuropathy (at the 1.34 mg/kg dose). After revision of the protocol to exclude pre-existing neuropathy ≧Grade 2, no further DLT occurred. Serious adverse events potentially related to CDX-011 (all single cases at the Phase II dose) included intractable vomiting/nausea, dermatologic bullae and acute renal failure.

TABLE 37
Tolerability Profiles

| Adverse events | All Patients (n = 42) | |
|---|---|---|
| potentially related | Total | Grade 3 |
| to CDX-011 | n (%) | n (%) |
| Fatigue | 20 (48%) | 2 (5%) |
| Rash | 19 (45%) | 2 (5%) |
| Nausea | 19 (45%) | 2 (5%) |
| Alopecia | 14 (33%) | — |
| Neutropenia | 12 (29%) | 8 (19%) |
| Vomiting | 12 (29%) | 1 (2%) |
| Neuropathy | 10 (24%) | 1 (2%) |
| Anemia | 10 (24%) | — |
| Asthenia | 3 (7%) | 2 (5%) |

Table includes events potentially related to treatment, occurring in ≧ 20% of patients overall or at Grade 3 severity in ≧ 5%. There were no Grade 4/5 treatment-related events.

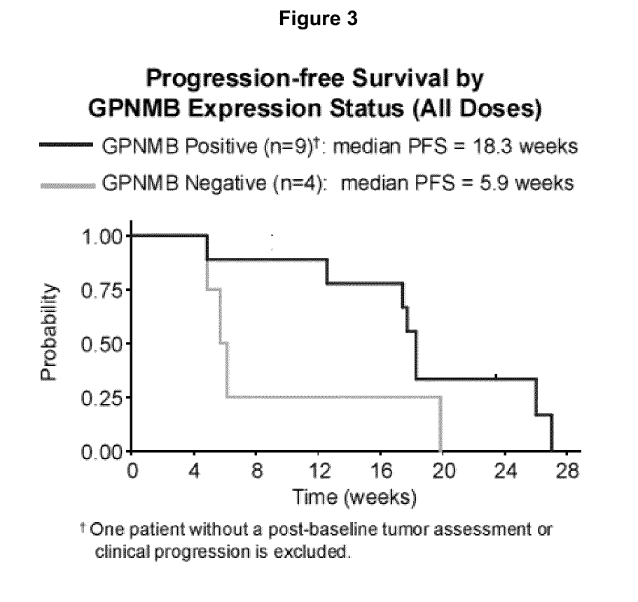

Immunohistochemistry for GPNMB expression was performed on biopsy samples for a subset of patients using a polyclonal goat anti-GPNMB antibody (R&D Systems) and a biotin-conjugated donkey anti-goat secondary antibody (Jackson ImmunoResearch Laboratories). Sections were developed with DAB and counterstained with hematoxylin. Samples with 5% of cells expressing GPNMB were considered positive. The results of this analysis are shown below in Table 38 and in FIG. 3.

TABLE 38
Immunohistochemistry Analysis

| | Analyzed | Positive |
|---|---|---|
| All Doses | n | n (%) |
| Overall | 14 | 10 (71%) |
| Triple-negative (ER−/PR−/HER-2/neu−) | 7 | 5 (71%) |
| Partial Response (PR) | 3 | 3 (100%) |
| Stable Disease (SD) or better | 9 | 8 (89%) |
| Progression-free at 12 weeks | 8 | 7 (88%) |

One patient with triple-negative disease exhibited strongly positive stromal expression of GPNMB at study entry, and partial response (53% shrinkage) was maintained for 23+ weeks in this patient.

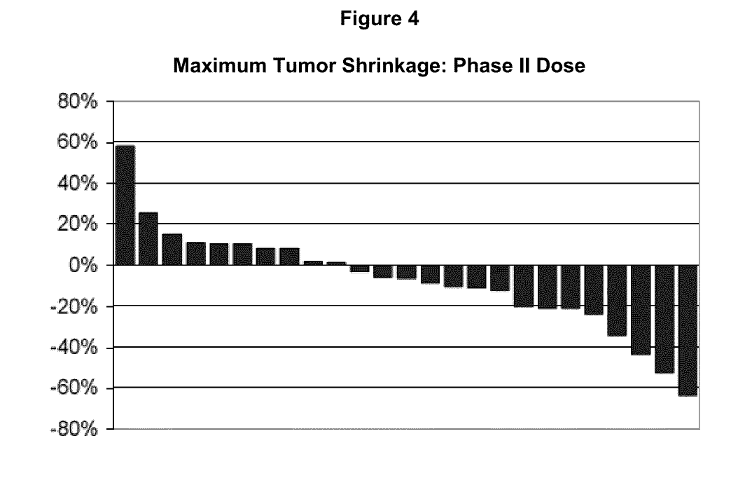

The maximum tumor shrinkage for the patients in these studies is shown in FIG. 4. Maximum percent decrease in the sum of longest diameters of target lesions is plotted individually for all patients treated at the Phase II dose (1.88 mg/kg), including dose-escalation (n=6) and Phase II (n=28). This graph excludes nine patients without post-baseline imaging of measurable lesions.

Additional measures of anti-tumor activity were evaluated for the patients in this further study. These additional measures are shown in Table 39.

TABLE 39
Additional Measures of Anti-tumor Activity at Phase II Dose
Additional Measures of Anti-tumor Activity: Phase II Dose

| | Phase II dose | Triple-negative | |
|---|---|---|---|
| | 1.88 mg/kg | disease | Expressing GPNMB |
| Best Response (RECIST criteria) | | | |
| Partial Response (PR) | 4/32 (13%) † | 2/10 (20%) | 2/7 (29%) ** |
| Confirmed PR | 2/32 (6%) | 1/10 (10%) | 2/7 (29%) |
| Stable Disease (SD) or better | 18/32 (56%) † | 8/10 (80%) | 7/7 (100%) ** |
| Any tumor shrinkage | 15/25 (60%) †‡ | 7/9 (78%) * | 5/7 (71%) ** |
| Median PFS (weeks) | 9.1 | 17.9 | 18.3 |

† Patients treated at the Phase II dose (1.88 mg/kg), including dose-escalation (n = 6) and Phase II (n = 28). Two patients without a post-baseline tumor assessment or clinical progression are excluded.
‡ Excludes an additional seven patients without post-baseline imaging of measurable lesions.
* Ten triple-negative patients were treated at the Phase II dose. One patient without post-baseline imaging of measurable lesions is excluded.
** Eight GPNMB-expressing patients were treated at-the Phase II dose. Of these, one without post-baseline tumor assessment or clinical progression is excluded.

Thus, the studies presented herein demonstrate that CR011-vcMMAE administered at 1.88 mg/kg IV q3w is well-tolerated in patients with advanced breast cancer. Tumor shrinkage, including partial responses, palliation of bone pain, and stable disease have been observed in heavily-pretreated patients, including some with triple-negative disease. Toxicity in patients with breast cancer is similar to that observed in patients with melanoma. Rash is the most common adverse event reported in patients treated with CR011-vcMMAE. GPNMB, the target of CR011-vcMMAE, is specifically expressed in breast cancer tissue as observed in a microarray comprising over 500 core samples. The target GPNMB was frequently expressed (71%) in this patient population of advanced breast cancer patients who were heavily pretreated (median of seven prior regimens), and expression of GPNMB was associated with improved outcomes following treatment with CDX-011. All activity parameters appear to be improved for CDX-011-treated patients expressing GPNMB Thus, therapies that target GPNMB are useful in treating breast cancer in patients with locally advanced or metastatic breast cancer, particularly in the subset of patients with triple-negative disease where treatment options are relatively limited.

### EQUIVALENTS

The foregoing description and Examples detail certain preferred embodiments of the antibodies and describes the best mode contemplated by the inventors. It will be appreciated, however, that no matter how detailed the foregoing may appear in text, the methods of making and using the antibodies described herein may be practiced in many ways. The foregoing written specification is considered to be sufficient to enable one skilled in the art to practice the embodiments described herein.

What is claimed is:

1. A method for treating, delaying the progression of, alleviating a symptom of, or otherwise ameliorating breast cancer in a subject in need thereof, comprising administering an isolated antibody that specifically binds to GPNMB or a conjugate of such an antibody.

2. A method as claimed in claim 1 wherein the breast cancer is an estrogen receptor-negative, progesterone receptor-negative or human epidermal growth factor receptor 2 (HER2) negative breast cancer.

3. A method for treating, delaying the progression of, alleviating a symptom of, or otherwise ameliorating breast cancer in a subject who is non-responsive or no longer responsive to a previous treatment, comprising administering an isolated antibody that specifically binds to GPNMB or a conjugate of such an antibody.

4. A method for treating, delaying the progression of, alleviating a symptom of, or otherwise ameliorating basal-like or triple-negative (TN) breast cancer in a subject in need thereof, comprising administering an isolated antibody that specifically binds to GPNMB or a conjugate of such an antibody.

5. The method of claim 1, wherein the breast cancer is metastatic.

6. The method of claim 1 comprising the further step of first detecting GPNMB expression or overexpression in a biological sample from said subject.

7. The method of claim 1, wherein the antibody is a monoclonal antibody.

8. The method of claim 1, wherein the antibody is a human antibody.

9. The method of claim 1, wherein the antibody is a fully human antibody.

10. The method of claim 1, wherein the antibody specifically binds GPNMB with an affinity constant greater than 10⁷M⁻¹.

11. The method of claim 1, wherein the antibody comprises a region selected from the group comprising: VH1-2, VH2-5, VH3-11, VH3-21, VH3-30, VH3-33, VH4-31, VH4-59 and VH5-51 or a region derived from a region selected from the group comprising: VH1-2, VH2-5, VH3-11, VH3-21, VH3-30, VH3-33, VH4-31, VH4-59 and VH5-51.

12. The method of claim 1, wherein the antibody comprises a region selected from the group comprising: A2, A3, A20, A27, A30, L2 and O1 or a region derived from a region selected from the group comprising: A2, A3, A20, A27, A30, L2 and O1.

13. The method of claim 1, wherein the antibody comprises an amino acid sequence selected from the group comprising: SEQ ID NO: 2, 20, 38, 56, 74, 92, 110, 128, 146, 164, 182, 200, 218, 236 and 254.

14. The method of claim 1, wherein the antibody comprises an amino acid sequence selected from the group comprising: SEQ ID NO: 11, 29, 47, 65, 83, 101, 119, 137, 155, 173, 191, 209, 227, 245 and 263.

15. The method of claim 1, wherein the antibody comprises:
(a) a V_{H} CDR1 region comprising the amino acid sequence of SEQ ID NO: 22, 4, 40, 58, 76, 94, 112, 130, 148, 166, 184, 202, 220, 238 or 256;
(b) a V_{H} CDR2 region comprising the amino acid sequence of SEQ ID NO: 24, 5, 42, 60, 78, 96, 114, 132, 150, 168, 186, 204, 222, 240 or 258;
(c) a V_{H} CDR3 region comprising the amino acid sequence of SEQ ID NO: 26, 8, 44, 62, 80, 98, 116, 134, 152, 170, 188, 206, 224, 242 or 260,
(d) a V_{L} CDR1 region comprising the amino acid sequence of SEQ ID NO: 31, 13, 49, 67, 85, 103, 121, 139, 157, 175, 193, 211, 229, 247 or 265;
(e) a V_{L} CDR2 region comprising the amino acid sequence of SEQ ID NO: 33, 15, 51, 69, 87, 105, 123, 141, 159, 177, 195, 213, 231, 249 or 267; and
(f) a V_{L} CDR3 region comprising the amino acid sequence of SEQ ID NO: 35, 17, 53, 71, 89, 107, 135, 143, 161, 179, 197, 215, 233, 251 or 269.

16. The method of claim 1, wherein the antibody is selected from the group comprising: Mab 1.10.2, Mab 1.15.1, Mab 1.2.2, Mab 1.7.1, Mab 2.10.2, Mab 2.15.1, Mab 2.16.1, Mab 2.17.1, Mab 2.21.2, Mab 2.22.1, Mab 2.24.1, Mab 2.3.1, Mab 2.7.1 and Mab 2.8.1.

17. The method of claim 1, wherein said antibody is an IgG1 or IgG2 antibody.

18. The method of claim 1, wherein the antibody is in the form of an immunoconjugate comprising an antibody as defined in any one of claim 13 to 16 and a cytotoxic agent.

19. The method of claim 18, wherein the cytotoxic agent is auristatin E (dolastatin-10) or a derivative thereof.

20. The method of claim 19, in which said antibody is Mab 1.15.1.

21. The method of claim 20, in which the immunoconjugate is glembatumumab vedotin.

22. The method of claim 1, wherein said subject is human.

23. The method of claim 1, further comprising administering to said subject the immunoconjugate of claim 18, and wherein said effective amount is a unit dose between 0.1 mg/kg to 10 mg/kg, with 2 to 4 administrations.

24. The method of claim 1, wherein said effective amount is a unit dose between 0.1 mg/kg to 2 mg/kg.

25. The method of claim 1, wherein said effective amount is a unit dose about 1 mg/kg.

26. The method of claim 1, wherein the antibody or conjugate is administered in an 18 to 25 day cycle.

27. The method of claim 1, wherein the antibody or conjugate is administered in a 21 day cycle.

28. The method of claim 1, wherein the treatment is in a adjuvant setting.

29. The method of claim 1, wherein the treatment is in a neoadjuvant setting.

30. The method of claim 1, wherein the treatment is in a metastatic setting.

31. The method of claim 1, wherein the antibody or conjugate is administered parenterally.

32. The method of claim 1, wherein the antibody or conjugate is administered intravenously.


SEQUENCE LISTING (272 sequences)

SEQ ID NO: 1 - DNA, 347 bp, Homo sapiens
aggtgcagctgcaggagtcgggcccaggactggtgaagccttcggagaccctgtccctcacctgcactgtctctggtgactccatcagtaattactactggagctggatccggcagcccccagggaagggactggagtggattgggtatttctattacagtgggagcaccaactacaacccctccctcaagagtcgagtcaccatatcagtagacacgtccaagaaccagttctccctgaaactgagctctgtgaccgctgcggacacggccgtgtattactgtgcgagagataggggctgggctgactactggggccagggaaccctggtcaccgtctcctcagcc

SEQ ID NO: 2 - protein, 116 aa, Homo sapiens
QVQLQESGPGLVKPSETLSLTCTVSGDSISNYYWSWIRQPPGKGLEWIGYFYYSGSTNYNPSLKSRVTISVDTSKNQFSLKLSSVTAADTAVYYCARDRGWADYWGQGTLVTVSSA

SEQ ID NO: 3 - protein, 25 aa, Homo sapiens
QVQLQESGPGLVKPSETLSLTCTVS

SEQ ID NO: 4 - protein, 10 aa, Homo sapiens
GDSISNYYWS

SEQ ID NO: 5 - protein, 14 aa, Homo sapiens
WIRQPPGKGLEWIG

SEQ ID NO: 6 - protein, 16 aa, Homo sapiens
YFYYSGSTNYNPSLKS

SEQ ID NO: 7 - protein, 32 aa, Homo sapiens
RVTISVDTSKNQFSLKLSSVTAADTAVYYCAR

SEQ ID NO: 8 - protein, 7 aa, Homo sapiens
DRGWADY

SEQ ID NO: 9 - protein, 12 aa, Homo sapiens
WGQGTLVTVSSA

SEQ ID NO: 10 - DNA, 324 bp, Homo sapiens
gaaattgtgttgacgcagtctccaggcaccctgtctttgtctccaggggaaagggccaccctctcctgcagaaccagtcagagtattagcagcagctatttagcctggtaccagcagaaacctggccaggttcccaggctcctcatctatggtgcttccagcagggccactggcatcccagacaggttcagtggcagtgggtctgggacagacttcactctcaccatcagcagactggagcctgaagattttgcagtgtattattgtcagcagtatggtagctcgatcaccttcggccaagggacacgactggagattaaacga

SEQ ID NO: 11 - protein, 108 aa, Homo sapiens
EIVLTQSPGTLSLSPGERATLSCRTSQSISSSYLAWYQQKPGQVPRLLIYGASSRATGIPDRFSGSGSGTDFTLTISRLEPEDFAVYYCQQYGSSITFGQGTRLEIKR

SEQ ID NO: 12 - protein, 23 aa, Homo sapiens
EIVLTQSPGTLSLSPGERATLSC

SEQ ID NO: 13 - protein, 12 aa, Homo sapiens
RTSQSISSSYLA

SEQ ID NO: 14 - protein, 15 aa, Homo sapiens
WYQQKPGQVPRLLIY

SEQ ID NO: 15 - protein, 7 aa, Homo sapiens
GASSRAT

SEQ ID NO: 16 - protein, 32 aa, Homo sapiens
GIPDRFSGSGSGTDFTLTISRLEPEDFAVYYC

SEQ ID NO: 17 - protein, 8 aa, Homo sapiens
QQYGSSIT

SEQ ID NO: 18 - protein, 11 aa, Homo sapiens
FGQGTRLEIKR

SEQ ID NO: 19 - DNA, 360 bp, Homo sapiens
caggtgcagctgcaggagtcgggcccaggactggtgaagccttcacagaccctgtccctcacctgcactgtctctggtggctccatcagcagttttaattactactggagctggatccgccaccacccagggaagggcctggagtggattgggtacatctattacagtgggagcacctactccaacccgtccctcaagagtcgagttaccatatcagtagacacgtctaagaaccagttctccctgacgctgagctctgtgactgccgcggacacggccgtgtattactgtgcgagagggtataactggaactactttgactactggggccagggaaccctggtcaccgtctcctcagcc

SEQ ID NO: 20 - protein, 120 aa, Homo sapiens
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSA

SEQ ID NO: 21 - protein, 30 aa, Homo sapiens
QVQLQESGPGLVKPSQTLSLTCTVSGGSIS

SEQ ID NO: 22 - protein, 7 aa, Homo sapiens
SFNYYWS

SEQ ID NO: 23 - protein, 14 aa, Homo sapiens
WIRHHPGKGLEWIG

SEQ ID NO: 24 - protein, 16 aa, Homo sapiens
YIYYSGSTYSNPSLKS

SEQ ID NO: 25 - protein, 32 aa, Homo sapiens
RVTISVDTSKNQFSLTLSSVTAADTAVYYCAR

SEQ ID NO: 26 - protein, 9 aa, Homo sapiens
GYNWNYFDY

SEQ ID NO: 27 - protein, 12 aa, Homo sapiens
WGQGTLVTVSSA

SEQ ID NO: 28 - DNA, 327 bp, Homo sapiens
gaaatagtgatgacgcagtctccagccaccctgtctgtgtctccaggggaaagagccaccctctcctgcagggccagtcagagtgttgacaacaacttagtctggtaccagcagaaacctggccaggctcccaggctcctcatctatggtgcatccaccagggccactggtatcccagccaggttcagtggcagtgggtctgggacagagttcactctcaccatcagtagtctgcagtctgaagattttgcagtttattactgtcagcagtataataactggcctccgtggacgttcggccaagggaccaaggtggaaatcaaacga

SEQ ID NO: 29 - protein, 109 aa, Homo sapiens
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKR

SEQ ID NO: 30 - protein, 23 aa, Homo sapiens
EIVMTQSPATLSVSPGERATLSC

SEQ ID NO: 31 - protein, 11 aa, Homo sapiens
RASQSVDNNLV

SEQ ID NO: 32 - protein, 15 aa, Homo sapiens
WYQQKPGQAPRLLIY

SEQ ID NO: 33 - protein, 7 aa, Homo sapiens
GASTRAT

SEQ ID NO: 34 - protein, 32 aa, Homo sapiens
GIPARFSGSGSGTEFTLTISSLQSEDFAVYYC

SEQ ID NO: 35 - protein, 10 aa, Homo sapiens [sequence omitted]

SEQ ID NO: 36 - protein, 11 aa, Homo sapiens
FGQGTKVEIKR

SEQ ID NO: 37 - DNA, 369 bp, Homo sapiens
atcaccttgaaggagtctggtcctacgctggtgaaacccacacagaccctcacgctgacctgcaccttctctgggttctcactcagcgctggtggagtgggtgtgggctggatccgtcagcccccaggaaaggccctggagtggcttgcactcatttattggaatgatgataagcgctacagcccatctctgaggagcaggctcaccatcaccaaggacacctccaaaaaccaggtggtccttacaattaccaacatggaccctgtggacacagccacatattattgtgcacacagtcactatgattacgattgggggagttactttgactactggggccagggaaccctggtcaccgtctcctcagcc

SEQ ID NO: 38 - protein, 123 aa, Homo sapiens
ITLKESGPTLVKPTQTLTLTCTFSGFSLSAGGVGVGWIRQPPGKALEWLALIYWNDDKRYSPSLRSRLTITKDTSKNQVVLTITNMDPVDTATYYCAHSHYDYDWGSYFDYWGQGTLVTVSSA

SEQ ID NO: 39 - protein, 24 aa, Homo sapiens
ITLKESGPTLVKPTQTLTLTCTFS

SEQ ID NO: 40 - protein, 12 aa, Homo sapiens
GFSLSAGGVGVG

SEQ ID NO: 41 - protein, 14 aa, Homo sapiens
WIRQPPGKALEWLA

SEQ ID NO: 42 - protein, 16 aa, Homo sapiens
LIYWNDDKRYSPSLRS

SEQ ID NO: 43 - protein, 32 aa, Homo sapiens
RLTITKDTSKNQVVLTITNMDPVDTATYYCAH

SEQ ID NO: 44 - protein, 13 aa, Homo sapiens
SHYDYDWGSYFDY

SEQ ID NO: 45 - protein, 12 aa, Homo sapiens
WGQGTLVTVSSA

SEQ ID NO: 46 - DNA, 342 bp, Homo sapiens
gatattgtgatgacccagactccactctccctgcccgtcacccctggagagccggcctccatctcctgcaggtctagtcagagcctcttggatagtgatgatggaaacacctatttggactggtacctgcagaagccaggacagtctccacagctcctgatctatacgctttcctatcgggcctctggagtcccagacaggttcagtggcagtgggtcaggcactgatttcacactgaacatcagcagggtggaggctgaggatgttggagtttattactgcatgcaacgtatagagtttcctatcaccttcggccaagggacacgactggagattaaacga

SEQ ID NO: 47 - protein, 114 aa, Homo sapiens
DIVMTQTPLSLPVTPGEPASISCRSSQSLLDSDDGNTYLDWYLQKPGQSPQLLIYTLSYRASGVPDRFSGSGSGTDFTLNISRVEAEDVGVYYCMQRIEFPITFGQGTRLEIKR

SEQ ID NO: 48 - protein, 23 aa, Homo sapiens
DIVMTQTPLSLPVTPGEPASISC

SEQ ID NO: 49 - protein, 17 aa, Homo sapiens
RSSQSLLDSDDGNTYLD

SEQ ID NO: 50 - protein, 15 aa, Homo sapiens
WYLQKPGQSPQLLIY

SEQ ID NO: 51 - protein, 7 aa, Homo sapiens
TLSYRAS

SEQ ID NO: 52 - protein, 32 aa, Homo sapiens
GVPDRFSGSGSGTDFTLNISRVEAEDVGVYYC

SEQ ID NO: 53 - protein, 9 aa, Homo sapiens
MQRIEFPIT

SEQ ID NO: 54 - protein, 11 aa, Homo sapiens
FGQGTRLEIKR

SEQ ID NO: 55 - DNA, 360 bp, Homo sapiens
caggtgcagctgcaggagtcgggcccaggactggtgaagccttcacagaccctgtccctcacctgcactgtctctggtggctccatcagcagtgctaattactactggacctggatccgccagcacccagggaagggcctggagtggattgggtacatctattacagtgggagcacctactgcaacccgtccctcaagagtcgagttatcatatcagtagacacgtctaagaaccagttctccctgaagctgagctctgtgactgccgcggacacggccgtgtattactgtgcgagagggtataactggaactactttgactactggggccagggaaccctggtcaccgtctcctcagcc

SEQ ID NO: 56 - protein, 120 aa, Homo sapiens
QVQLQESGPGLVKPSQTLSLTCTVSGGSISSANYYWTWIRQHPGKGLEWIGYIYYSGSTYCNPSLKSRVIISVDTSKNQFSLKLSSVTAADTAVYYCARGYNWNYFDYWGQGTLVTVSSA

SEQ ID NO: 57 - protein, 24 aa, Homo sapiens
QVQLQESGPGLVKPSQTLSLTCTV

SEQ ID NO: 58 - protein, 12 aa, Homo sapiens
GGSISSANYYWT

SEQ ID NO: 59 - protein, 14 aa, Homo sapiens
WIRQHPGKGLEWIG

SEQ ID NO: 60 - protein, 16 aa, Homo sapiens
YIYYSGSTYCNPSLKS

SEQ ID NO: 61 - protein, 32 aa, Homo sapiens
RVIISVDTSKNQFSLKLSSVTAADTAVYYCAR

SEQ ID NO: 62 - protein, 9 aa, Homo sapiens
GYNWNYFDY

SEQ ID NO: 63 - protein, 12 aa, Homo sapiens
WGQGTLVTVSSA

SEQ ID NO: 64 - DNA, 330 bp, Homo sapiens
gatatagtgatgacgcagtctccagccaccctgtctgtgtctccaggggaaagagccaccctctcctgcagggccagtcagagtgttagcagcaacttagcctggtaccaggagagacctggccaggctcccagactcctcatctatggtgcatccaccagggccactggtatcccagccaggttcagtggcagtgggtctgggacagagttcactctcaccatcagcagcctgcagtctgaagattttgcagtttattactgtcagcagtataataagtggcctccgtggacgttcggccaagggaccaaggtggaaatcgaacgaact

SEQ ID NO: 65 - protein, 109 aa, Homo sapiens
DIVMTQSPATLSVSPGERATLSCRASQSVSSNLAWYQERPGQAPRLLIYGASTRATGIPARFSGSGSGTEFTLTISSLQSEDFAVYYCQQYNKWPPWTFGQGTKVEIER

SEQ ID NO: 66 - protein, 23 aa, Homo sapiens
DIVMTQSPATLSVSPGERATLSC

SEQ ID NO: 67 - protein, 11 aa, Homo sapiens
RASQSVSSNLA

SEQ ID NO: 68 - protein, 15 aa, Homo sapiens
WYQERPGQAPRLLIY

SEQ ID NO: 69 - protein, 7 aa, Homo sapiens
GASTRAT

SEQ ID NO: 70 - protein, 32 aa, Homo sapiens; identical to SEQ ID NO: 34

SEQ ID NO: 71 - protein, 10 aa, Homo sapiens
QQYNKWPPWT

SEQ ID NO: 72 - protein, 11 aa, Homo sapiens
FGQGTKVEIER

SEQ ID NO: 73 - DNA, 378 bp, Homo sapiens
cagctggtggagtctgggggaggcgtggtccagcctgggaggtccctgagactctcctgtgcagcctctggattcgccttcagtagctatggcatgcactgggtccgccaggctccaggcaaggggctggagtgggtggcagttatatcatatgatggaaataataaatactatgcagactccgtgaagggccgattcaccatctccagagacaattccaagaacacgctgtatctgcaaatgaacagcctgagagctgaggacacggctgtgtattactgtgcgagagatctagtggttcggggaattagggggtactactactacttcggtatggacgtctggggccaagggaccacggtcaccgtctcctcagcc

SEQ ID NO: 74 - protein, 126 aa, Homo sapiens
QLVESGGGVVQPGRSLRLSCAASGFAFSSYGMHWVRQAPGKGLEWVAVISYDGNNKYYADSVKGRFTISRDNSKNTLYLQMNSLRAEDTAVYYCARDLVVRGIRGYYYYFGMDVWGQGTTVTVSSA

SEQ ID NO: 75 - protein, 23 aa, Homo sapiens
QLVESGGGVVQPGRSLRLSCAAS

SEQ ID NO: 76 - protein, 10 aa, Homo sapiens
GFAFSSYGMH

SEQ ID NO: 77 - protein, 14 aa, Homo sapiens
WVRQAPGKGLEWVA

SEQ ID NO: 78 - protein, 17 aa, Homo sapiens
VISYDGNNKYYADSVKG

SEQ ID NO: 79 - protein, 32 aa, Homo sapiens
RFTISRDNSKNTLYLQMNSLRAEDTAVYYCAR

SEQ ID NO: 80 - protein, 18 aa, Homo sapiens
DLVVRGIRGYYYYFGMDV

SEQ ID NO: 81 - protein, 12 aa, Homo sapiens
WGQGTTVTVSSA

SEQ ID NO: 82 - DNA, 339 bp, Homo sapiens
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaggtctacaaactccgatcaccttcggccaagggacacgactggagattaaacga

SEQ ID NO: 83 - protein, 113 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPITFGQGTRLEIKR

SEQ ID NO: 84 - protein, 23 aa, Homo sapiens
DIVMTQSPLSLPVTPGEPASISC

SEQ ID NO: 85 - protein, 16 aa, Homo sapiens
RSSQSLLHSNGYNYLD

SEQ ID NO: 86 - protein, 15 aa, Homo sapiens
WYLQKPGQSPQLLIY

SEQ ID NO: 87 - protein, 7 aa, Homo sapiens
LGSNRAS

SEQ ID NO: 88 - protein, 32 aa, Homo sapiens
GVPDRFSGSGSGTDFTLKISRVEAEDVGVYYC

SEQ ID NO: 89 - protein, 9 aa, Homo sapiens
MQGLQTPIT

SEQ ID NO: 90 - protein, 11 aa, Homo sapiens
FGQGTRLEIKR

SEQ ID NO: 91 - DNA, 366 bp, Homo sapiens
caggtgcagctggtggagtctgggggaggcgtggtccagcctgggaggtccctgagactctcctgtgcagcgtctggattcaccttcagtaactatggcattcactgggtccgccaggctccaggcaaggggctggagtgggtggcagttatatggtttgatggacgtaataaatactatgcagactccgtgaagggccgattcaccatctccagagacaattccaagaacacgctgtatctgcaaatgaacagcctgagagccgaggacgcggctgtgtattactgtgcgagagatccctttgactatggtgactccttctttgactactggggccagggcaccctggtcaccgtctcctcagcc

SEQ ID NO: 92 - protein, 122 aa, Homo sapiens
QVQLVESGGGVVQPGRSLRLSCAASGFTFSNYGIHWVRQAPGKGLEWVAVIWFDGRNKYYADSVKGRFTISRDNSKNTLYLQMNSLRAEDAAVYYCARDPFDYGDSFFDYWGQGTLVTVSSA

SEQ ID NO: 93 - protein, 25 aa, Homo sapiens
QVQLVESGGGVVQPGRSLRLSCAAS

SEQ ID NO: 94 - protein, 10 aa, Homo sapiens
GFTFSNYGIH

SEQ ID NO: 95 - protein, 14 aa, Homo sapiens
WVRQAPGKGLEWVA

SEQ ID NO: 96 - protein, 17 aa, Homo sapiens
VIWFDGRNKYYADSVKG

SEQ ID NO: 97 - protein, 32 aa, Homo sapiens
RFTISRDNSKNTLYLQMNSLRAEDAAVYYCAR

SEQ ID NO: 98 - protein, 12 aa, Homo sapiens
DPFDYGDSFFDY

SEQ ID NO: 99 - protein, 12 aa, Homo sapiens
WGQGTLVTVSSA

SEQ ID NO: 100 - DNA, 315 bp, Homo sapiens
ctgactcagtctccatcctccctgtctgcatctgtaagagacagagtcaccatcacttgccgggcgagtcaggacattagcaattatttagcctggtatcagcagaaaccagggaaagttcctaatctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgctcactttcggcggagggaccaaggtggagatcaaacga

SEQ ID NO: 101 - protein, 105 aa, Homo sapiens
LTQSPSSLSASVRDRVTITCRASQDISNYLAWYQQKPGKVPNLLIYAASTLQSGVPSRFSGSGSGTDFTLTISSLQPEDVATYYCQKYNSAPLTFGGGTKVEIKR

SEQ ID NO: 102 - protein, 20 aa, Homo sapiens
LTQSPSSLSASVRDRVTITC

SEQ ID NO: 103 - protein, 11 aa, Homo sapiens
RASQDISNYLA

SEQ ID NO: 104 - protein, 15 aa, Homo sapiens
WYQQKPGKVPNLLIY

SEQ ID NO: 105 - protein, 6 aa, Homo sapiens
AASTLQ

SEQ ID NO: 106 - protein, 32 aa, Homo sapiens
GVPSRFSGSGSGTDFTLTISSLQPEDVATYYC

SEQ ID NO: 107 - protein, 9 aa, Homo sapiens
QKYNSAPLT

SEQ ID NO: 108 - protein, 11 aa, Homo sapiens
FGGGTKVEIKR

SEQ ID NO: 109 - DNA, 366 bp, Homo sapiens
caggtgcagctggtggagtctgggggaggcttggtcaagcctggagggtccctgagactctcctgtgcagcctctggattcaccttcagtgactactacatgacctggatccgccaggctccagggaaggggctggagtgggtttcatacattagtattagtggtagtatcacacactacgcagactcagtgaagggccgattcaccatgtccagggacaacgccaagaactcactgtatctgcaaatgaacagcctgagagccgaggacacggccgtgtattactgtgcgagagacggagcagcagctggtacggatgcttttgatatctggggccacgggacaaaggtcaccgtctcttcagcc

SEQ ID NO: 110 - protein, 122 aa, Homo sapiens
QVQLVESGGGLVKPGGSLRLSCAASGFTFSDYYMTWIRQAPGKGLEWVSYISISGSITHYADSVKGRFTMSRDNAKNSLYLQMNSLRAEDTAVYYCARDGAAAGTDAFDIWGHGTKVTVSSA

SEQ ID NO: 111 - protein, 25 aa, Homo sapiens
QVQLVESGGGLVKPGGSLRLSCAAS

SEQ ID NO: 112 - protein, 10 aa, Homo sapiens
GFTFSDYYMT

SEQ ID NO: 113 - protein, 14 aa, Homo sapiens
WIRQAPGKGLEWVS

SEQ ID NO: 114 - protein, 17 aa, Homo sapiens
YISISGSITHYADSVKG

SEQ ID NO: 115 - protein, 32 aa, Homo sapiens
RFTMSRDNAKNSLYLQMNSLRAEDTAVYYCAR

SEQ ID NO: 116 - protein, 12 aa, Homo sapiens
DGAAAGTDAFDI

SEQ ID NO: 117 - protein, 12 aa, Homo sapiens
WGHGTKVTVSSA

SEQ ID NO: 118 - DNA, 321 bp, Homo sapiens
gagatagtgatgacgcagtctccagccaccctatctgtgtctccaggggacagagccaccctctcctgcagggccagtcagaatgttagcagcaacttggcctggtaccagcagaaacctggccaggctcccaggctcctcatctttggtgcatccaccagggccactggtatcccagccaggttcagtggcagtgggtctgggacagagttcactctcaccatcagcagcctacagtctgaagattttgcagtttattactgtcagcagtatcattactggcccactttcggccctgggaccaaagtggatatcaaacga

SEQ ID NO: 119 - protein, 107 aa, Homo sapiens
EIVMTQSPATLSVSPGDRATLSCRASQNVSSNLAWYQQKPGQAPRLLIFGASTRATGIPARFSGSGSGTEFTLTISSLQSEDFAVYYCQQYHYWPTFGPGTKVDIKR

SEQ ID NO: 120 - protein, 23 aa, Homo sapiens
EIVMTQSPATLSVSPGDRATLSC

SEQ ID NO: 121 - protein, 11 aa, Homo sapiens
RASQNVSSNLA

SEQ ID NO: 122 - protein, 15 aa, Homo sapiens
WYQQKPGQAPRLLIF

SEQ ID NO: 123 - protein, 7 aa, Homo sapiens
GASTRAT

SEQ ID NO: 124 - protein, 32 aa, Homo sapiens; identical to SEQ ID NO: 34

SEQ ID NO: 125 - protein, 8 aa, Homo sapiens
QQYHYWPT

SEQ ID NO: 126 - protein, 11 aa, Homo sapiens
FGPGTKVDIKR

SEQ ID NO: 127 - DNA, 360 bp, Homo sapiens
cagctggtgcagtctggggctgaggtgaagaagcctggggcctcagtgaaggtctcctgcaaggcttctggatacaccttcaccggcttctatatgcactgggtgcgacagacccctggacaagggcttgagtggatgggatggatcaaccctaacagtggtggcacatattatgtacagaagtttcagggcagggtcaccatgaccagggacacgtccatcagcacagtctacatggagctgagcaggttgagatctgacgacacggccgtatattactgtgcgagagatgggtatagcagtggagaggactggttcgacccctggggccagggaaccctggtcaccgtctcctcagcc

SEQ ID NO: 128 - protein, 120 aa, Homo sapiens
QLVQSGAEVKKPGASVKVSCKASGYTFTGFYMHWVRQTPGQGLEWMGWINPNSGGTYYVQKFQGRVTMTRDTSISTVYMELSRLRSDDTAVYYCARDGYSSGEDWFDPWGQGTLVTVSSA

SEQ ID NO: 129 - protein, 23 aa, Homo sapiens
QLVQSGAEVKKPGASVKVSCKAS

SEQ ID NO: 130 - protein, 10 aa, Homo sapiens
GYTFTGFYMH

SEQ ID NO: 131 - protein, 14 aa, Homo sapiens
WVRQTPGQGLEWMG

SEQ ID NO: 132 - protein, 17 aa, Homo sapiens
WINPNSGGTYYVQKFQG

SEQ ID NO: 133 - protein, 32 aa, Homo sapiens
RVTMTRDTSISTVYMELSRLRSDDTAVYYCAR

SEQ ID NO: 134 - protein, 12 aa, Homo sapiens
DGYSSGEDWFDP

SEQ ID NO: 135 - protein, 12 aa, Homo sapiens
WGQGTLVTVSSA

SEQ ID NO: 136 - DNA, 339 bp, Homo sapiens
gatattgtgatgacccagactccactctctctgtccgtcacccctggacagccggcctccatctcctgcaagtctagtcagagcctcctgcatagtggtggaaagacctatttgtattggtacctgcagaggccaggccagcctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacaccttccgctcactttcggcggagggaccaaggtggagatcaaacga

SEQ ID NO: 137 - protein, 113 aa, Homo sapiens
DIVMTQTPLSLSVTPGQPASISCKSSQSLLHSGGKTYLYWYLQRPGQPPQLLIYEVSNRFSGVPDRFSGSGSGTDFTLKISRVEAEDVGVYYCMQSIHLPLTFGGGTKVEIKR

SEQ ID NO: 138 - protein, 23 aa, Homo sapiens
DIVMTQTPLSLSVTPGQPASISC

SEQ ID NO: 139 - protein, 16 aa, Homo sapiens
KSSQSLLHSGGKTYLY

SEQ ID NO: 140 - protein, 15 aa, Homo sapiens
WYLQRPGQPPQLLIY

SEQ ID NO: 141 - protein, 7 aa, Homo sapiens
EVSNRFS

SEQ ID NO: 142 - protein, 32 aa, Homo sapiens; identical to SEQ ID NO: 88

SEQ ID NO: 143 - protein, 9 aa, Homo sapiens
MQSIHLPLT

SEQ ID NO: 144 - protein, 11 aa, Homo sapiens
FGGGTKVEIKR

SEQ ID NO: 145 - DNA, 360 bp, Homo sapiens
caggtgcagctggagcagtcggggggaggcctggtcaagcctggggggtccctgagattctcctgtgcagcctctggattcaccttcagtagctatagcatgaactgggtccgccaggctccagggaaggggctggagtgggtctcattcattagtagtagtagtagttacatatactacgcagactcagtgaagggccgattcaccatctccagagacaacgccaagaactcactgtatctgcaaatgaacagcctgagagccgaggacacggctgtgtattactgtgcgagagaggactgggtgggagctacctttgactactggggccagggaaccctggtcaccgtctcctcagcc

SEQ ID NO: 146 - protein, 120 aa, Homo sapiens
QVQLEQSGGGLVKPGGSLRFSCAASGFTFSSYSMNWVRQAPGKGLEWVSFISSSSSYIYYADSVKGRFTISRDNAKNSLYLQMNSLRAEDTAVYYCAREDWVGATFDYWGQGTLVTVSSA

SEQ ID NO: 147 - protein, 25 aa, Homo sapiens
QVQLEQSGGGLVKPGGSLRFSCAAS

SEQ ID NO: 148 - protein, 10 aa, Homo sapiens
GFTFSSYSMN

SEQ ID NO: 149 - protein, 14 aa, Homo sapiens
WVRQAPGKGLEWVS

SEQ ID NO: 150 - protein, 17 aa, Homo sapiens
FISSSSSYIYYADSVKG

SEQ ID NO: 151 - protein, 32 aa, Homo sapiens
RFTISRDNAKNSLYLQMNSLRAEDTAVYYCAR

SEQ ID NO: 152 - protein, 10 aa, Homo sapiens
EDWVGATFDY

SEQ ID NO: 153 - protein, 12 aa, Homo sapiens
WGQGTLVTVSSA

SEQ ID NO: 154 - DNA, 324 bp, Homo sapiens
gacattcagctgacccagtctccatcctccctgtctgcatctgtaggagacagagtcaccatcacttgtcgggcgagtcagggcattaggaattatttagcctggtatcagcagaaaccagggaaagttcctaagctcctgatctatgctgcttccgctttgaaattaggggtcccatctcggttcagtggcagtggatctgggacagatttcactctcaccatcagcagcctgcagcctgaagatgttgcaacttattactgtcaaaagtataacagtgccccgatcaccttcggccaagggacacgactggacattaaacga

SEQ ID NO: 155 - protein, 108 aa, Homo sapiens
DIQLTQSPSSLSASVGDRVTITCRASQGIRNYLAWYQQKPGKVPKLLIYAASALKLGVPSRFSGSGSGTDFTLTISSLQPEDVATYYCQKYNSAPITFGQGTRLDIKR

SEQ ID NO: 156 - protein, 23 aa, Homo sapiens
DIQLTQSPSSLSASVGDRVTITC

SEQ ID NO: 157 - protein, 11 aa, Homo sapiens
RASQGIRNYLA

SEQ ID NO: 158 - protein, 15 aa, Homo sapiens
WYQQKPGKVPKLLIY

SEQ ID NO: 159 - protein, 7 aa, Homo sapiens
AASALKL

SEQ ID NO: 160 - protein, 32 aa, Homo sapiens; identical to SEQ ID NO: 106

SEQ ID NO: 161 - protein, 9 aa, Homo sapiens
QKYNSAPIT

SEQ ID NO: 162 - protein, 11 aa, Homo sapiens
FGQGTRLDIKR

SEQ ID NO: 163 - DNA, 396 bp, Homo sapiens
caggtgcagctggagcagtcgggcccaggactggtgaagccttcacagaacctgtccctcacctgcactgtctctggtggctccatcagcagtggtggttatttctggagctggatccgccagcacccagggaagggcctggagtggattgggtacatctattacagtgggaacacctactacaacccgtccctcaagagtcgagttaccatatcagttgacacgtctaagaaccagttctccctgaaactgagctctgtgactgccgcggacacggccgtgtattactgtgcgagagactattactatgatactagtggtttttcctaccgttacgactggtactacggtatggacgtctggggccaagggaccacggtcaccgtctcctcagcc

SEQ ID NO: 164 - protein, 132 aa, Homo sapiens
QVQLEQSGPGLVKPSQNLSLTCTVSGGSISSGGYFWSWIRQHPGKGLEWIGYIYYSGNTYYNPSLKSRVTISVDTSKNQFSLKLSSVTAADTAVYYCARDYYYDTSGFSYRYDWYYGMDVWGQGTTVTVSSA

SEQ ID NO: 165 - protein, 25 aa, Homo sapiens
QVQLEQSGPGLVKPSQNLSLTCTVS

SEQ ID NO: 166 - protein, 12 aa, Homo sapiens
GGSISSGGYFWS

SEQ ID NO: 167 - protein, 14 aa, Homo sapiens
WIRQHPGKGLEWIG

SEQ ID NO: 168 - protein, 16 aa, Homo sapiens
YIYYSGNTYYNPSLKS

SEQ ID NO: 169 - protein, 32 aa, Homo sapiens; identical to SEQ ID NO: 7

SEQ ID NO: 170 - protein, 21 aa, Homo sapiens
DYYYDTSGFSYRYDWYYGMDV

SEQ ID NO: 171 - protein, 12 aa, Homo sapiens
WGQGTTVTVSSA

SEQ ID NO: 172 - DNA, 321 bp, Homo sapiens
gacatccagctgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaaatggggtcccatcaaggttcagcggcagtggatctgggacagaattcactctcacaatcagcagcctgcagcctgaagattttgcaacttattactgtctacaacataatacttacccggcgttcggccaagggaccaaggtggaaatcaaacga

SEQ ID NO: 173 - protein, 107 aa, Homo sapiens
DIQLTQSPSSLSASVGDRVTITCRASQGIRNDLGWYQQKPGKAPKRLIYAASSLQNGVPSRFSGSGSGTEFTLTISSLQPEDFATYYCLQHNTYPAFGQGTKVEIKR

SEQ ID NO: 174 - protein, 23 aa, Homo sapiens
DIQLTQSPSSLSASVGDRVTITC

SEQ ID NO: 175 - protein, 11 aa, Homo sapiens
RASQGIRNDLG

SEQ ID NO: 176 - protein, 15 aa, Homo sapiens
WYQQKPGKAPKRLIY

SEQ ID NO: 177 - protein, 7 aa, Homo sapiens
AASSLQN

SEQ ID NO: 178 - protein, 32 aa, Homo sapiens
GVPSRFSGSGSGTEFTLTISSLQPEDFATYYC

SEQ ID NO: 179 - protein, 8 aa, Homo sapiens
LQHNTYPA

SEQ ID NO: 180 - protein, 11 aa, Homo sapiens
FGQGTKVEIKR

SEQ ID NO: 181 - DNA, 354 bp, Homo sapiens
cagctggtgcagtctggagcagaagtgaaaaagcccggggagtctctgaagatctcctgtcagggttctggatacatctttaccaactactggatcggctgggtgcgccagatgcccgggaaaggcctggagtggatgggggtcatctatcctgatgactctgataccagatacagcccgtccttccaaggccaggtcaccatctcagccgacaagtccatcagcaccgcctacctgcagtggagcagcctgaaggcctcggacaccgccatatattactgtgcgagacaaaaatggctacaacacccctttgactactggggccagggaaccctggtcaccgtctcctcagcc

SEQ ID NO: 182 - protein, 118 aa, Homo sapiens
QLVQSGAEVKKPGESLKISCQGSGYIFTNYWIGWVRQMPGKGLEWMGVIYPDDSDTRYSPSFQGQVTISADKSISTAYLQWSSLKASDTAIYYCARQKWLQHPFDYWGQGTLVTVSSA

SEQ ID NO: 183 - protein, 23 aa, Homo sapiens
QLVQSGAEVKKPGESLKISCQGS

SEQ ID NO: 184 - protein, 10 aa, Homo sapiens
GYIFTNYWIG

SEQ ID NO: 185 - protein, 14 aa, Homo sapiens
WVRQMPGKGLEWMG

SEQ ID NO: 186 - protein, 17 aa, Homo sapiens
VIYPDDSDTRYSPSFQG

SEQ ID NO: 187 - protein, 32 aa, Homo sapiens
QVTISADKSISTAYLQWSSLKASDTAIYYCAR

SEQ ID NO: 188 - protein, 10 aa, Homo sapiens
QKWLQHPFDY

SEQ ID NO: 189 - protein, 12 aa, Homo sapiens
WGQGTLVTVSSA

SEQ ID NO: 190 - DNA, 327 bp, Homo sapiens
gaaattgtgttgacgcagtcaccaggcaccctgtctttgtctccaggggaaagagtcaccctctcatgcagggccagtcagagtgttagcagcagatacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtttattactgtcagcagtatggtagctcacctcggacgttcggccaagggaccaaggtggaaatcaaacga

SEQ ID NO: 191 - protein, 109 aa, Homo sapiens
EIVLTQSPGTLSLSPGERVTLSCRASQSVSSRYLAWYQQKPGQAPRLLIYGASSRATGIPDRFSGSGSGTDFTLTISRLEPEDFAVYYCQQYGSSPRTFGQGTKVEIKR

SEQ ID NO: 192 - protein, 23 aa, Homo sapiens
EIVLTQSPGTLSLSPGERVTLSC

SEQ ID NO: 193 - protein, 12 aa, Homo sapiens
RASQSVSSRYLA

SEQ ID NO: 194 - protein, 15 aa, Homo sapiens
WYQQKPGQAPRLLIY

SEQ ID NO: 195 - protein, 7 aa, Homo sapiens
GASSRAT

SEQ ID NO: 196 - protein, 31 aa, Homo sapiens
GIPDRFSGSGSGTDFTLTISRLEPEDFAVYY

SEQ ID NO: 197 - protein, 9 aa, Homo sapiens
QQYGSSPRT

SEQ ID NO: 198 - protein, 11 aa, Homo sapiens
FGQGTKVEIKR

SEQ ID NO: 199 - DNA, 369 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcaggacagggtcaccatgaccagggacacgtccatcagcacagcctacatggagctgagcaggctgagatctgacgacacggccgtgtattactgtgcgagagatttctttggttcggggagtctcctctactttgactactggggccagggaaccctggtcaccgtctcctcagcc

SEQ ID NO: 200 - protein, 123 aa, Homo sapiens
QVQLVQSGAEVKKPGASVKVSCKASGYTFTGYYMHWVRQAPGQGLEWMGWINPNSGGTNYAQKFQDRVTMTRDTSISTAYMELSRLRSDDTAVYYCARDFFGSGSLLYFDYWGQGTLVTVSSA

SEQ ID NO: 201 - protein, 25 aa, Homo sapiens [sequence omitted]

SEQ ID NO: 202 - protein, 10 aa, Homo sapiens
GYTFTGYYMH

SEQ ID NO: 203 - protein, 14 aa, Homo sapiens
WVRQAPGQGLEWMG

SEQ ID NO: 204 - protein, 17 aa, Homo sapiens
WINPNSGGTNYAQKFQD

SEQ ID NO: 205 - protein, 32 aa, Homo sapiens
RVTMTRDTSISTAYMELSRLRSDDTAVYYCAR

SEQ ID NO: 206 - protein, 13 aa, Homo sapiens
DFFGSGSLLYFDY

SEQ ID NO: 207 - protein, 12 aa, Homo sapiens
WGQGTLVTVSSA

SEQ ID NO: 208 - DNA, 339 bp, Homo sapiens; identical to SEQ ID NO: 136

SEQ ID NO: 209 - protein, 113 aa, Homo sapiens; identical to SEQ ID NO: 137

SEQ ID NO: 210 - protein, 23 aa, Homo sapiens
DIVMTQTPLSLSVTPGQPASISC

SEQ ID NO: 211 - protein, 16 aa, Homo sapiens
KSSQSLLHSGGKTYLY

SEQ ID NO: 212 - protein, 15 aa, Homo sapiens
WYLQRPGQPPQLLIY

SEQ ID NO: 213 - protein, 7 aa, Homo sapiens
EVSNRFS

SEQ ID NO: 214 - protein, 32 aa, Homo sapiens; identical to SEQ ID NO: 88

SEQ ID NO: 215 - protein, 9 aa, Homo sapiens
MQSIHLPLT

SEQ ID NO: 216 - protein, 11 aa, Homo sapiens
FGGGTKVEIKR

SEQ ID NO: 217 - DNA, 366 bp, Homo sapiens
caggtgcagctggagcagtcggggggaggcgtggtccagcctgggaggtccctgagactctcctgtgcagcgtctggattcaccttcaataactatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgaaagatgaggaatactactatgtttcggggcttgactactggggccagggaaccctggtcaccgtctcctcagcc

SEQ ID NO: 218 - protein, 122 aa, Homo sapiens
QVQLEQSGGGVVQPGRSLRLSCAASGFTFNNYGMHWVRQAPGKGLEWVAVIWYDGSNKYYADSVKGRFTISRDNSKNTLYLQMNSLRAEDTAVYYCAKDEEYYYVSGLDYWGQGTLVTVSSA

SEQ ID NO: 219 - protein, 25 aa, Homo sapiens
QVQLEQSGGGVVQPGRSLRLSCAAS

SEQ ID NO: 220 - protein, 10 aa, Homo sapiens
GFTFNNYGMH

SEQ ID NO: 221 - protein, 14 aa, Homo sapiens
WVRQAPGKGLEWVA

SEQ ID NO: 222 - protein, 17 aa, Homo sapiens
VIWYDGSNKYYADSVKG

SEQ ID NO: 223 - protein, 32 aa, Homo sapiens
RFTISRDNSKNTLYLQMNSLRAEDTAVYYCAK

SEQ ID NO: 224 - protein, 12 aa, Homo sapiens
DEEYYYVSGLDY

SEQ ID NO: 225 - protein, 12 aa, Homo sapiens
WGQGTLVTVSSA

SEQ ID NO: 226 - DNA, 315 bp, Homo sapiens; identical to SEQ ID NO: 100

SEQ ID NO: 227 - protein, 105 aa, Homo sapiens; identical to SEQ ID NO: 101

SEQ ID NO: 228 - protein, 20 aa, Homo sapiens
LTQSPSSLSASVRDRVTITC

SEQ ID NO: 229 - protein, 11 aa, Homo sapiens
RASQDISNYLA

SEQ ID NO: 230 - protein, 15 aa, Homo sapiens
WYQQKPGKVPNLLIY

SEQ ID NO: 231 - protein, 6 aa, Homo sapiens
AASTLQ

SEQ ID NO: 232 - protein, 32 aa, Homo sapiens; identical to SEQ ID NO: 106

SEQ ID NO: 233 - protein, 9 aa, Homo sapiens
QKYNSAPLT

SEQ ID NO: 234 - protein, 11 aa, Homo sapiens
FGGGTKVEIKR

SEQ ID NO: 235 - DNA, 372 bp, Homo sapiens
cagatcaccttgaaggagtctggtcctacgctggtgacacccacacagaccctcacgctgacctgcaccttctctgggttctcactcagcactggtggaatgggtgtgggctggatccgtcagcccccaggaaaggccctggactggcttacactcatttattggaatgatgataagcactacagcccatctctgaagagcaggcttaccatcaccaaggacacctccaaaaaccaggtggtccttagaatgaccaacatggaccctgtggacacagccacttattactgtgcacacctgcattacgatattttgactggttttaactttgactactggggccagggaaccctggtcaccgtctcctcagcc

SEQ ID NO: 236 - protein, 124 aa, Homo sapiens
QITLKESGPTLVTPTQTLTLTCTFSGFSLSTGGMGVGWIRQPPGKALDWLTLIYWNDDKHYSPSLKSRLTITKDTSKNQVVLRMTNMDPVDTATYYCAHLHYDILTGFNFDYWGQGTLVTVSSA

SEQ ID NO: 237 - protein, 25 aa, Homo sapiens
QITLKESGPTLVTPTQTLTLTCTFS

SEQ ID NO: 238 - protein, 12 aa, Homo sapiens
GFSLSTGGMGVG

SEQ ID NO: 239 - protein, 14 aa, Homo sapiens
WIRQPPGKALDWLT

SEQ ID NO: 240 - protein, 16 aa, Homo sapiens
LIYWNDDKHYSPSLKS

SEQ ID NO: 241 - protein, 32 aa, Homo sapiens
RLTITKDTSKNQVVLRMTNMDPVDTATYYCAH

SEQ ID NO: 242 - protein, 13 aa, Homo sapiens
LHYDILTGFNFDY

SEQ ID NO: 243 - protein, 12 aa, Homo sapiens
WGQGTLVTVSSA

SEQ ID NO: 244 - DNA, 342 bp, Homo sapiens
gatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgatttcacactgaaaatcagcagggtggaggctgaggatgttggagtttattactgcatgcaacgtatagagtttccgctcactttcggcggagggaccaaggtggagatcaaacga

SEQ ID NO: 245 - protein, 114 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKR

SEQ ID NO: 246 - protein, 23 aa, Homo sapiens
DIVMTQTPLSLPVTPGEPASISC

SEQ ID NO: 247 - protein, 17 aa, Homo sapiens
RSSQSLLDSDDGNTYLD

SEQ ID NO: 248 - protein, 15 aa, Homo sapiens
WYLQKPGQSPQLLIY

SEQ ID NO: 249 - protein, 7 aa, Homo sapiens
TLSYRAS

SEQ ID NO: 250 - protein, 32 aa, Homo sapiens; identical to SEQ ID NO: 88

SEQ ID NO: 251 - protein, 9 aa, Homo sapiens
MQRIEFPLT

SEQ ID NO: 252 - protein, 11 aa, Homo sapiens
FGGGTKVEIKR

SEQ ID NO: 253 - DNA, 369 bp, Homo sapiens; identical to SEQ ID NO: 199

SEQ ID NO: 254 - protein, 123 aa, Homo sapiens; identical to SEQ ID NO: 200

SEQ ID NO: 255 - protein, 25 aa, Homo sapiens [sequence omitted]

SEQ ID NO: 256 - protein, 10 aa, Homo sapiens
GYTFTGYYMH

SEQ ID NO: 257 - protein, 14 aa, Homo sapiens
WVRQAPGQGLEWMG

SEQ ID NO: 258 - protein, 17 aa, Homo sapiens
WINPNSGGTNYAQKFQD

SEQ ID NO: 259 - protein, 32 aa, Homo sapiens; identical to SEQ ID NO: 205

SEQ ID NO: 260 - protein, 13 aa, Homo sapiens
DFFGSGSLLYFDY

SEQ ID NO: 261 - protein, 12 aa, Homo sapiens
WGQGTLVTVSSA

SEQ ID NO: 262 - DNA, 339 bp, Homo sapiens; identical to SEQ ID NO: 136

SEQ ID NO: 263 - protein, 113 aa, Homo sapiens; identical to SEQ ID NO: 137

SEQ ID NO: 264 - protein, 23 aa, Homo sapiens
DIVMTQTPLSLSVTPGQPASISC

SEQ ID NO: 265 - protein, 16 aa, Homo sapiens
KSSQSLLHSGGKTYLY

SEQ ID NO: 266 - protein, 15 aa, Homo sapiens
WYLQRPGQPPQLLIY

SEQ ID NO: 267 - protein, 7 aa, Homo sapiens
EVSNRFS

SEQ ID NO: 268 - protein, 32 aa, Homo sapiens; identical to SEQ ID NO: 88

SEQ ID NO: 269 - protein, 9 aa, Homo sapiens
MQSIHLPLT

SEQ ID NO: 270 - protein, 11 aa, Homo sapiens
FGGGTKVEIKR

SEQ ID NO: 271 - protein, 470 aa, Homo sapiens
KRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPAS

SEQ ID NO: 272 - protein, 445 aa, Homo sapiens
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK